SEQUENCE LISTING (5 sequences)

SEQ ID NO: 1 - protein, 236 aa, Artificial
  Synthetic
DKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 232 aa, Artificial
  Synthetic
DKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 233 aa, Artificial
  Synthetic
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 235 aa, Artificial
  Synthetic
DKKAEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 232 aa, Artificial
  Synthetic
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2015/0203591 A1
(43) Pub. Date: Jul. 23, 2015

(54) MUTIVALENT ANTIGEN-BINDING PROTEINS

(71) Applicant: REGENERON PHARMACEUTICALS, INC., Tarrytown, NY (US)

(72) Inventors: George D. Yancopoulos, Yorktown Heights, NY (US); Nicholas J. Papadopoulos, LaGrangeville, NY (US); Neil Stahl, Carmel, NY (US); Samuel Davis, New York, NY (US); Andrew J. Murphy, Croton-on-Hudson, NY (US); Lynn Macdonald, White Plains, NY (US)

(73) Assignee: REGENERON PHARMACEUTICALS, INC., Tarrytown, NY (US)

(21) Appl. No.: 14/417,863

(22) PCT Filed: Jul. 31, 2013
(86) PCT No.: PCT/US13/52985

§ 371 Date: Jan. 28, 2015

Related U.S. Application Data
(60) Provisional application No. 61/759,578, filed on Feb. 1, 2013.
(60) Provisional application No. 61/736,810, filed on Dec. 13, 2012.
(60) Provisional application No. 61/678,944, filed on Aug. 2, 2012.

(51) Int. Cl. C07K 16/46; C07K 16/28

(52) U.S. Cl. CPC C07K 16/468; C07K 16/2887; C07K 16/2809; C07K 2317/31; C07K 2317/522; C07K 2317/21; C07K 2317/524; C07K 2317/526; C07K 2317/24; C07K 2317/622; C07K 2317/64

(57) ABSTRACT

Multivalent antigen-binding proteins comprising two or three or four or more immunoglobulin heavy chain variable domain binding domains are provided, as are methods for making them, nucleic acid constructs, and cell lines for making them. Proteins comprising two or three or four or more different heavy chain variable domains that lack an immunoglobulin variable domain are provided. Proteins comprising two or three or four or more different heavy chain variable domains that associate with the same immunoglobulin light chain variable domain are also provided.

### CROSS REFERENCE TO RELATED APPLICATIONS

This application claims the benefit of priority to U.S. Provisional Application No. 61/678,944, filed 2 Aug. 2012, U.S. Provisional Application No. 61/736,810, filed 13 Dec. 2012, and U.S. Provisional Application No. 61/759,578, filed 1 Feb. 2013. Each of these applications is incorporated by reference herein in its entirety.

### FIELD OF INVENTION

Antigen-binding proteins, including proteins that comprise immunoglobulin heavy and/or light chain variable domains that bind an antigen of interest. Multivalent antigen-binding proteins, such as bispecific and trispecific antigen-binding proteins, comprise two or more antigen-binding domains that bind one, two or more epitopes of the same or different antigen. Methods for making antigen-binding proteins having two or three or four antigen-binding domains, wherein the two or three or four antigen-binding domains comprise immunoglobulin heavy chain variable domains alone or in combination with an immunoglobulin heavy chain variable domain. Multivalent antigen-binding proteins comprising specialized immunoglobulin binding domains made in humanized non-human animals.

### BACKGROUND

Antigen-binding proteins that are based on immunoglobulin sequences, e.g., multivalent antibodies and other immunoglobulin-based binding proteins (see, e.g., U.S. Pat. No. 8,298,532) are known in the art. Multivalent antigen-binding proteins are useful for making molecules, e.g., therapeutic molecules, which exhibit desired functionalities. There is a need in the art for new and useful formats for multivalent antigen-binding proteins.

### SUMMARY

Multivalent antigen-binding proteins that comprise immunoglobulin-based binding moieties are provided. Antigen-binding proteins comprising two or three or four immunoglobulin heavy chain and/or light chain variable domain binding regions are provided, as are methods for making them, nucleic acid constructs, and cell lines for making them. Non-human animals with selectively engineered immunoglobulin loci are provided, such as non-human animals with humanized immunoglobulin variable region sequences that generate human variable domains that are suitable for novel multivalent antigen-binding molecule formats.

Multivalent antigen-binding protein comprising two, three, or four or more heavy chain immunoglobulin variable domains are provided, wherein the two, three, or four or more heavy chain immunoglobulin variable domains are each paired with a light chain variable domain derived from the same light chain V gene segment, e.g., a common or universal light chain variable domain. In various embodiments, the heavy chain variable domains are derived from mice that are genetically modified to express an immunoglobulin light chain repertoire derived from no more than one, or no more than two, immunoglobulin light chain V gene segments. In one embodiment, the light chain V gene segment is a human Vκ gene segment. In one embodiment, the light chain V gene segment is a human Vλ gene segment.

Multivalent antigen-binding proteins are provided that comprise two, three, or four or more immunoglobulin heavy chain variable domains (e.g., single variable domains), wherein the proteins lack a light chain. In various embodiments, the proteins further lack a C_{H}1 domain. In various embodiments, the heavy chain variable domains are derived from mice that lack a nucleic acid sequence that encodes an IgG1 C_{H}1 domain, and, in specific embodiments, that comprise a nucleic acid sequence that encodes an IgM C_{H}1 domain. Suitable heavy chain variable domains are heavy chain variable domains that capable of binding an antigen of interest in the absence of a cognate light chain variable domain. In various embodiments, the heavy chain variable domains are operably linked to a constant region lacking a C_{H}1 domain. In various embodiments, the constant region comprises a hinge, a C_{H}2, a C_{H}3 or a combination thereof.

Multivalent antigen-binding proteins are provided that comprise two, three, or four or more immunoglobulin light chain variable domains (e.g., single variable domains), wherein the light chain variable domains lack a cognate heavy chain variable domain. Such variable domains are capable of specifically binding an antigen (or an epitope on an antigen) in the absence of a heavy chain variable domain. In various embodiments, the light chain variable domains are derived from the same light chain V gene segment. In one embodiment, the light chain V gene segment is a human Vκ gene segment.

Multivalent antigen-binding proteins that comprise combinations of the above, e.g., the contain heavy chain immunoglobulin single variable domains, light chain immunoglobulin single variable domains, and cognate heavy chain/light chain variable domains, are also provided.

In various aspects, the novel formats and combinations are made possible using genetically engineered non-human animals with modified immunoglobulin variable region loci, such as humanized rodents, e.g., VELOCIMMUNE® mice, and mice that comprise restricted heavy and/or light chain variable loci.

Nucleic acids that encode such proteins, and cells and tissues that express them, are also provided.

Methods for making and using multivalent antigen-binding proteins are also provided.

### BRIEF DESCRIPTION OF THE FIGURES

FIG. 1A illustrates an embodiment of a multivalent antigen-binding protein comprising four immunoglobulin heavy chain variable domains (V_{H}1, V_{H}2, V_{H}3, and V_{H}4), a C_{H}1, a hinge or linker, and multimerizing components M1 and M2, associated with two copies of a polypeptide comprising two tandem copies of an immunoglobulin light chain variable domain (V_{L}, optionally separated by a linker) and an immunoglobulin light chain constant domain (C_{L}).

FIG. 1B illustrates an embodiment of a multivalent antigen-binding protein comprising four immunoglobulin heavy chain variable domains (V_{H}1, V_{H}2, V_{H}3, and V_{H}4), a C_{H}1 and/or a hinge and/or a linker, and multimerizing components M1 and M2, associated with two copies of a polypeptide comprising an immunoglobulin light chain constant sequence (C_{L}) and a two distinct light chain variable domains connected directly or by a linker (V_{L}1 and V_{L}2).

FIG. 2A illustrates an embodiment of a multivalent antigen-binding protein as in FIG. 1A, but comprising instead only three immunoglobulin heavy chain variable domains (V_{H}1, V_{H}2, and V_{H}3), i.e., lacking a V_{H}4 domain.

FIG. 2B illustrates an embodiment of a multivalent antigen-binding protein similar to that shown in FIG. 1B, but lacking a second V_{L} on the light chain component. In one embodiment, V_{H}1 and V_{H}3 are single domain (sd) immunoglobulin heavy chain domains, e.g., domains derived from immunized non-human animals that lack a C_{H}1 or a C_{H}1 and hinge sequence of an Ig heavy chain.

FIG. 2C illustrates an embodiment of a multivalent antigen-binding protein that is similar to the protein of FIG. 1B, but that lacks a V_{H}1 and a V_{H}3 domain. In the embodiment shown the V_{L}1 is a single domain (sd) light chain variable domain.

FIG. 2D illustrates an embodiment of a multivalent antigen-binding protein that is similar to the protein of FIG. 1B, but that lacks and arm on M2, which is replaced by a heavy chain variable domain that is a single domain (sd) moiety (V_{H}3sd). A V_{H}3sd is made, e.g., in a non-human animal that lacks a C_{H}1 or a C_{H}1 and hinge sequence of an Ig heavy chain.

FIG. 3A illustrates an embodiment of a multivalent antigen-binding protein as in FIG. 1A, but lacking light chain variable and constant domains. Each of V_{H}1, V_{H}2, V_{H}3, and V_{H4} may be single domains, e.g., made in non-human animals which lack a C_{H}1 or a C_{H}1 and hinge sequence of an Ig heavy chain (e.g., single domains, or sd).

FIG. 3B illustrates an embodiment of a multivalent antigen-binding protein as in FIG. 3A, but wherein the binding domains are replaced with immunoglobulin light chain binding domains V_{L}1, V_{L}2, V_{L}3, and V_{L}4. Single V_{L} domains may be made in non-human animals that have a constrained immunoglobulin heavy chain repertoire, whose antibodies can be screened for light chains that specifically bind antigen. In various embodiments, they are referred to as single domain V_{L}s, or V_{L}sd's).

FIG. 4A illustrates an embodiment of a multivalent antigen-binding protein as in FIG. 3A, comprising only three V_{H}sd domains,

FIG. 4B illustrates an embodiment similar to the embodiment of FIG. 4A, but wherein the binding domains are immunoglobulin light chain single binding (sd) domains, such as those employed in FIG. 3B.

FIG. 5 illustrates an embodiment of a multivalent antigen-binding protein comprising a polypeptide having two scFv moieties in tandem (optionally connected by a linker) fused to a first multimerizing component (M1) and a second polypeptide comprising a single scFv moiety fused to a second multimerizing component (M2).

FIG. 6 illustrates a multivalent antigen-binding protein having a first polypeptide that comprises a first binding moiety (a V_{H}1-C_{H}1/V_{L}-C_{L} moiety) fused to one end of a first multimerizing component (M1), which is fused to a second binding moiety (a V_{H}2-C_{H}1/V_{L}-C_{L} moiety); and a second polypeptide comprising a third binding moiety (a V_{H}3-C_{H}1/V_{L}-C_{L} moiety) fused to one end of a second multimerizing component (M2), which is fused to a fourth binding moiety (a V_{H}4-C_{H}1/V_{L}-C_{L} moiety).

FIG. 7A illustrates a multivalent antigen-binding protein as in FIG. 6, but lacking a fourth binding moiety.

FIG. 7B illustrates a multivalent antigen-binding protein as in FIG. 7A, but wherein one of the binding arms of M1 is replaced with a single domain heavy chain immunoglobulin domain (V_{H}2sd).

FIG. 7C illustrates a multivalent antigen-binding protein as in FIG. 7B, but the single domain heavy chain immunoglobulin domain has been replaced with a single domain V_{L} binding domain (V_{L}2sd).

FIG. 7D illustrates a multivalent antigen-binding protein as in FIG. 7C, but each bottom arm of M1 and M2 comprise a single domain V_{L} binding domain (V_{L}2sd and V_{L}3sd).

FIG. 8A illustrates a multivalent antigen-binding protein comprising a first multimerizing component (M1) that has a first immunoglobulin heavy chain variable domain (V_{H}1sd) fused to one end and a second immunoglobulin heavy chain variable domain (V_{H}2sd) fused to the other end; and a second multimerizing component that has a third immunoglobulin heavy chain variable domain (V_{H}3sd) fused to one end and a fourth immunoglobulin heavy chain variable domain (V_{H}4sd) fused to the other end.

FIG. 8B illustrates a multivalent antigen-binding protein similar to the protein of FIG. 8A, but wherein each of the binding moieties comprises an immunoglobulin light chain variable domain (single domain, sd).

FIG. 9A illustrates a multivalent antigen-binding protein as in FIG. 8A, but lacking a fourth immunoglobulin heavy chain variable domain (V_{H}4sd).

FIG. 9B illustrates a multivalent antigen-binding protein as in FIG. 9A, but having immunoglobulin single light chain variable domains (V_{L}sd1, V_{L}sd2, V_{L}sd3) as binding entities instead of heavy chain variable domains.

FIG. 10 illustrates a multivalent antigen-binding protein comprising a first polypeptide having a first multimerizing component (M1) that has fused on one end a first scFv (scFv1) and on the other end a second scFv (scFv2); and a second polypeptide having a second multimerizing component (M2) that has fused to it a third scFv (scFv3).

FIG. 11 illustrates a comparison of IgG1 (SEQ ID NO: 1), IgG2 (SEQ ID NO: 2), and IgG4 (SEQ ID NO: 3) lower hinge sequences modified as indicated in bold font. A modified IgG4 sequence is set forth in SEQ ID NO: 5.

FIG. 12 illustrates a comparison of IgG1 (SEQ ID NO: 1), IgG2 (SEQ ID NO: 2), and IgG4 (SEQ ID NO: 3) lower hinge region modified as shown in bold to reduce an effector function. A modified IgG1 sequence is set forth in SEQ ID NO: 4.

FIG. 13 illustrates a multivalent antigen-binding protein that independently binds antigens AG1 and AG2 (Panel C), which is made from variable domain sequences generated in a first humanized universal light chain (ULC) mouse by immunizing the ULC mouse with a first antigen (AG1) to make an antigen-binding protein that binds AG1 through the heavy chain only (Panel A); and from variable domain sequences generated in a second humanized mouse that has human κ variable segments at an endogenous mouse heavy chain locus and a ULC (a “κ-onto-heavy×ULC” mouse), and immunizing the κ-onto-heavy×ULC mouse with a second antigen (AG2) to make an antigen-binding protein that binds AG2 through the Vκ only. Variable sequences encoding AG1- and AG2-binding domains are derived from the mice and employed to make the multivalent antigen-binding protein of Panel C, which binds AG1 with up to two valencies through the V_{H} only; and which binds AG2 with up to two valencies through Vκ only.

FIG. 14 illustrates a multivalent antigen-binding protein having a first polypeptide that comprises a first binding moiety (a V_{H}1-C_{H}1/V_{L}-C_{L} moiety) fused to one end of a first multimerizing component (M1), which is fused to a second binding moiety (a C_{H}1-V_{H}2 moiety); and a second polypeptide comprising a third binding moiety (a V_{H}3-C_{H}1/V_{L}-C_{L} moiety) fused to one end of a second multimerizing component (M2), which is fused to a fourth binding moiety (a C_{H}1-V_{H}4/C_{L}-V_{L}-moiety).

FIG. 15 illustrates a multivalent antigen-binding protein as in FIG. 14, but lacking a fourth binding moiety.

### DETAILED DESCRIPTION

The inventions described herein are not limited to particular methods, and experimental conditions described, which may vary. The terminology used herein is for the purpose of describing particular embodiments only, and is not intended to be limiting; the scope of the invention is addressed by the claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, particular methods and materials are now described. All publications and patent applications mentioned in this disclosure are hereby incorporated by reference.

Antigen-binding proteins comprising three or more immunoglobulin heavy chain single variable domain binding regions are provided, as are methods for making them, nucleic acid constructs, and cell lines for making them.

Antigen-binding proteins comprising one, two, or three or more immunoglobulin light chain single variable domains are provided, as are methods for making them, nucleic acid constructs, and cells lines for making them.

Antigen-binding proteins comprising novel formats and combinations of cognate heavy and light chain variable domains are provided, as are methods for making them, nucleic acid constructs, and cell lines for making them.

Antigen-binding proteins in various embodiments with various combinations of one or more of immunoglobulin heavy chain single variable domains, immunoglobulin light chain single variable domains, and cognate heavy and light chain variable domains are also provided, as well as methods for making them, nucleic acid constructs, and cell lines for making them.

The antigen-binding proteins are generally multivalent, indicating that they comprise two or more binding moieties. The two or more binding moieties may exhibit different specificities. Thus, embodiments of multivalent antigen-binding proteins include multispecific antigen-binding proteins.

The nucleic acid sequences employed to make the described variable domains may be placed in any suitable expression vector and, in appropriate circumstances, two or more vectors in a single host cell. Generally, structural genes encoding variable domains are cloned with appropriate linkers and/or immunoglobulin sequences and/or multimerizing components, and the genes are placed in operable linkage with a promoter in a suitable expression construct in a suitable cell line for expression.

The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g., a retinal cell that expresses a viral gene (e.g., a PER.C6™ cell).

The phrase “multimerizing component” includes a moiety that is capable of promoting association of two polypeptides, e.g., an Fc of an immunoglobulin, e.g., an Fc of a human immunoglobulin or a multimerizing fragment thereof. Where the multimerizing component is an Fc, the Fc can comprise modifications in immunoglobulin domains, including where the modifications affect one or more effector function of the binding protein (e.g., modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, in various embodiments the multimerizing component is an Fc and the antigen-binding protein exhibits enhanced serum half-life (as compared with the same Fc-containing protein without the recited modification(s)) and has a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 259I (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an antigen (e.g., recognizing the antigen with a K_{D} in the micromolar, nanomolar, or picomolar range), that is capable of expressing and secreting from a cell, and that comprises at least one CDR. A heavy chain immunoglobulin single variable domain includes a heavy chain domain that expresses and functions in the absence of a cognate light chain variable domain. In various embodiments, a heavy chain immunoglobulin single variable domain that specifically binds an antigen or epitope of interest can be made in a genetically modified non-human animal that lacks a C_{H}1 or lacks a C_{H}1 and hinge sequence in an IgG gene, wherein the non-human animal comprises unrearranged human V, D, and J segments that are capable of rearranging and forming a rearranged human heavy chain gene (e.g., a rearranged human V/D/J gene). Alternatively, a heavy chain immunoglobulin single variable domain can be made in a mouse that is incapable of making a λ or a κ immunoglobulin light chain.

The phrase “light chain” includes an immunoglobulin light chain variable domain, or V_{L} (or functional fragment thereof); and an immunoglobulin constant domain, or C_{L} (or functional fragment thereof) sequence from any organism. Unless otherwise specified may include a light chain selected from a human kappa, lambda, and a combination thereof. Light chain variable (V_{L}) domains typically include three light chain CDRs and four framework (FR) regions, unless otherwise specified. Generally, a full-length light chain includes, from amino terminus to carboxyl terminus, a V_{L} domain that includes FR1-CDR1-FR2-CDR2-FR3-CDR3-FR4, and a light chain constant domain. Light chains that can be used with this invention include those, e.g., that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

In various embodiments a suitable light chain is a universal light chain, or common light chain. In various embodiments, the universal light chain is a κ light chain selected from a Vκ1-39 and a Vκ3-20 light chain. In various embodiments, the universal light chain is a λ light chain selected from a Vλ1-40 and a Vλ2-14. In certain embodiments, the universal light chain comprises a human germline variable sequence or a human sequence that comprises one, two, three, four, or five or more somatic hypermutations. Suitable universal light chains, and methods for making them, are disclosed in, e.g., US Patent Application Publication Nos. 2012/0192300, 2012/021409, 2011/0195454, and U.S. Ser. No. 13/488,628 filed 5 Jun. 2012, each hereby incorporated by reference.

In various embodiments, an immunoglobulin light chain single variable domain can be made in a non-human animal that comprises a severely restricted repertoire of heavy chain genes, e.g., no more than one, or no more than two, rearranged heavy chain genes or heavy chain V gene segments. When such a non-human animal is exposed to an immunogen, the non-human animal mounts an immune response characterized by a plurality of different light chain rearrangements and an extremely limited heavy chain repertoire (in one embodiment, a heavy chain derived from a single heavy chain V segment). Immunoglobulins from such an immunized mice, or antigen-positive B cells, are identified and analyzed for light chain variable domains that specifically bind the antigen of interest in the absence of heavy chain. Such light chain single variable domains are useful in various embodiments herein, because they do not require a cognate heavy chain to specifically bind an antigen of interest.

Immunoglobulin light chain single variable domains can be used in the same multivalent antigen-binding proteins as heavy chain single variable domains and/or cognate pairs of (traditional) heavy and light chain variable domains.

The phrase “somatically mutated” includes reference to a nucleic acid sequence from a B cell that has undergone class-switching, wherein the nucleic acid sequence of an immunoglobulin variable region (e.g., a heavy chain variable domain or including a heavy chain CDR or FR sequence) in the class-switched B cell is not identical to the nucleic acid sequence in the B cell prior to class-switching, such as, for example, a difference in a CDR or framework nucleic acid sequence between a B cell that has not undergone class-switching and a B cell that has undergone class-switching. “Somatically mutated” includes reference to nucleic acid sequences from affinity-matured B cells that are not identical to corresponding sequences in B cells that are not affinity-matured (i.e., sequences in the genome of germline cells). The phrase “somatically mutated” also includes reference to a nucleic acid sequence from a B cell after exposure of the B cell to an antigen of interest, wherein the nucleic acid sequence differs from the corresponding nucleic acid sequence prior to exposure of the B cell to the antigen of interest. The phrase “somatically mutated” refers to sequences from antibodies that have been generated in an animal, e.g., a mouse having human immunoglobulin variable region nucleic acid sequences, in response to an antigen challenge, and that result from the selection processes inherently operative in such an animal.

### Multivalent Binding Proteins

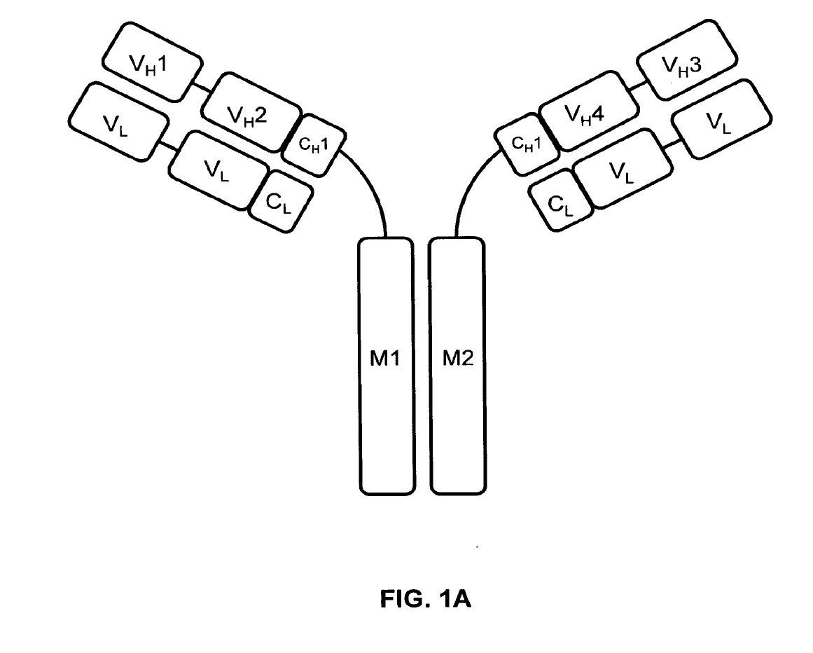

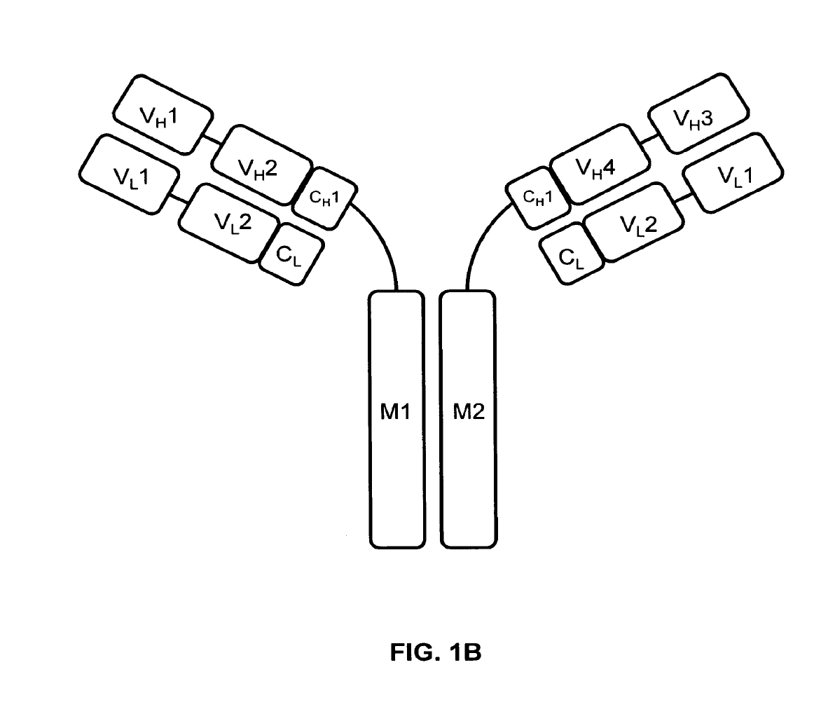

In one aspect, a multivalent antigen-binding protein is provided, comprising a first polypeptide that comprises a first immunoglobulin heavy chain variable domain (V_{H}1), a second heavy chain immunoglobulin variable domain (V_{H}2), a heavy chain C_{H}1 constant domain (C_{H}1), and a first multimerizing component (M1) (i.e., V_{H}1-V_{H}2-C_{H}1-M1); a second polypeptide that comprises a third immunoglobulin heavy chain variable domain (V_{H}3), a fourth immunoglobulin heavy chain variable domain (V_{H}4), a heavy chain C_{H}1 constant domain (C_{H}1), and a second multimerizing component (M2) (i.e., V_{H}3-V_{H}4-C_{H}1-M2); a third polypeptide comprising an immunoglobulin light chain variable domain (V_{L}) present in two copies (in one embodiment separated by a linker sequence), and an immunoglobulin light chain constant domain (C_{L}) V_{L}-V_{L}-C_{L}); wherein the first polypeptide associates with the second polypeptide by a multimerizing component, and wherein one third polypeptide molecule associates with the first polypeptide, and wherein one third polypeptide molecule associates with the second polypeptide (see, e.g., FIG. 1A).

In one embodiment, the multivalent antigen-binding protein consists essentially of three polypeptides. The first polypeptide consists essentially of a first heavy chain variable domain (V_{H}1) fused directly to through a linker to a second heavy chain variable domain that is fused directly or through a linker to a C_{H}1 region, which is fused directly or through a linker to a first multimerizing component. The second polypeptide consists essentially of a third heavy chain variable domain (V_{H}3) fused directly or through a linker to a fourth heavy chain variable domain (V_{H}4) that is fused directly or through a linker to a C_{H}1 region that is fused directly or through a linker to a second multimerizing component. The third polypeptide comprises a light chain variable domain (V_{L}) fused directly or through a linker to another light chain variable domain (V_{L}) that is fused directly or through a linker to a light chain constant region. In one embodiment, the first and the second light chain variable domains are the same. In one embodiment each light chain variable domain is cognate to each of V_{H}1, V_{H}2, V_{H}3, and V_{H}4.

In one embodiment, V_{H}1, V_{H}2, V_{H}3, and V_{H}4 are derived from a mouse that comprises a single rearranged light chain variable gene in its germline, such that each B cell of the mouse expresses a light chain derived from the same rearranged light chain gene; in such an embodiment, the light chain variable domain (V_{L}) will be cognate to each of V_{H}1, V_{H}2, V_{H}3, and V_{H}4 (made in the same mouse). In a specific embodiment, the light chain variable domain is derived from a Vκ1-39 gene segment or a Vλ3-20 gene segment, and the C_{L} is a Cκ. In a specific embodiment, the light chain variable domain is derived from a Vλ1-40 gene segment or a Vλ2-14 gene segment, and the C_{L} is a Cλ.

In one aspect, a multivalent antigen-binding protein is provided, comprising a first polypeptide that comprises a first immunoglobulin heavy chain variable domain (V_{H}1), a second heavy chain immunoglobulin variable domain (V_{H}2), a heavy chain C_{H}1 constant domain (C_{H}1), and a first multimerizing component (M1) V_{H}1-V_{H}2-C_{H}1-M1); a second polypeptide that comprises a third immunoglobulin heavy chain variable domain (VH3), a fourth immunoglobulin heavy chain variable domain (V_{H}4), a heavy chain C_{H}1 constant domain (C_{H}1), and a second multimerizing component (M2) (i.e., V_{H}3-V_{H}4-C_{H}1-M2); a third polypeptide comprising an immunoglobulin light chain variable domain (V_{L}1) linked directly or through a linker to a second immunoglobulin light chain variable domain (V_{L}2) that is linked to a light chain constant domain (C_{L}) (i.e., V_{L}1-V_{L}2-C_{L}); wherein the first polypeptide associates with the second polypeptide by a multimerizing component, and wherein a first third polypeptide molecule associates with the first polypeptide, and wherein a second third polypeptide molecule associates with the second polypeptide (see, e.g., FIG. 1B). In one embodiment, V_{L}1 and V_{L}2 are derived from two different immunoglobulin light chain V gene segments.

In one embodiment, M1 and M2 each independently comprise an immunoglobulin heavy chain constant domain or multimerizing fragment thereof. In one embodiment, the immunoglobulin heavy chain constant domain or multimerizing fragment thereof is human. In one embodiment, M1 and M2 each independently comprise an immunoglobulin heavy chain constant domain selected from C_{H}2, C_{H}3, and a combination thereof. In a specific embodiment, M1 and M2 each independently comprise a human C_{H}2 and C_{H}3, arranged, e.g., as found in a human Fc, e.g., in a human IgG1, IgG2, IgG3, or IgG4 Fc.

In one embodiment, M1 and M2 each independently comprise an immunoglobulin light chain constant domain or multimerizing fragment thereof. In one embodiment the immunoglobulin light chain constant domain or multimerizing fragment thereof is human. In one embodiment, M1 and M2 each independently comprise an immunoglobulin light chain constant domain selected from Cκ, Cλ and a combination thereof. In a specific embodiment, M1 and M2 each independently comprise a human Cκ. In a specific embodiment, M1 and M2 each independently comprise a human Cλ.

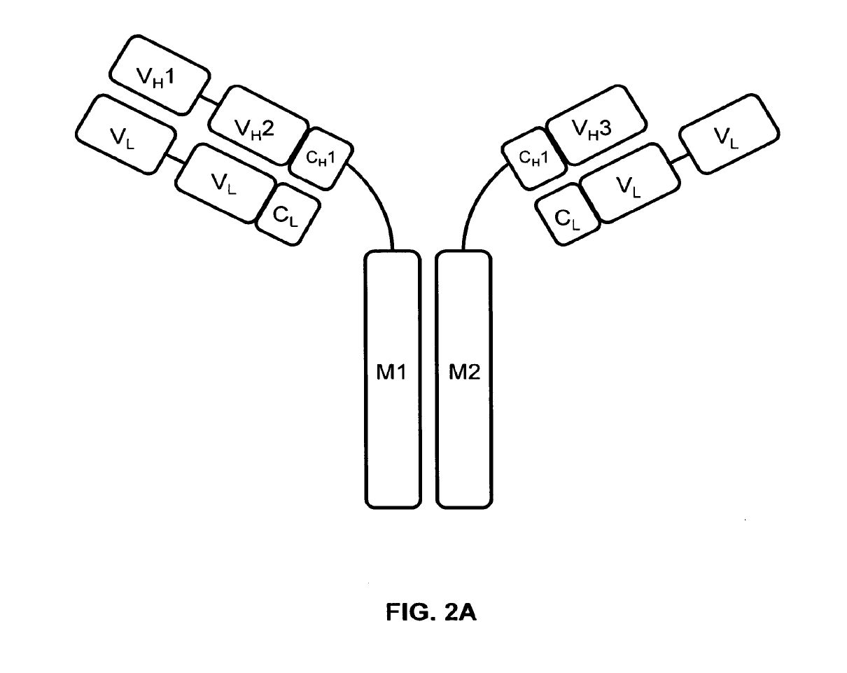

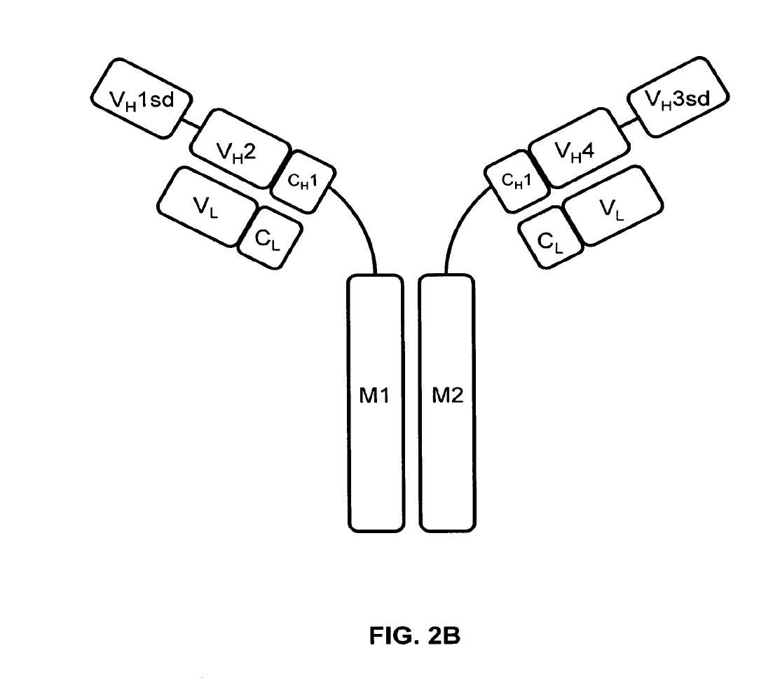

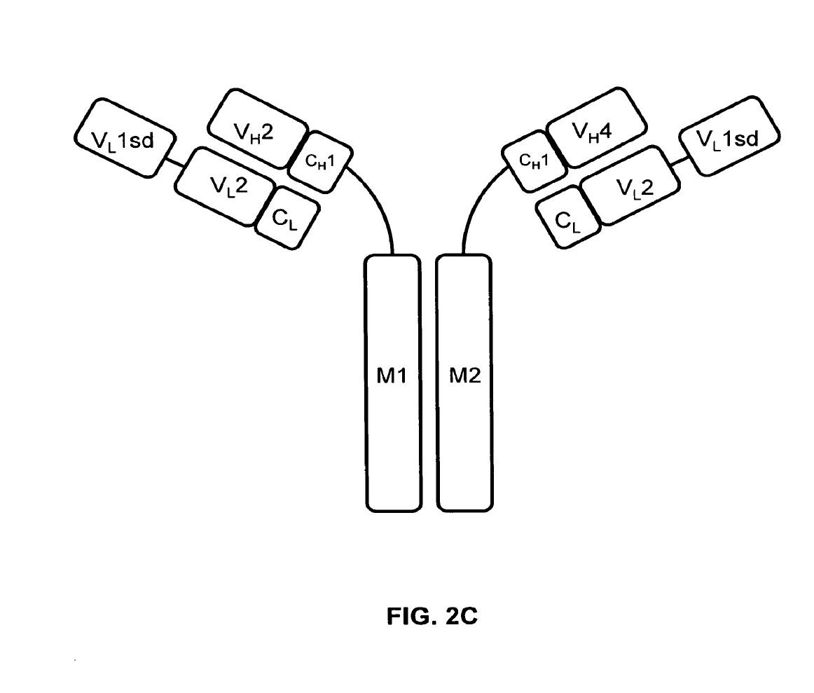

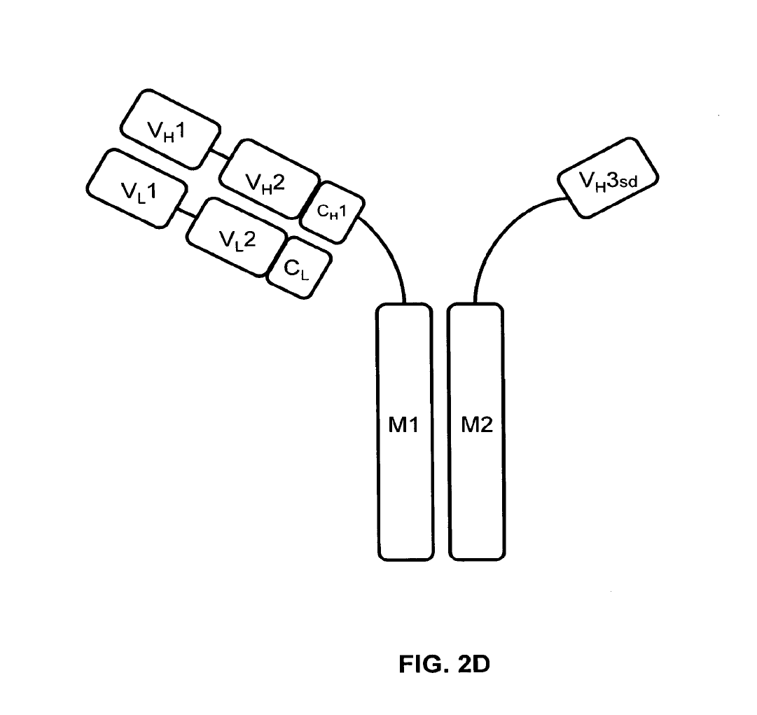

In one aspect, a multivalent antigen-binding protein is provided that comprises a first polypeptide comprising a first heavy chain variable domain (V_{H}1) fused directly or through a linker to a second heavy chain variable domain (V_{H}2) fused directly or through a linker to a C_{H}1 region that is attached directly or through a linker to a multimerizing component M1; a second polypeptide comprising a third heavy chain variable domain (V_{H}3) fused directly or through a linker to a C_{H}1 region that is attached directly or through a linker to a multimerizing component M2; and a third polypeptide that comprises a first light chain variable domain V_{L} fused directly or through a linker to a second light chain variable domain V_{L} that is fused directly or through a linker to a light chain constant region. The multivalent antigen-binding protein comprises a heterodimer of the first and the second polypeptide, wherein each of the first and the second polypeptide are each associated with one moiety of the third polypeptide. See, e.g., FIG. 2A.

In one embodiment, the multivalent antigen-binding protein consists essentially of three polypeptides, wherein the first polypeptide consists essentially of a first heavy chain immunoglobulin variable domain (V_{H}1) fused directly or through a linker to a second heavy chain immunoglobulin variable domain (V_{H}2) fused directly or through a linker to a C_{H}1 region that is attached directly or through a linker to a multimerizing component M1; a second polypeptide that consists essentially of a third heavy chain immunoglobulin variable domain (V_{H}3) fused directly or through a linker to a C_{H}1 region that is fused directly or through a linker to a multimerizing component M2; and a third polypeptide (present in two copies) consisting essentially of an immunoglobulin light chain variable domain fused directly or through a linker to a second immunoglobulin light chain variable domain, which is in turn fused directly or through a linker to a light chain constant region. In one embodiment, the first and the second immunoglobulin light chain constant domains are cognate to each of V_{H}1, V_{H}2, and V_{H}3, e.g., having been obtained from a non-human animal capable of expressing a light chain derived from a single light chain variable gene segment (e.g., a rearranged V/J gene), wherein each of V_{H}1, V_{H}2, and V_{H}3 are derived from the same non-human animal.

In one embodiment, the multivalent antigen-binding protein comprises a polypeptide comprising a tandem (optionally separated by a linker) pair of immunoglobulin light chain variable domains, one of which is fused (optionally through a linker) to a light chain constant region, wherein each of the pair of immunoglobulin light chain variable domains is cognate with a sequences of a second polypeptide having each of two different heavy chain variable domains V_{H}1 and V_{H}2, wherein one of the heavy chain variable domains is associated with a C_{H}1 region, and the second polypeptide is associated with a multimerizing component; and a third polypeptide comprising a third heavy chain variable domain associated with a C_{H}1 region and a multimerizing component, but not associated with a light chain variable domain. In one embodiment, the unassociated light chain variable domain is a single light chain variable domain that specifically binds an epitope that is not bound by V_{H}1 or V_{H}2, or that is not bound by V_{H}1 or V_{H}2 or V_{H}3.

In one embodiment, the multivalent antigen-binding protein comprises a first polypeptide that comprises (or that consists essentially of) a V_{H}1, a V_{H}2, a C_{H}1, an M1; a second polypeptide that comprises (or that consists essentially of) a V_{H}3, a C_{H}1, and a M2; a third polypeptide comprising an immunoglobulin light chain variable domain (V_{L}) present in two copies (in one embodiment separated by a linker sequence), and an immunoglobulin light chain constant domain (C_{L}) V_{L}-V_{L}-C_{L}); wherein the first polypeptide associates with the second polypeptide by multimerizing components M1 and M2, and wherein one third polypeptide molecule associates with the first polypeptide, and wherein one third polypeptide molecule associates with the second polypeptide (see, e.g., FIG. 2A).

In an alternate embodiment, the antigen-binding protein comprises one copy of the third polypeptide and one copy of a fourth polypeptide, wherein the fourth polypeptide comprises (or consists essentially of) an immunoglobulin light chain constant domain (C_{L}) that is derived from the same light chain V segment as the C_{L} of the third polypeptide. In a specific embodiment, the V_{L} of the third polypeptide is identical to the V_{L} of the fourth polypeptide.

In one aspect, a multivalent antigen-binding protein is provided, comprising a first polypeptide that comprises an immunoglobulin single heavy chain variable domain (V_{H}1sd) fused directly or through a linker to a second immunoglobulin heavy chain variable domain (V_{H}2), fused directly or through a linker to a C_{H}1 region, which is fused directly or through a linker or hinge to a multimerizing component M1 (e.g., from N-terminal to C-terminal V_{H}1sd-V_{H}2-C_{H}1-M1); a second polypeptide comprising a third immunoglobulin single heavy chain variable domain (V_{H}3sd) fused directly or through a linker to a fourth immunoglobulin heavy chain variable domain (V_{H}4) that is fused directly or through a linker to a C_{H}1 region, which is fused directly or through a linker to a multimerizing component M2; and a third polypeptide comprising a light chain variable domain fused directly or through a linker to a light chain constant region. In one embodiment, the light chain polypeptide consists essentially of a light chain variable domain and a light chain constant region. In one embodiment, the multivalent antigen-binding protein is the protein depicted in FIG. 2B.

In one embodiment, the V_{H}1sd and V_{H}3sd are made in a mouse that lacks a C_{H}1 and/or C_{H}1 and hinge in an IgG. In one embodiment, the V_{H}2 and the V_{H}4 are made in a mouse that expresses a single rearranged human light chain variable domain, and the V_{L} is the single rearranged human light chain variable domain; in a specific embodiment, the single rearranged human light chain variable domain is derived from a Vκ1-39 gene segment or a Vκ3-20 gene segment; in a specific embodiment the single rearranged human light chain variable domain is derived from a Vλ1-40 gene segment or a Vλ2-14 gene segment.

In one aspect, a multivalent antigen-binding protein is provided that comprises four antigen-binding moieties, wherein the first antigen-binding moiety is on a first polypeptide and is an unpaired single heavy chain variable domain fused directly or through a linker to a heavy chain immunoglobulin variable domain fused with a C_{H}1 domain fused to a multimerizing component M1, wherein the heavy chain immunoglobulin variable domain is associated with a cognate light chain variable domain associated with a light chain constant region, wherein the heavy chain variable domain and the cognate light chain variable domain comprise the second antigen-binding moiety; and wherein the third antigen-binding moiety is on a second polypeptide and comprises a single heavy chain immunoglobulin variable domain, wherein the third antigen-binding moiety is fused with a fourth heavy chain immunoglobulin variable domain fused with a C_{H}1 region and a multimerizing component M2, wherein the fourth heavy chain immunoglobulin variable domain forms the fourth antigen-binding domain in conjunction with a light chain variable domain fused with a light chain constant domain.

In one aspect, an antigen-binding protein is provided that comprises an antibody or antigen-binding fragment thereof, wherein the antibody further comprises a first single heavy chain immunoglobulin domain fused directly or through a linker to a first heavy chain immunoglobulin domain of the antibody (which is cognate with a light chain variable domain), and comprises a second single heavy chain immunoglobulin domain fused directly or through a linker to the second heavy chain immunoglobulin domain of the antibody (which is also cognate with a light chain variable domain).

In one aspect, an antigen-binding protein is provided that comprises two single domain heavy chain immunoglobulin antigen-binding domains that bind two different epitopes, and two cognate pairs of immunoglobulin heavy and light chain variable domains, wherein the two cognate pairs of immunoglobulin heavy and light chain variable domains each bind the same or a different antigen. See, e.g., FIG. 2B.

In one aspect, a multivalent antigen-binding protein is provided that comprises a first polypeptide comprising (or consisting essentially of) a first immunoglobulin heavy chain single variable domain (V_{L}1sd) fused directly or through a linker to a light chain variable domain (V_{L}2), which is fused directly or through a linker to a light chain constant region; a second polypeptide comprising (or consisting essentially of) a second heavy chain variable domain (V_{H}2) fused directly or through a linker to a C_{H}1 region, which is fused directly or through a linker to a first multimerizing component M1 (e.g., from N-terminal to C-terminal, V_{H}2-C_{H}1-M1); and a third polypeptide comprising (or consisting essentially of) a fourth heavy chain variable domain (V_{H}4) fused directly or through a linker to a C_{H}1 region that is fused directly or through a linker to a second multimerizing component M2 (e.g., from N-terminal to C-terminal V_{H}4-C_{H}1-M2). See, e.g., FIG. 2C.

In one embodiment, the V_{L}1sd is made in a mouse that lacks a C_{H}1 and/or lacks a hinge region in an IgG. In one embodiment, the V_{H}2 and the V_{H}4 are made in a mouse that expresses a single light chain derived from a single germline rearranged light chain gene; in one embodiment, the single rearranged human light chain variable domain is derived from a Vκ1-39 gene segment or a Vκ3-20 gene segment; in one embodiment, the single rearranged human light chain variable domain is derived from a Vλ1-40 gene segment or a Vλ2-14 gene segment.

In one aspect, an antigen-binding protein is provided that comprises two pairs of cognate heavy and light chain variable domains, wherein each of the two pairs of cognate heavy and light chain variable domains bind the same or a different antigen; and comprises two single (non-cognate) light chain variable domains that each bind the same or a different antigen; wherein the heavy chain variable domains are associated with C_{H}1 region, and wherein each C_{H}1 region is associated with a multimerizing component.

In one embodiment, the antigen-binding protein consists essentially of two pairs of cognate heavy and light chain variable domains, wherein each of the two pairs of cognate heavy and light chain variable domains bind the same or a different antigen; and consists essentially of two single (non-cognate) light chain variable domains that each bind the same or a different antigen; wherein the first cognate heavy chain variable domain is associated with a first C_{H}1 region that is associated with a first multimerizing component M1, and the second cognate heavy chain variable domain is associated with a second C_{H}1 region that is associated with a second multimerizing component M2. See, e.g., FIG. 2C.

In one aspect, a multivalent antigen-binding protein is provided, comprising a first polypeptide that comprises a first immunoglobulin heavy chain variable domain fused directly or through a linker to a second immunoglobulin heavy chain variable domain, which is fused directly or through a linker to a C_{H}1 region, which is attached directly or through a linker to a first multimerizing component (e.g., V_{H}1-V_{H}2-C_{H}1-M1); a second polypeptide comprising (or consisting essentially of) a heavy chain single immunoglobulin variable domain (V_{H}3sd) fused directly or through a linker to a second multimerizing component (e.g., V_{H}3sd-M2); and a third polypeptide comprising (or consisting essentially of) a first light chain variable domain fused directly or through a linker to a second immunoglobulin light chain variable domain (V_{L}2), which is fused directly or through a linker to a light chain constant domain (e.g., from N-terminal to C-terminal, V_{L}1-V_{L}2-C_{L}). See, e.g., FIG. 2D.

In one embodiment, the single immunoglobulin heavy chain variable domain (VH3sd) is made in a mouse that lacks a C_{H}1 or lacks a C_{H}1 and a hinge of an IgG. In one embodiment, the V_{H}1 and V_{H}2 are different from each other, and the V_{L}1 and V_{L}2 are different from each other.

In one embodiment, the V_{H}1 and V_{H}2 are different from each other, and the V_{L}1 and V_{L}2 are each derived from a single rearranged light chain variable gene in the germline of a non-human animal, wherein the V_{H}1 and the V_{H}2 are each made in the same non-human animal that expresses only a single rearranged light chain derived from a single rearranged light chain gene in the germline of the non-human animal (i.e., the V_{L}1 and the V_{L}2 are derived from the same rearranged sequence).

In one aspect, a multivalent antigen-binding protein is provided that comprises a first polypeptide consisting essentially of a first multimerizing component (M1 in FIG. 2D) and a single immunoglobulin binding domain (e.g., a V_{H}sd or a V_{L}sd); a second polypeptide consisting essentially of a second multimerizing component (M1 in FIG. 2D) associated with a C_{H}1 region which is associated with a heavy chain immunoglobulin variable domain (e.g., V_{H}2 in FIG. 2D) fused directly or through a linker to a second heavy chain immunoglobulin variable domain (e.g., V_{H}1 in FIG. 2D), wherein the first and the second heavy chain immunoglobulin variable domains are associated with cognate light chain variable domains, and wherein the first cognate heavy chain constant domain (C_{H}1 in FIG. 2D) is associated with a light chain constant domain (C_{L} in FIG. 2D).

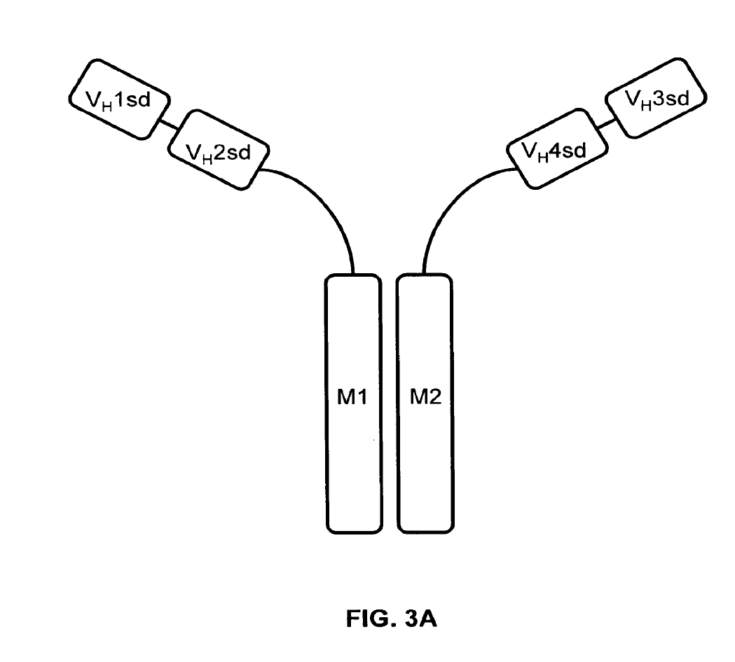

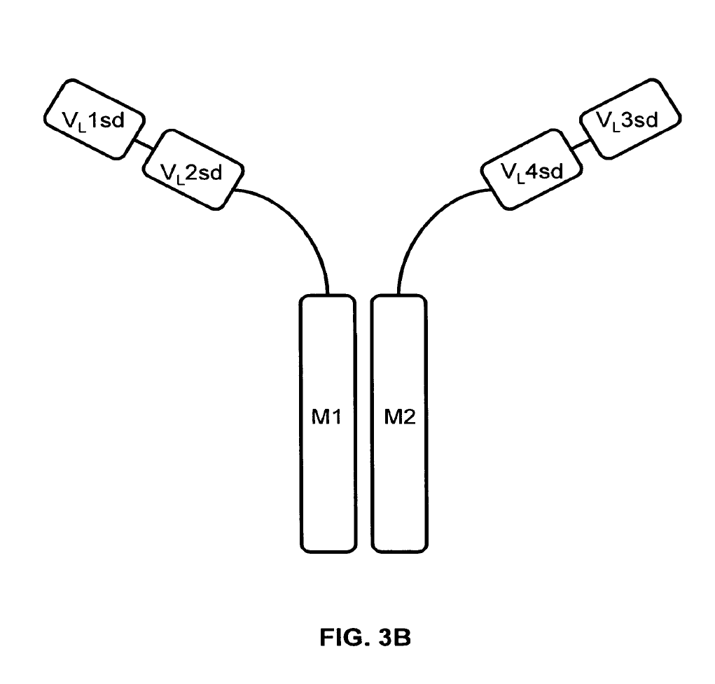

In one aspect, a multivalent antigen-binding protein is provided, comprising (or consisting essentially of) a first polypeptide that comprises a first single immunoglobulin heavy chain variable domain (V_{H}1sd), a second heavy chain single immunoglobulin variable domain (V_{H}2sd), and a first multimerizing component (M1) (i.e., V_{H}1sd-V_{H}2sd-M1), wherein the first polypeptide lacks an immunoglobulin C_{H}1 domain; a second polypeptide that comprises a third single immunoglobulin heavy chain variable domain (V_{H}3sd), a fourth immunoglobulin heavy chain variable domain (V_{H}4sd) and a second multimerizing component (M2) (i.e., V_{H}3sd-V_{H}4sd-M2), wherein the second polypeptide lacks an immunoglobulin C_{H}1 domain; wherein the first polypeptide associates with the second polypeptide by a multimerizing component (see, e.g., FIG. 3A).

In one embodiment, each of the heavy chain single immunoglobulin variable domains is made in a non-human animal that lacks a C_{H}1 in an IgG gene. In one embodiment, the antigen-binding protein consists essentially of two polypeptides, wherein the first polypeptide consists essentially of two heavy chain single immunoglobulin variable domains (V_{H}1sd and V_{H}2sd) and a multimerizing component; and wherein the second polypeptide consists essentially of two heavy chain single immunoglobulin variable domains (V_{H}3sd and V_{H}4sd). In one embodiment, V_{H}1sd, V_{H}2sd, V_{H}3sd, and V_{H}4sd bind to at least one, at least two, at least three, or four epitopes. In one embodiment, the antigen-binding protein binds at least two antigens; in one embodiment, the antigen-binding protein binds at least three antigens; in one embodiment, the antigen-binding protein binds four antigens.

In one aspect, a multivalent binding protein is provided that comprises (or consists essentially of) two polypeptides, wherein the first polypeptide comprises (or consists essentially of) a first single light chain variable domain (V_{L}1sd) attached directly or through a linker to a second single light chain variable domain (V_{L}2sd) that is linked directly or through a linker to a first multimerizing component M1, and the second polypeptide comprises (or consists essentially of) a third light chain variable domain (V_{L}3sd) attached directly or through a linker to a fourth single light chain variable domain (V_{L}4sd) that is linked directly or through a linker to a second multimerizing component M2.

In one embodiment, an antigen-binding protein is provided consisting essentially of two polypeptides, wherein the first polypeptide consists essentially of a first multimerizing component M1 that comprises a first and a second single immunoglobulin heavy chain variable domain, and the second polypeptide consists essentially of a second multimerizing component and a third and a fourth single immunoglobulin heavy chain variable domain. In one embodiment, the first, second, third, and fourth single immunoglobulin heavy chain variable domain each binds a different antigen. In one embodiment, the first, second, third, and fourth single immunoglobulin heavy chain variable domains bind a total of three antigens. See, e.g., FIG. 3A.

In one aspect, a multivalent antigen-binding protein is provided, comprising four single light chain variable domains and two multimerizing components, wherein each multimerizing component comprises at least one single light chain variable domain. In one aspect, a multivalent antigen-binding protein is provided that comprises a first multimerizing component M1 that comprises two single light chain immunoglobulin variable domains, and a second multimerizing component M2 that comprises one or two single light chain immunoglobulin variable domains. In one embodiment, the multivalent antigen-binding protein consists essentially of two polypeptides, wherein the first polypeptide consists essentially of a first multimerizing component M1 associated (e.g., fused) with a first single domain light chain immunoglobulin variable domain (e.g., V_{L}2sd in FIG. 3B) that is attached directly or through a linker a second single domain light chain immunoglobulin variable domain (e.g., V_{L}1sd in FIG. 3B); and the second polypeptide consists essentially of a second multimerizing component M2 fused (directly or through a linker) to yet another single domain light chain variable domain (e.g., V_{L}4sd in FIG. 3B) that is fused directly or through a linker to yet another single domain light chain variable domain (e.g., V_{L}3sd in FIG. 3B). In various embodiments, the single light chain variable domains bind an antigen in the absence of a cognate heavy chain, and, e.g., are made in a non-human animal with a restricted heavy chain repertoire (e.g., in a mouse or rat that makes heavy chains derived from a repertoire of just a single heavy chain variable gene segment (and, e.g., a D segment and a J segment), and thus the light chain variable domains bind a target antigen in the absence of a cognate heavy chain.

In one aspect, a multivalent antigen-binding protein is provided, comprising a first polypeptide that comprises (or consists essentially of) a first immunoglobulin heavy chain variable domain (V_{H}1), a second heavy chain immunoglobulin variable domain (V_{H}2), and a first multimerizing component (M1) (i.e., V_{H}1-V_{H}2-M1), wherein the first polypeptide lacks an immunoglobulin C_{H}1 domain; a second polypeptide that comprises (or consists essentially of) a third immunoglobulin heavy chain variable domain (V_{H}3), and a second multimerizing component (M2) (i.e., V_{H}3-V_{H}4-C_{H}1-M2), wherein the second polypeptide lacks an immunoglobulin C_{H}1 domain; wherein the first polypeptide associates with the second polypeptide by a multimerizing component. In one embodiment, the multivalent antigen-binding protein lacks an immunoglobulin light chain variable domain and lacks an immunoglobulin light chain constant domain.

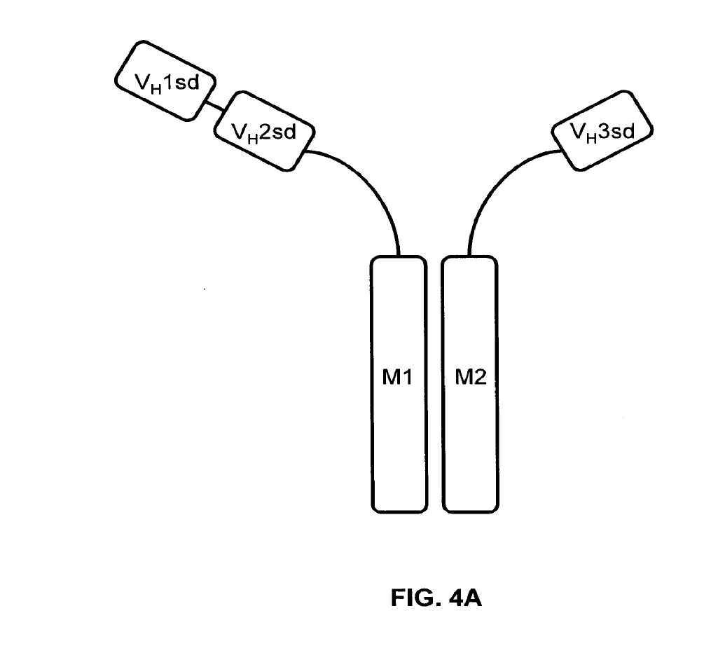

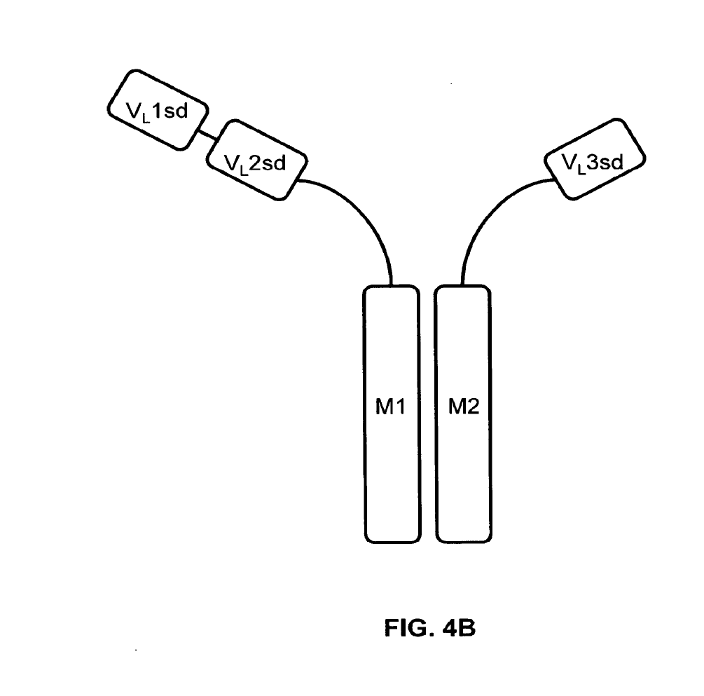

In one aspect, a multivalent antigen-binding protein is provided that binds three different antigens by three different heavy chain immunoglobulin single variable domains. In one embodiment, a first and a second heavy chain immunoglobulin single variable domain are disposed on a first multimerizing component M1, and the third heavy chain immunoglobulin single variable domain is disposed on a second multimerizing component M2. In one embodiment, the antigen-binding protein consists essentially of a first polypeptide and a second polypeptide, wherein the first polypeptide consists essentially of a first heavy chain immunoglobulin single variable domain (V_{H}1sd) fused directly or through a linker to a second heavy chain immunoglobulin singe variable domain (V_{H}2sd) fused directly or through a linker to a first multimerizing component M1; and the second polypeptide consists essentially of a third heavy chain immunoglobulin single variable domain (V_{H}3sd) fused directly or through a linker to a second multimerizing component M2. In one embodiment, the first, the second, and the third single variable domain bind three different antigens. In one embodiment, the first, the second, and the third single variable domains bind a total of two antigens. See, e.g., FIG. 4A.

In one aspect, a multivalent antigen-binding protein is provided, comprising a first polypeptide that comprises a first immunoglobulin heavy chain single variable domain (V_{H}1sd), a second heavy chain immunoglobulin single variable domain (V_{H}2sd), and a first multimerizing component (M1) V_{H}1sd-V_{H}2sd-M1), wherein the first polypeptide lacks an immunoglobulin C_{H}1 domain; a second polypeptide that comprises a third immunoglobulin heavy chain single variable domain (V_{H}3sd), and a second multimerizing component (M2) (i.e., V_{H}3-V_{H}4-M2), wherein the second polypeptide lacks an immunoglobulin C_{H}1 domain; wherein the first polypeptide associates with the second polypeptide by a multimerizing component. See, e.g., FIG. 4B.

In one embodiment, the first and the second immunoglobulin heavy chain variable domains are single immunoglobulin heavy chain variable domains (V_{H}1sd and V_{H}2sd, respectively). In one embodiment, the third and the fourth immunoglobulin heavy chain variable domains are single immunoglobulin heavy chain variable domains (V_{H}3sd and V_{H}4sd, respectively). In one embodiment, the multivalent antigen-binding protein comprises a first polypeptide comprising a first single immunoglobulin heavy chain variable domain (V_{H}1sd) linked directly or via a linker to a second single immunoglobulin heavy chain variable domain (V_{H}2sd) that is linked to directly or via a linker to a first multimerizing component (M1); and a second polypeptide comprising a third single immunoglobulin heavy chain variable domain (V_{H}3sd) linked directly or via a linker to a fourth single immunoglobulin heavy chain variable domain (V_{H}4sd) linked directly or via a linker to a second multimerizing component (M2) (see, e.g., FIG. 3A).

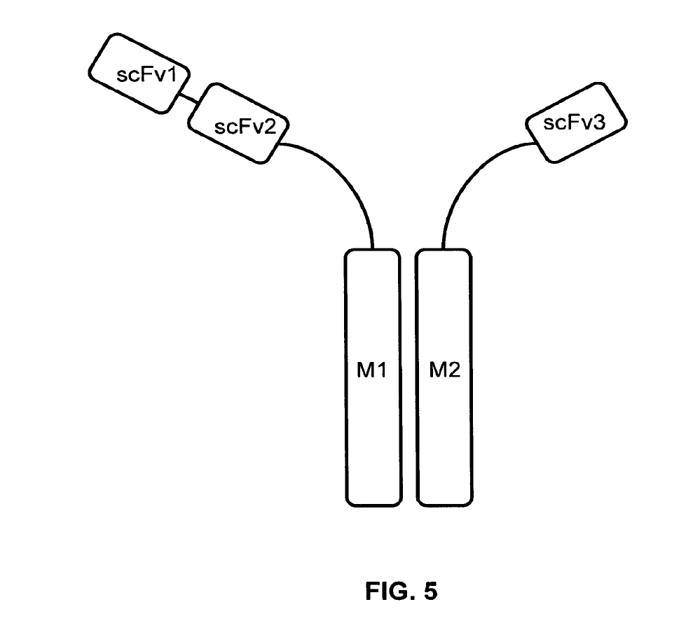

In one aspect, a multivalent antigen-binding protein is provided, comprising a first polypeptide that comprises a first scFv (scFv1), a second scFv (scFv2), optionally a linker, and a first multimerizing component (M1); and a second polypeptide that comprises a third scFv (scFv3), optionally a linker, and a second multimerizing component (M2) (see, e.g., FIG. 5).

In one embodiment, the multivalent antigen-binding protein consists essentially of a first polypeptide and a second polypeptide, wherein the first polypeptide consists essentially of a first scFv (scFv1) fused directly or through a linker to a second scFv (scFv2), which is fused directly or through a linker to a first multimerizing component (M1); and wherein the second polypeptide consists essentially of a third scFv (scFv3) fused directly or through a linker to a second multimerizing component M2.

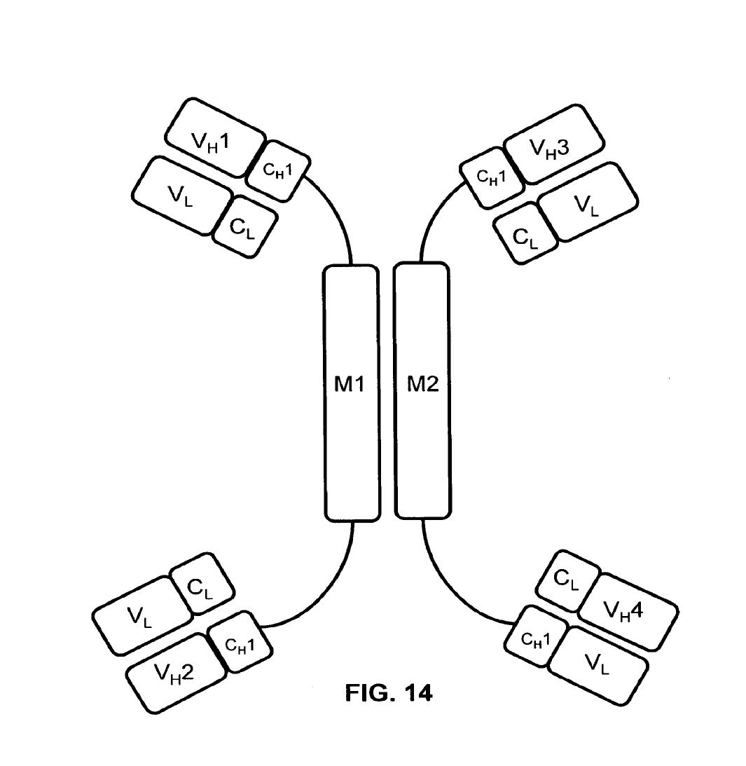

In one aspect, a multivalent antigen-binding protein is provided, comprising a first polypeptide that comprises a first immunoglobulin heavy chain variable domain (V_{H}1), a heavy chain C_{H}1 constant domain (C_{H}1), a first multimerizing component (M1), a second C_{H}1, a second heavy chain immunoglobulin variable domain (V_{H}2) (i.e., V_{H}1-C_{H}1-M1-C_{H}1-V_{H}2); a second polypeptide that comprises a third immunoglobulin heavy chain variable domain (V_{H}3), a C_{H}1, a second multimerizing component (M2), a C_{H}1, and a fourth immunoglobulin heavy chain variable domain (C_{H}4); a third polypeptide comprising an immunoglobulin light chain variable domain (V_{L}) and an immunoglobulin light chain constant domain (C_{L}) (i.e., V_{L}-C_{L}); wherein the first polypeptide associates with the second polypeptide by multimerizing components M1 and M2, and wherein two third polypeptide molecules associate with the first polypeptide, and wherein two third polypeptide molecules associates with the second polypeptide (see, e.g., FIG. 14).

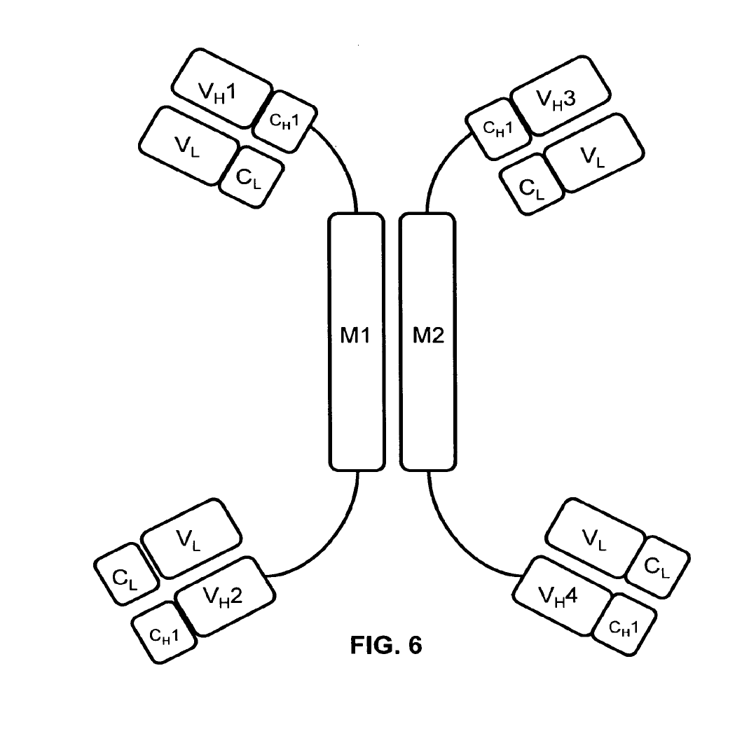

In one aspect, a multivalent antigen-binding protein is provided, comprising a first polypeptide that comprises a first immunoglobulin heavy chain variable domain (V_{H}1), a heavy chain C_{H}1 constant domain (C_{H}1), a first multimerizing component (M1), a second C_{H}1, a second heavy chain immunoglobulin variable domain (V_{H}2) (i.e., V_{H}1-C_{H}1-M1-V_{H}2-C_{H}1); a second polypeptide that comprises a third immunoglobulin heavy chain variable domain (V_{H}3), a C_{H}1, a second multimerizing component (M2), a C_{H}1, and a fourth immunoglobulin heavy chain variable domain (C_{H}4); a third polypeptide comprising an immunoglobulin light chain variable domain (V_{L}) and an immunoglobulin light chain constant domain (C_{L}) V_{L}-C_{L}); wherein the first polypeptide associates with the second polypeptide by multimerizing components M1 and M2, and wherein two third polypeptide molecules associate with the first polypeptide, and wherein two third polypeptide molecules associates with the second polypeptide (see, e.g., FIG. 6).

In one embodiment, the multivalent antigen-binding protein consists essentially of six polypeptides, wherein the first polypeptide consists essentially of (from N-terminal to C-terminal), a first immunoglobulin heavy chain variable domain (V_{H}1) fused directly or through a linker to a first C_{H}1, which is fused directly or through a linker to a first multimerizing component (M1), which is fused directly or through a linker to a second immunoglobulin heavy chain variable domain (V_{H}2) which is in turn fused directly or through a linker to a second C_{H}1 region. The V_{H}1-C_{H}1 region is associated with a second polypeptide that consists essentially of a cognate (with respect to V_{H}1) immunoglobulin light chain variable domain fused directly or through a linker to a light chain constant domain; the V_{H}2-C_{H}1 region is associated with a third polypeptide that consists essentially of a cognate (with respect to V_{H}2) immunoglobulin light chain domain fused directly or through a linker to a light chain constant domain. The fourth polypeptide consists essentially of (from N-terminal to C-terminal) a third immunoglobulin heavy chain variable domain (V_{H}3) fused directly or through a linker to a C_{H}1 region, which is in turn fused directly or through a linker to a second multimerizing component M2, which is fused directly or through a linker to a fourth immunoglobulin heavy chain variable domain (V_{H}4) fused directly or through a linker to a C_{H}1 region; wherein a fifth polypeptide consisting essentially of an immunoglobulin light chain variable domain that is cognate with respect to V_{H}3 fused directly or through a linker to a light chain constant region is associated with V_{H}3; and wherein a sixth polypeptide consisting essentially of an immunoglobulin light chain variable domain that is cognate with respect to V_{H}4 is fused directly or through a linker to a light chain constant region and is associated with V_{H}4. In one embodiment, one or more of V_{H}1, V_{H}2, V_{H}3, and V_{H}4 are made in a non-human animal that expresses a single immunoglobulin light chain variable domain from a single rearranged (V/J) light chain variable region gene in the germline of the animal, and the cognate light chain variable domains of the antigen-binding protein are derived from the same rearranged light chain variable region. In a specific embodiment, the rearranged light chain variable region gene is derived from a Vκ1-39/J rearrangement or a Vκ3-20/J rearrangement. In a specific embodiment, the rearranged light chain variable region is derived from a Vλ1-40/J rearrangement or a Vλ2-14/J rearrangement. See, e.g., FIG. 6.

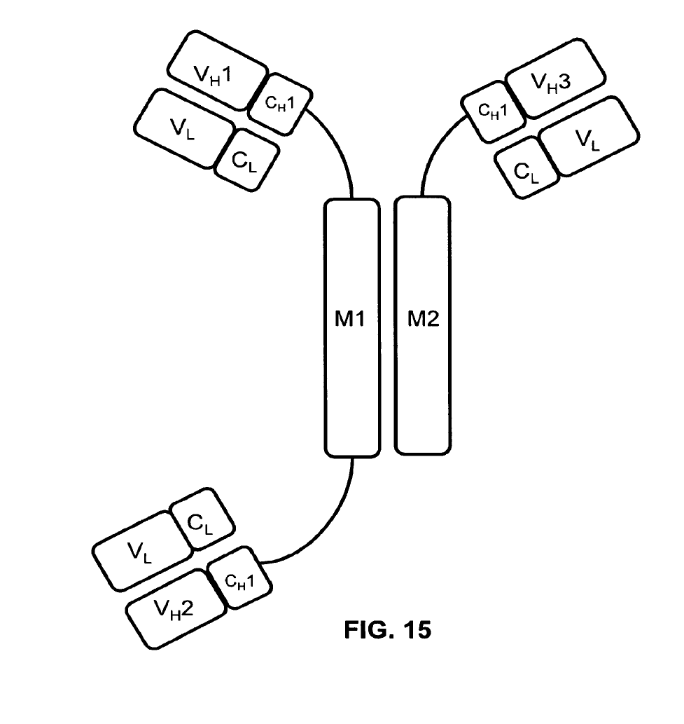

In one aspect, a multivalent antigen-binding protein is provided, comprising a first polypeptide that comprises a first immunoglobulin heavy chain variable domain (V_{H}1), a heavy chain C_{H}1 constant domain (C_{H}1), a first multimerizing component (M1), a second C_{H}1, a second heavy chain immunoglobulin variable domain (V_{H}2) (i.e., V_{H}1-C_{H}1-M1-C_{H}1-V_{H}2); a second polypeptide that comprises a third immunoglobulin heavy chain variable domain (V_{H}3), a C_{H}1, and a second multimerizing component (M2); a third polypeptide comprising an immunoglobulin light chain variable domain (V_{L}) and an immunoglobulin light chain constant domain (C_{L}) (i.e., V_{L}-C_{L}); wherein the first polypeptide associates with the second polypeptide by multimerizing components M1 and M2, and wherein two third polypeptide molecules associate with the first polypeptide, and wherein one third polypeptide molecule associates with the second polypeptide (see, e.g., FIG. 15).

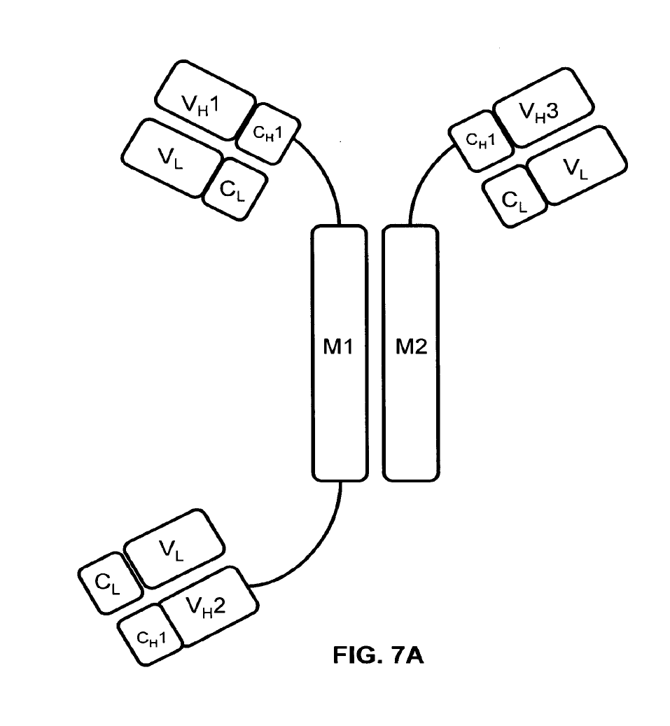

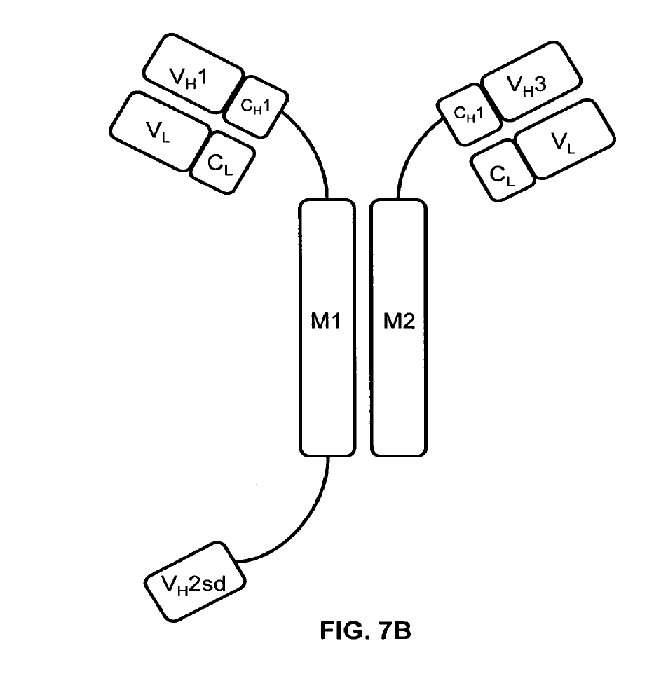

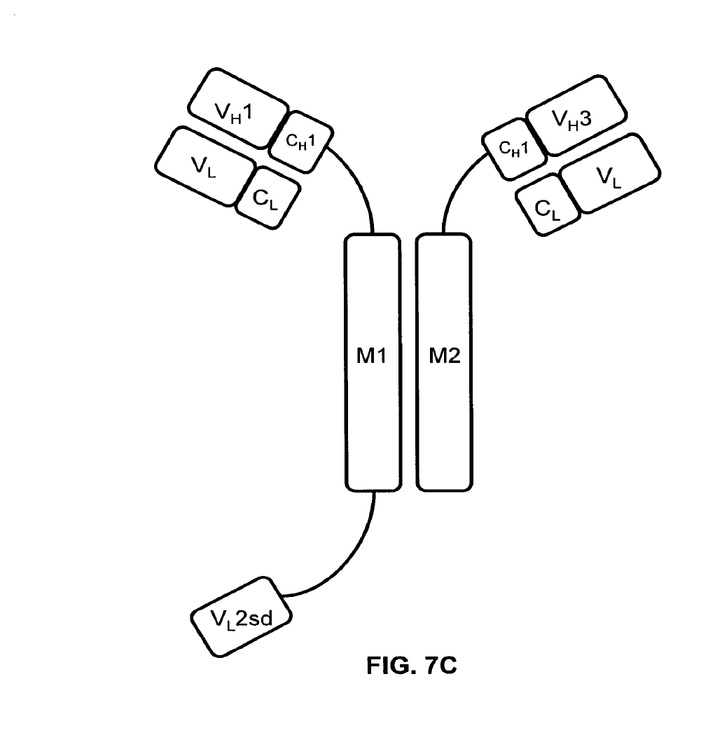

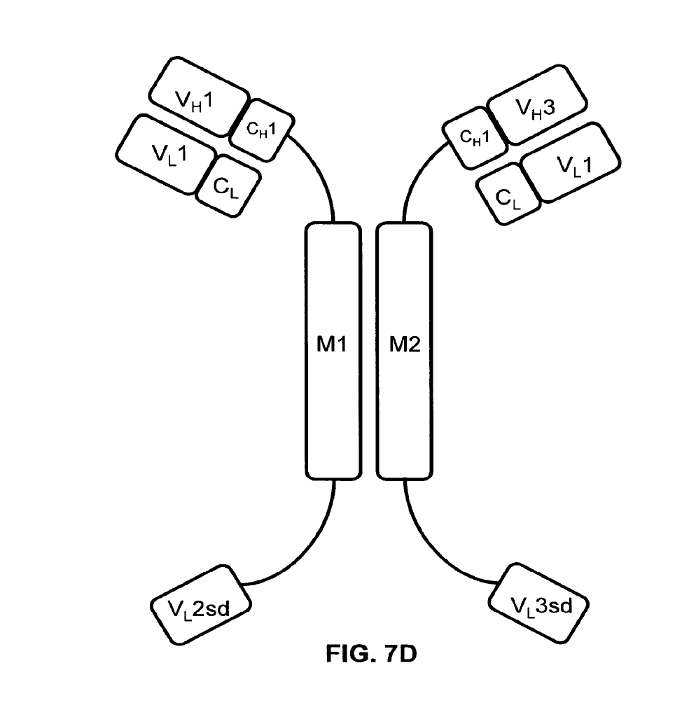

In one aspect, a multivalent antigen-binding protein is provided, comprising a first polypeptide that comprises a first immunoglobulin heavy chain variable domain (V_{H}1), a heavy chain C_{H}1 constant domain (C_{H}1), a first multimerizing component (M1), a second C_{H}1, a second heavy chain immunoglobulin variable domain (V_{H}2) (i.e., V_{H}1-C_{H}1-M1-V_{H}2-C_{H}1); a second polypeptide that comprises a third immunoglobulin heavy chain variable domain (V_{H}3), a C_{H}1, and a second multimerizing component (M2); a third polypeptide comprising an immunoglobulin light chain variable domain (V_{L}) and an immunoglobulin light chain constant domain (C_{L}) (i.e., V_{L}-C_{L}); wherein the first polypeptide associates with the second polypeptide by multimerizing components M1 and M2, and wherein two third polypeptide molecules associate with the first polypeptide, and wherein one third polypeptide molecule associates with the second polypeptide (see, e.g., FIG. 7A).

In one embodiment, the antigen-binding protein consists essentially of five polypeptides, wherein the first polypeptide consists essentially of (from N-terminal to C-terminal) a first heavy chain immunoglobulin variable domain (V_{H}1) fused directly or through a linker to a first C_{H}1, which is in turn fused directly or through a linker to a first multimerizing component M1, which is fused directly or through a linker to a second heavy chain immunoglobulin variable domain that is fused directly or through a linker to a second C_{H}1 sequence; a second polypeptide that consists essentially of (from N-terminal to C-terminal) a third immunoglobulin heavy chain variable domain (V_{H}3) fused directly or through a linker to a third C_{H}1 region, which in turn is fused directly or through a linker to a second multimerizing component M2; a third polypeptide that consists essentially of a first light chain variable domain that is cognate with V_{H}1 and that is fused directly or through a linker to a first light chain constant region, wherein the third polypeptide is associated with V_{H}1-C_{H}1; a fourth polypeptide that consists essentially of a second light chain variable domain that is cognate with V_{H}2, and that is fused directly or through a linker to a second light chain constant region, wherein the fourth polypeptide is associated with V_{H}2-C_{H}1; and a fifth polypeptide that consists essentially of a third light chain variable domain that is cognate with V_{H}3, and that is fused directly or through a linker to a third light chain constant region, wherein the fifth polypeptide is associated with V_{H}3-C_{H}1. In one embodiment, V_{H}1, V_{H}2, and V_{H}3 are derived from a non-human animal that expresses a single immunoglobulin light chain from a single rearranged (V/J) light chain gene in the germline of the non-human animal, and each of the first, the second, and the third light chain variable domains are derived from that same rearranged light chain. In one embodiment, V_{H}1, V_{H}2, and V_{H}3 each specifically bind a different antigen. In one embodiment, V_{H}1, V_{H}2, and V_{H}3 bind no more than two antigens (e.g., two or more of V_{H}1, V_{H}2, and V_{H}3 bind different epitopes of a same antigen). See, e.g., FIG. 7A.

In one aspect, a multi-specific antigen-binding protein is provided that comprises three polypeptides, wherein the first polypeptide comprises (from N-terminal to C-terminal) a first heavy chain immunoglobulin variable domain (V_{H}1) fused directly or through a linker to a first C_{H}1 region, which in turn is fused directly or through a linker to a first multimerizing component M1, which is in turn fused directly or through a linker to a second heavy chain immunoglobulin variable domain that is a heavy chain single immunoglobulin variable domain (V_{H}2sd); a second polypeptide comprising (from N-terminal to C-terminal) a third heavy chain immunoglobulin variable domain (V_{H}3) fused directly or through a linker to a C_{H}1 region, which in turn is fused directly or through a linker to a second multimerizing component; a third polypeptide comprising a first light chain variable domain that is cognate with V_{H}1, wherein the first light chain variable domain is fused directly or through a linker to a light chain constant domain; and a fourth polypeptide comprising a second light chain variable domain that is cognate with V_{H}3, wherein the second light chain variable domain is fused directly or through a linker to a second light chain constant domain. See, e.g., FIG. 7B.

In one embodiment, the antigen-binding protein consists essentially of three polypeptides, wherein the first polypeptide consists essentially of first heavy chain immunoglobulin variable domain V_{H}1 fused directly or through a linker to a first C_{H}1 region, which in turn is fused directly or through a linker to a first multimerizing component M1, which is in turn fused directly or through a linker to a second heavy chain immunoglobulin variable domain that is a heavy chain single immunoglobulin variable domain (V_{H}2sd); and a second polypeptide that consists essentially of a third heavy chain immunoglobulin variable domain fused directly or through a linker to a C_{H}1, which in turn is fused directly or through a linker to a second multimerizing component M2; and a third polypeptide that consists essentially of a light chain variable domain that is cognate with V_{H}1, and is associated with a light chain constant region; and a fourth polypeptide that consists essentially of a light chain variable domain that is cognate with V_{H}3, and is associated with a light chain constant region. See, e.g., FIG. 7B.

In one embodiment, V_{H}1 and V_{H}3 are derived from a non-human animal that expresses a single immunoglobulin light chain variable domain from a single rearranged (V/J) light chain variable gene in the germline of the non-human animal, and the V_{L} that is cognate with V_{H}1 and the V_{L} that is cognate with V_{H}3 are derived from the same single rearranged light chain variable gene; and wherein V_{H}2sd is a variable domain obtained from a non-human animal that lacks a C_{H}1 gene or that lacks a C_{H}1 gene and a hinge sequence gene in an IgG. See, e.g., FIG. 7B. In a specific embodiment, the single rearranged light chain gene is derived from a Vκ1-39/J or a Vκ3-20/J rearranged gene, and the constant region is a κ constant region. In a specific embodiment, the single rearranged light chain gene is derived from a Vλ1-40/J or a Vλ2-14/J rearranged gene, and the constant region is a λ constant region.

In one aspect, a multivalent antigen-binding protein is provided comprising four polypeptides, wherein the first polypeptide comprises (from N-terminal to C-terminal) a first heavy chain immunoglobulin variable domain (V_{H}1) fused directly or through a linker to a first C_{H}1 sequence, which is fused directly or through a linker to a first multimerizing component M1, which in turn is fused with a light chain immunoglobulin single variable domain (V_{L}2sd); a second polypeptide (from N-terminal to C-terminal) comprising a third immunoglobulin variable domain that is a heavy chain immunoglobulin variable domain (V_{H}3) fused directly or through a linker to a second C_{H}1 region, which in turn is fused directly or through a linker to a second multimerizing component M2; a third polypeptide that comprises a light chain variable domain (V_{L}) that is cognate with V_{H}1, and that is associated with a light chain constant region (C_{L}); and a fourth polypeptide that comprises a light chain variable domain (V_{L}) that is cognate with V_{H}3, and that is associated with a light chain variable domain. See, e.g., FIG. 7C.

In one embodiment, the multivalent antigen-binding protein consists essentially of four polypeptides, wherein the first polypeptide consists essentially of a first heavy chain immunoglobulin variable domain (V_{H}1) associated with a first C_{H}1 sequence, which is fused directly or through a linker to a first multimerizing component M1, which in turn is fused with a light chain immunoglobulin single variable domain (V_{L}2sd); a second polypeptide consisting essentially of (from N-terminal to C-terminal) a third immunoglobulin variable domain that is a heavy chain immunoglobulin variable domain (V_{H}3) fused directly or through a linker to a second C_{H}1 region, which in turn is fused directly or through a linker to a second multimerizing component M2; a third polypeptide consisting essentially of a light chain variable domain (V_{L}) that is cognate with V_{H}1, and that is associated with a light chain constant region (C_{L}); and a fourth polypeptide that consists essentially of a light chain variable domain (V_{L}) that is cognate with VH3 and that is associated with a light chain variable domain. See, e.g., FIG. 7C.

In one embodiment, the V_{H}1 and V_{H}3 are derived from a non-human animal that expresses a single rearranged light chain variable domain from a single rearranged V/J light chain gene in the germline of the non-human animal, and the V_{L} that is cognate with V_{H}1 and the V_{L} that is cognate with V_{H}3 are derived from the same rearranged V/J light chain. In one embodiment, the V_{L}2sd is derived from a non-human animal that comprises a limited heavy chain repertoire (e.g., a repertoire having only a single heavy chain V segment and/or D and/or J segment, or a single rearranged heavy chain (V/D/J) gene. In one embodiment, the V_{H}1/V_{L}, V_{H}3/V_{L}, and V_{L}2sd each bind a different antigen. In one embodiment, at least two of the V_{H}1/V_{L}, V_{H}3/V_{L} and V_{L}2sd bind a different epitope of the same antigen, and the third binds a different antigen. See, e.g., FIG. 7C.

In one aspect, a multivalent antigen-binding protein is provided, comprising four polypeptides, wherein the first polypeptide comprises (from N-terminal to C-terminal) a first immunoglobulin heavy chain variable domain fused directly or through a linker to a first C_{H}1, which is fused directly or through a linker to a first multimerizing component M1, which is in turn fused directly or through a linker to a light chain single immunoglobulin variable domain (V_{L}2sd); a second polypeptide that comprises (from N-terminal to C-terminal) an immunoglobulin heavy chain variable domain (V_{H}3) fused directly or through a linker to a second C_{H}1, which in turn is fused directly or through a linker to a second multimerizing component M2, which in turn is fused directly or through a linker to an immunoglobulin single light chain variable domain (V_{L}3sd); a third polypeptide that comprises a light chain variable domain (V_{L}1) that is cognate with V_{H}1 and is associated with a light chain constant region; and a fourth polypeptide that comprises a light chain variable domain (V_{L}1) that is cognate with V_{H}3 and is associated with a light chain constant region. See, e.g., FIG. 7D.

In one embodiment, the multivalent antigen-binding protein consists essentially of four polypeptides, the first polypeptide consisting essentially of (from N-terminal to C-terminal) a first immunoglobulin heavy chain variable domain (V_{H}1) associated with a first C_{H}1, which is fused directly or through a linker to a first multimerizing component M1, which is in turn fused to a light chain immunoglobulin single variable domain (V_{L}2sd) that binds its target in the absence of a cognate heavy chain; a second polypeptide consisting essentially of (from N-terminal to C-terminal) an immunoglobulin heavy chain variable domain (V_{H}3) associated with a second C_{H}1 that is fused directly or through a linker to a second multimerizing component M2, which in turn is fused to a light chain immunoglobulin single variable domain (V_{L}3sd) that binds its target in the absence of a cognate heavy chain; a third polypeptide that consists essentially of a light chain immunoglobulin variable domain (V_{L}1) that is cognate with V_{H}1 and is associated with a light chain constant region (C_{L}); and a fourth polypeptide that consists essentially of a light chain immunoglobulin variable domain that is cognate with V_{H}3 and is associated with a light chain constant region. See, e.g., FIG. 7D.

In one embodiment, the V_{H}1 and V_{H}3 are derived from a non-human animal that expresses a single rearranged light chain from a single (V/J) rearranged light chain gene in the germline of the non-human animal, and the V_{L} that is cognate with V_{H}1 and the V_{L} that is cognate with V_{H}3 are the same rearranged light chain. In one embodiment, one or both of the light chain immunoglobulin single variable domains (V_{H}2sd, V_{L}3sd) are derived from a non-human animal that has a restricted heavy chain repertoire, e.g., a non-human animal that expresses immunoglobulin heavy chains that are derived from no more than a single heavy chain V gene segment, or no more than a single rearranged V/D/J gene.

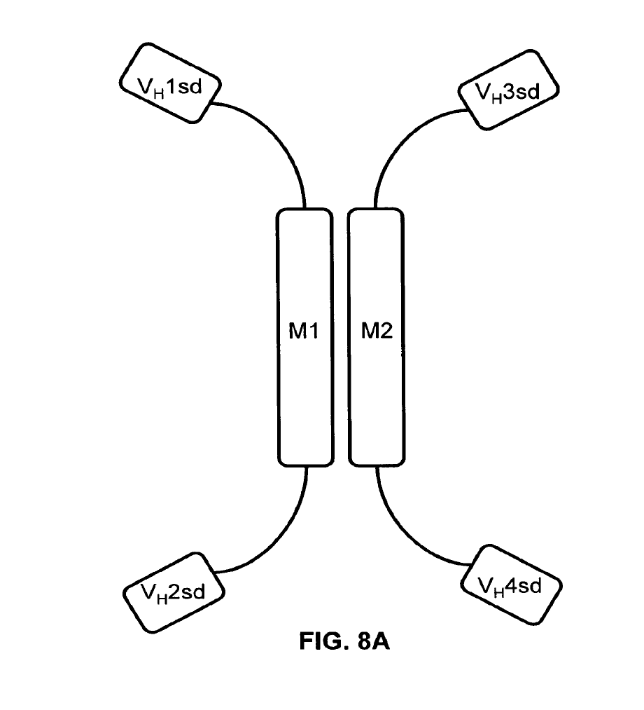

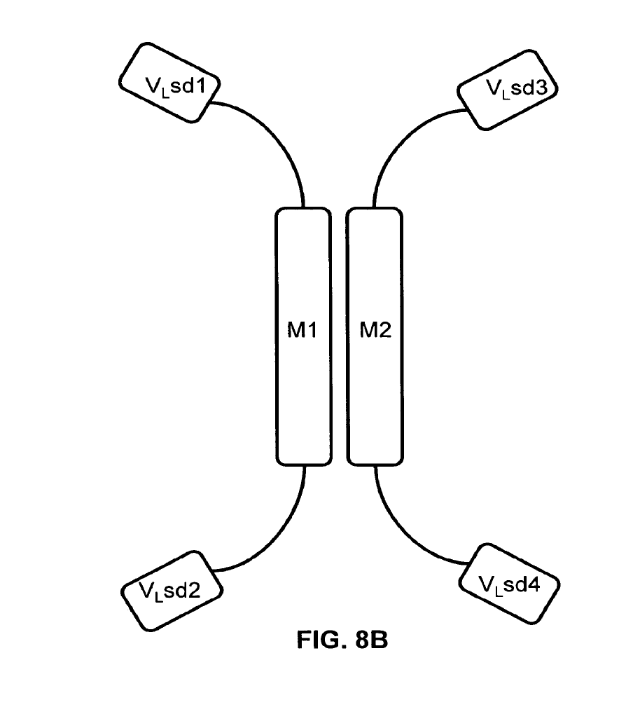

In one aspect, a multivalent antigen-binding protein is provided, comprising a first polypeptide that comprises a first immunoglobulin heavy chain variable domain (V_{H}1), optionally a linker, a first multimerizing component (M1), optionally a linker, and a second immunoglobulin heavy chain variable domain; and a second polypeptide that comprises (or consists essentially of) a third immunoglobulin heavy chain variable domain (V_{H}3), optionally a linker, a second multimerizing component (M2), optionally a linker, and a fourth heavy chain variable domain (V_{H}4); wherein the antigen-binding protein lacks a C_{H}1, and lacks an immunoglobulin light chain variable domain (see, e.g., FIG. 8A).

In one embodiment, the multivalent antigen-binding protein consists essentially of a first polypeptide and a second polypeptide. The first polypeptide consists essentially of a first heavy chain immunoglobulin single variable domain (V_{H}1sd) fused directly or through a linker to a first multimerizing component M1, which in turn is fused directly or through a linker to a second heavy chain immunoglobulin single variable domain (V_{H}2sd); and a second polypeptide that consists essentially of a third heavy chain immunoglobulin single variable domain (V_{H}3sd) fused directly or through a linker to a second multimerizing component M2, which in turn is fused directly or through a linker to a fourth heavy chain immunoglobulin single variable domain (VH4sd). See, e.g., FIG. 8A.

In various embodiments, the heavy chain immunoglobulin single variable domains (which do not require a cognate light chain domain to bind target) are derived from non-human animals that lack a C_{H}1 gene sequence or that lack a hinge gene sequence and lack a C_{H}1 gene sequence in an IgG.

In one aspect, a multivalent antigen-binding protein is provided that comprises a first polypeptide and a second polypeptide, wherein the first polypeptide comprises (from N-terminal to C-terminal) a first light chain immunoglobulin single variable domain (V_{L}sd1) fused directly or through a linker to a first multimerizing component M1, which in turn is fused directly or through a linker to a second light chain immunoglobulin single variable domain (V_{L}sd2); and the second polypeptide comprises (from N-terminal to C-terminal) a third light chain immunoglobulin single variable domain (V_{L}sd3) fused directly or through a linker to a second multimerizing component M2, which in turn is fused directly or through a linker to a fourth light chain immunoglobulin single variable domain (V_{L}sd4). In one embodiment, V_{L}sd1, V_{L}sd2, V_{L}sd3, V_{L}sd4 each bind a different epitope; in one embodiment, V_{L}sd1, V_{L}sd2, V_{L}sd3, and V_{L}sd4 each bind a different antigen; in one embodiment, V_{L}sd1, V_{L}sd2, V_{L}sd3, and V_{L}sd4 bind no more than three antigens (e.g., at least two of V_{L}sd1, VLsd2, V_{L}sd3, or V_{L}sd4 bind the same or a different epitope on a first same antigen, and the remaining two each bind a separate antigen that is not the first same antigen). See, e.g., FIG. 8B.

In one embodiment, the multivalent antigen-binding protein consists essentially of a first polypeptide and a second polypeptide, wherein the first polypeptide consists essentially of (from N-terminal to C-terminal) a first light chain immunoglobulin single variable domain (V_{L}sd1) fused directly or through a linker to a first multimerizing component M1, fused directly or through a linker to a second light chain immunoglobulin single variable domain (V_{L}sd2); and a second polypeptide consisting essentially of a third light chain immunoglobulin single variable domain (V_{L}sd3) fused directly or through a linker to a second multimerizing component M2, which is fused directly or through a linker to a fourth light chain immunoglobulin single variable domain (V_{L}sd4). See, e.g., FIG. 8B.

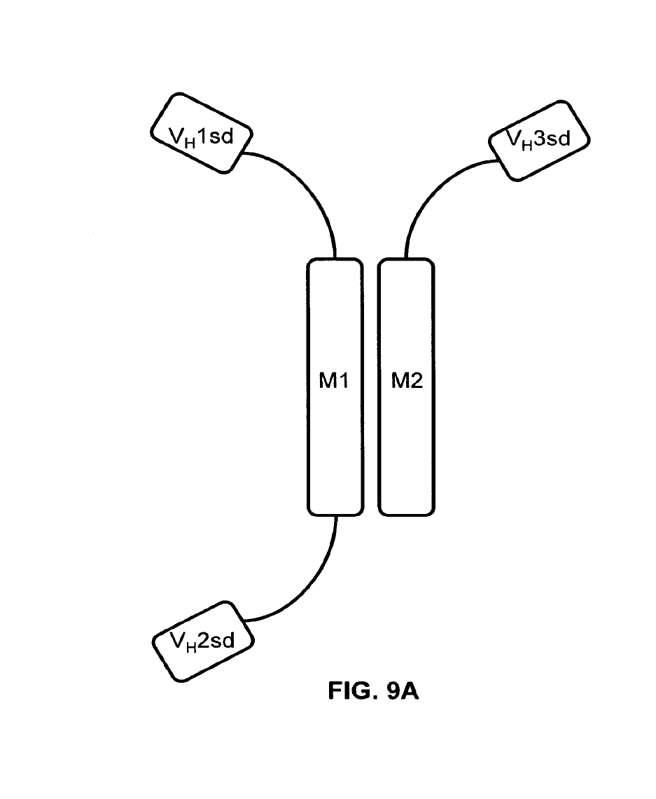

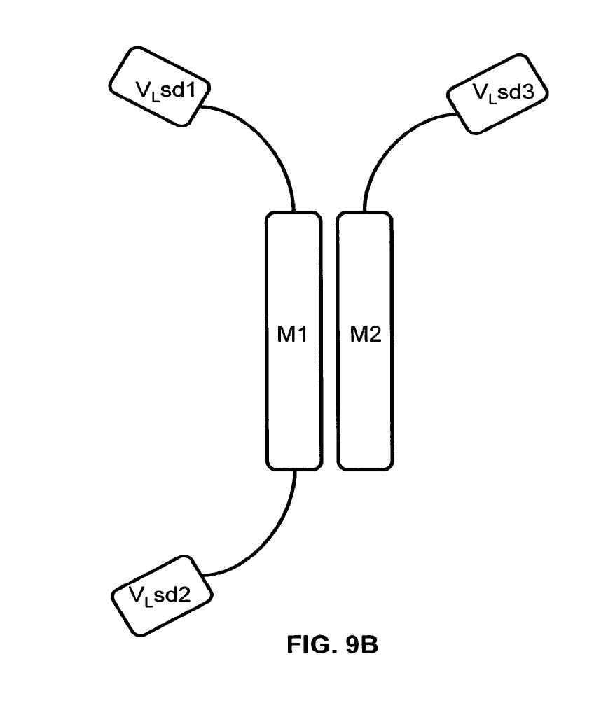

In one aspect, a multivalent antigen-binding protein is provided, comprising a first polypeptide that comprises a first immunoglobulin heavy chain variable domain (V_{H}1), optionally a linker, a first multimerizing component (M1), optionally a linker, and a second immunoglobulin heavy chain variable domain; and a second polypeptide that comprises (or consists essentially of) a third immunoglobulin heavy chain variable domain (V_{H}3), optionally a linker, a second multimerizing component (M2); wherein the antigen-binding protein lacks a C_{H}1, and lacks an immunoglobulin light chain variable domain (see, e.g., FIG. 9A).

In one embodiment, the multivalent antigen-biding protein consists essentially of a first polypeptide and a second polypeptide, wherein the first polypeptide consists essentially of (from N-terminal to C-terminal) a first heavy chain immunoglobulin single variable domain (V_{H}1sd) fused directly or through a linker to a first multimerizing component M1, which is fused directly or through a linker to a second heavy chain immunoglobulin single variable domain; and a second polypeptide consisting essentially of (from N-terminal to C-terminal) a third heavy chain immunoglobulin single variable domain fused directly or through a linker to a second multimerizing component M2. See, e.g., FIG. 9A.

In one aspect, a multivalent antigen-binding protein is provided, comprising a first polypeptide and a second polypeptide, wherein the first polypeptide comprises (from N-terminal to C-terminal) a first light chain immunoglobulin single variable domain (V_{L}sd1) fused directly or through a linker to a first multimerizing component M1, which is fused directly or through a linker to a second light chain immunoglobulin single variable domain (V_{L}sd2); wherein the second polypeptide comprises (from N-terminal to C-terminal) a third light chain immunoglobulin single variable domain (V_{L}sd3) fused directly or through a linker to a second multimerizing component M2. In one embodiment, V_{L}sd1, V_{L}sd2, and V_{L}sd3 bind three separate antigens; in one embodiment, V_{L}sd1, V_{L}sd2, and V_{L}sd3 bind three separate epitopes; in one embodiment, V_{L}sd1, V_{L}sd2, and V_{L}sd3 bind three epitopes on two antigens (i.e., one antigen contains two epitopes, and two of V_{L}sd1, V_{L}sd2, and V_{L}sd3 bind the two epitopes of the one antigen). See, e.g., FIG. 9B.

In one embodiment, the multivalent antigen-binding protein consists essentially of a first polypeptide and a second polypeptide, wherein the first polypeptide consists essentially of a first light chain immunoglobulin single variable domain (V_{L}sd1) fused directly or through a linker to the first multimerizing component M1, which is fused directly or through a linker to a second light chain immunoglobulin single variable domain (V_{L}sd2); and the second polypeptide consists essentially of a third light chain immunoglobulin variable domain that is fused directly or through a linker to the second multimerizing component M2.

In one embodiment, one or more of V_{L}sd1, V_{L}sd2, and V_{L}sd3 are derived from a non-human animal that comprises a limited heavy chain repertoire, e.g., that comprises a heavy chain repertoire that expresses only a single heavy chain and a plurality of light chains, such that binding specificity of an antibody or antigen-binding protein made in such a non-human animal resides primarily in the light chain variable domain. In one embodiment, V_{L}sd1, V_{L}sd2, and V_{L}sd3 are each derived from a non-human animal that expresses no more than one, or no more than two, heavy chain variable domains.

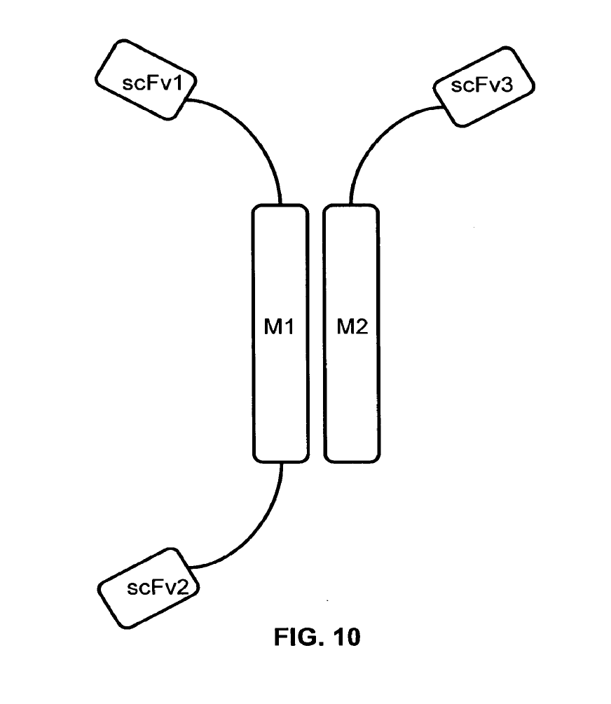

In one aspect, a multi-specific antigen-binding protein is provided, comprising a first polypeptide comprising a first scFv, optionally a linker, a first multimerizing component (M1), optionally a linker, and a second scFv; a second polypeptide that consists essentially of a third scFv, optionally a linker, and a second multimerizing component (M2) (see, e.g., FIG. 10). In one embodiment, the multivalent antigen-binding protein consists essentially of a first polypeptide and a second polypeptide, wherein the first polypeptide consists essentially of a first scFv (scFv1) fused directly or through a linker to the first multimerizing component M1, which is fused directly or through a linker to a second scFv; (scFv2) and a second polypeptide consisting essentially of a third scFv (scFv3) fused directly or through a linker to the second multimerizing component M2. In one embodiment, scFv1, scFv2, and scFv3 each bind a different antigen; in one embodiment two of scFv1, scFv2, and scFv3 bind the same first antigen (at, e.g., a different epitope), and the remaining scFv binds a second, different antigen.

In one aspect, a multivalent antigen-binding protein herein that lacks an immunoglobulin light chain variable domain comprises one or more immunoglobulin heavy chain variable domains whose sequence was obtained from a mouse that lacks an IgG gene that comprises a nucleotide sequence that encodes an IgG C_{H}1 domain. In one embodiment, the mouse comprises an IgM gene sequence that encodes an IgM C_{H}1 domain.

In one embodiment, the immunoglobulin heavy chain variable domain of a multivalent antigen-binding protein is derived from a human immunoglobulin heavy chain V gene segment selected from 1-72, 1-69, 1-58, 1-50, 1-42, 1-26, 1-18, 1-8, 3-6, 5-6, 7-1, 14-2, 14-2, and 14-1.

In one aspect, a multivalent antigen-binding protein as described herein is provided that comprises two or more different human immunoglobulin heavy chain variable domains that are associated with the same human immunoglobulin light chain variable domain, wherein the two or more different human immunoglobulin heavy chain variable domains are derived from a human heavy chain V gene segment selected from 1-18, 1-69, 2-5, 2-70, 3-9, 3-11, 3-13, 3-15, 3-20, 3-23, 3-30, 3-33, 3-48, 3-53, 3-64, 4-31, 4-34, 4-39, 4-59, 5-51, and 6-1.

In one embodiment, the same human immunoglobulin heavy chain variable domain is derived from a human Vκ1-39 gene segment or a human Vκ3-20 gene segment.

In one embodiment, the two or more human heavy chain V gene segments are selected from 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1; and the human immunoglobulin light chain variable domain is derived from a human Vκ1-39 gene segment.

In one embodiment, the two or more human heavy chain V gene segments are selected from 1-18, 1-69, 2-70, 3-11, 3-30, 3-33, 3-53, 4-39, 4-59, 5-51, 6-1; and the human immunoglobulin light chain variable domain is derived from a human Vκ3-20 gene segment.

In one embodiment, the light chain variable domain is encoded by a rearrangement of a germline human Vκ1-39 gene segment and a human J gene segment.

In one embodiment, the light chain variable domain is encoded by a rearrangement of a germline human Vλ3-20 gene segment and a human J gene segment.

In one embodiment, the light chain variable domain is encoded by a rearrangement of a germline human Vλ1-40 gene segment and a human J gene segment.

In one embodiment, the light chain variable domain is encoded by a rearrangement of a germline human Vλ2-14 gene segment and a human J gene segment.

In various specific embodiments, the human J gene segment is a Jκ or a Jλ gene segment.

In the various aspects described herein, the immunoglobulin variable domains, e.g., the light chain variable domains, the light chain single variable domains, the heavy chain variable domains, the heavy chain single variable domains, etc. are derived from rearranged variable gene sequences in humanized mice, e.g., humanized VELOCIMMUNE® humanized rodents, which comprise humanized rearranged immunoglobulin genes (e.g., a rearranged light chain gene, or a rearranged heavy chain gene) in their germline; or VELOCIMMUNE® humanized rodents that comprise unrearranged human V, D, and J (at an endogenous mouse heavy chain variable locus) and unrearranged human V and J (at an endogenous mouse light chain variable locus), as the case may be. For example, in various embodiments light chain immunoglobulin single variable domains can be made, e.g., in humanized rodents that comprise a single human rearranged heavy chain gene (or a single set of unrearranged V, D, and J heavy chain gene segments), and a full complement of human immunoglobulin light chain V and J gene segments, such that upon immunization such rodents will generate antibodies whose specificity resides largely in the light chain variable domain, and the antibodies (or B cells) may be screened in order to use the human light chain variable domains to bind target epitopes in the absence of a cognate heavy chain variable domain.

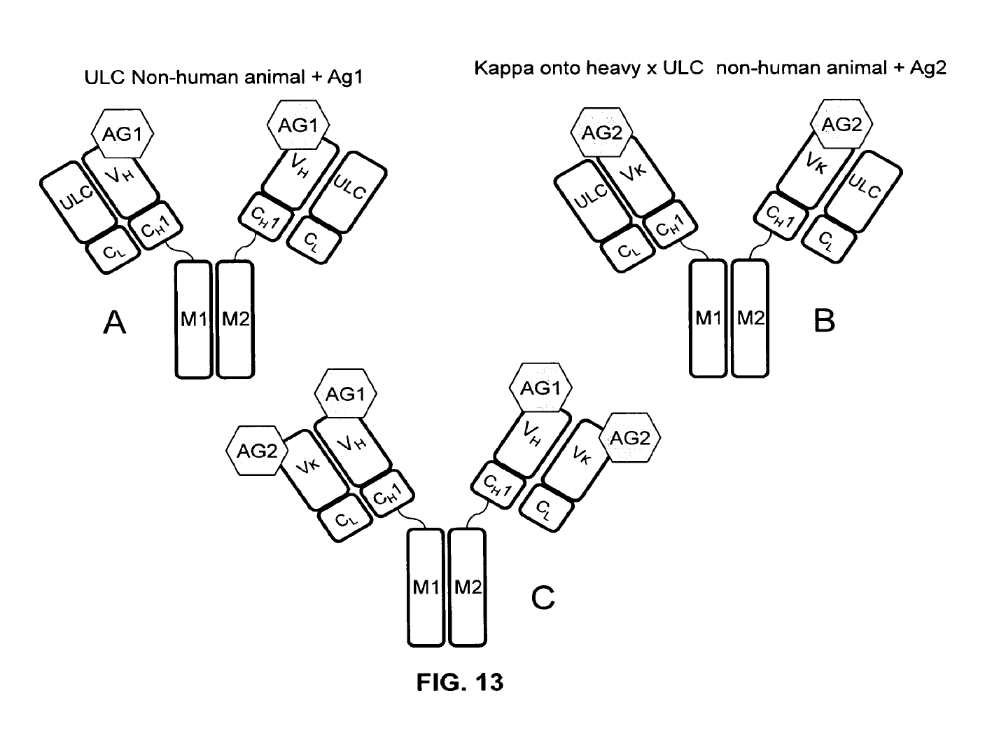

In one aspect, a multivalent antigen-binding protein is provided that binds a first antigen (AG1) through a first immunoglobulin heavy chain variable domain (V_{H}), wherein AG1 does not bind a light chain variable domain of the antigen-binding protein; and that binds a second antigen (AG2) through a first immunoglobulin light chain variable domain, wherein AG2 does not bind V_{H}. In one embodiment, the multivalent antigen-binding protein consists essentially of four polypeptides, wherein the first polypeptide consists essentially of (from N-terminal to C-terminal) a human heavy chain single variable domain that binds AG1, fused directly or through a linker to a C_{H}1 sequence, which is fused directly or through a linker to a first multimerizing component M1; the second polypeptide is identical to the first polypeptide, except that the C_{H}1 region is fused directly or through a linker to a second multimerizing component M2; and two copies of a third polypeptide, wherein the third polypeptide consists essentially of a human immunoglobulin Vκ single variable domain that binds AG2, wherein AG2 does not bind V_{H}, and wherein the Vκ single variable domain is fused directly or through a linker to a C_{L}. See, e.g., Panel C of FIG. 13. In one embodiment, the antigen-binding protein binds AG1 employing a first polypeptide (i.e., a first arm of the dimeric protein), and simultaneously binds AG2 employing the second polypeptide (i.e., a second arm of the dimeric protein).

In one embodiment, the multivalent antigen-binding protein variable domains are derived from (a) a non-human animal that comprises in its germline a humanized unrearranged heavy chain variable locus and expresses a single rearranged human light chain variable domain derived from a light chain variable locus that has a single human immunoglobulin light chain V gene segment (e.g., a universal light chain, or ULC, mouse); and (b) a non-human animal that comprises in its germline unrearranged human Vκ and Jκ gene segments operably linked to a heavy chain locus bred with a ULC mouse (e.g., a “human κ onto heavy×ULC” mouse). In one embodiment, AG1 is employed as an immunogen to immunize the ULC mouse of (a), and AG2 is employed as an immunogen to immunize the “human κ onto heavy×ULC” mouse of (b). The mouse of (a) immunized with AG1 are screened for antibodies or B cells that specifically bind AG1, and for heavy chains variable domains that bind AG1 without ULC binding to AG1, and such heavy chain sequences are cloned out of the mouse for use as V_{H} binding domains in the multivalent antigen-binding proteins. The mouse of (b) is immunized with AG2 and is screened for antibodies or B cells that specifically bind AG2, and for Vκ domains that bind AG2 in the absence of the ULC variable domain, and sequences encoding such Vκ domains are cloned out for use as Vκ domains in the multivalent antigen-binding proteins. In one embodiment, the V_{H} is cloned onto a C_{H}1, a hinge, a C_{H}2, and a C_{H}3 of a desired Ig (with, e.g., any desired further modifications of the hinge, C_{H}2, and/or C_{H}3). In one embodiment, the Vκ is cloned onto a light chain constant region, e.g., a human Cκ. For illustration purposes, the antigen-binding proteins that can be made at each stage to arrive at the multivalent antigen-binding protein that independently binds AG1 and AG2 are depicted in FIG. 13, Panels A and B, and an embodiment of the multivalent antigen-binding protein that independently binds both AG1 and AG2 is depicted in Panel C.

Suitable ULC non-human animals include mice that comprise a replacement at the endogenous mouse heavy chain locus of all or substantially all mouse V, D, and J gene segments with all or substantially all functional human V, D, and J gene segments, wherein the human gene segments are operably linked to a mouse heavy chain constant gene; and a mouse light chain locus that comprises a replacement of all functional mouse light chain V and J sequences with a single rearranged human κ V/J rearranged gene operably linked to a non-human (e.g., mouse or rat) light chain constant gene, e.g., a mouse or rat Cκ constant gene. Suitable universal light chain mice are described in, e.g., US Patent Application Publication Nos. 2011/0195454, 2012/0621409, and 2012/0192300 (each hereby incorporated by reference); suitable mice that comprise human Vκ and Jκ segments operably linked to non-human (e.g., mouse) heavy chain constant region genes (i.e., “κ onto heavy mice”) are disclosed in US Patent Application Publication No. 2012/0096572 (hereby incorporated by reference). Breeding of a ULC mouse and a “κ onto heavy mouse” will produce a mouse suitable for generating human Vκ variable domains that bind antigen in the presence of a universal light chain that neither interferes with binding of antigen by the human Vκ variable domain nor requires the participation of universal light chain CDR sequences to bind the antigen.

### Multivalent Binding Proteins: Immunoglobulin Variable Domain Elements

In various aspects, antigen-binding proteins are provided that comprise heavy chain variable domains (or functional fragments thereof) associated with an immunoglobulin light chain sequence. For example, see FIGS. 1A and 2A.

In various embodiments, the light chain sequences are derived from light chain elements, e.g., light chain variable domains, which can associate (e.g., can express with) two or three or more different heavy chain variable domains (or functional fragments thereof). A variety of methods are known in the art for generating light chains that can pair with two heavy chains of differing specificity, while not interfering or not substantially interfering with the selectivity and/or affinity of the heavy chain variable domain with its target antigen.

In one aspect, a light chain is selected by surveying usage statistics for all light chain variable domains, identifying the most frequently employed light chain in human antibodies, and pairing that light chain with the two heavy chains of differing specificity. In one aspect, a light chain can be selected by observing light chain sequences in a phage display library (e.g., a phage display library comprising human light chain variable region sequences, e.g., a human scFv library) and selecting the most commonly used light chain variable region from the library. In one aspect, a light chain can be selected by assaying a phage display library of light chain variable sequences using the heavy chain variable sequences of both heavy chains as probes. A light chain that associates with both heavy chain variable sequences is selected as a light chain for the heavy chains and allows binding and/or activation with respect to both epitopes. In one aspect, a light chain can be selected by combining known light chains with desired heavy chains and assaying the resulting multivalent antigen-binding protein for binding specificity, affinity, and/or blocking or activation ability or some other functional consequence of binding.

In one aspect, to the extent that a difficulty is encountered in any of the approaches for selecting a light chain (e.g., the light chain interferes with the binding of one or both of the heavy chains with its antigen, or the light chain fails to associate satisfactorily with one or both of the heavy chains), the light chain can be aligned with cognate light chains of the heavy chain variable domains, and modifications are made in the light chain to more closely match sequence characteristics common to the cognate light chains of the multiple heavy chains. If the likelihood of immunogenicity must be minimized, the modifications preferably result in sequences that are present in known human light chain sequences, such that proteolytic processing is unlikely to generate a T cell epitope based on parameters and methods known in the art for assessing the likelihood of immunogenicity (i.e., in silico as well as wet assays).

In one aspect, a suitable light chain variable domain is a universal light chain disclosed, e.g., in US Patent Application Publication Nos. 2012/0192300, 2012/021409, 2011/0195454, and U.S. Ser. No. 13/488,628 filed 5 Jun. 2012. In various embodiments, the light chain variable domain is derived from a germline Vκ segment selected from a Vλ1-39 segment and a Vλ3-20 segment. In a specific embodiment, the human V_{L} gene segment is a human Vλ1-39Jκ5 gene segment or a human Vλ3-20Jκ1 gene segment.

In one embodiment, the light chain is derived from a human Vλ1-39/Jκ (e.g., any Jκ, e.g., a Jκ5) rearrangement or a human Vλ3-20Jκ (e.g., any Jκ, e.g., a Jκ1) rearrangement, and the light chain has at least one or no more than four somatic hypermutations. In one embodiment, the light chain comprises at least two somatic hypermutations. In one embodiment, the light chain comprises at least three somatic hypermutations. In one embodiment, the light chain comprises at least four somatic hypermutations. In a specific embodiment, the mutations are present in one or more framework regions of the light chain. In a specific embodiment, the mutations are present in one or more CDR regions of the light chain. In a specific embodiment, the mutations are present in one or more framework regions and/or one or more CDR regions of the light chain. In various embodiments, the framework regions are selected from framework 1 (FR1), framework 2 (FR2), framework 3 (FR3), and/or a combination thereof.

In various aspects, multivalent antigen-binding proteins are provided that comprise two or more (different) heavy chain variable domains (or functional fragments thereof) that are each associated with an immunoglobulin light chain sequence that is derived from a single rearranged light chain variable domain derived from a non-human animal that expresses light chains derived from a single light chain V gene segment. Non-human animals comprising an unrearranged humanized heavy chain variable locus and a light chain variable locus that is humanized and is capable of rearranging light chain variable genes derived from a light chain V repertoire consisting of a single light chain V gene segment are described in US Patent Application Publications 2011/0195454A1, 2012/0021409A1, and 2012/0192300A1 (each publication hereby incorporated by reference).

In various embodiments, heavy chain variable domains that are cognate with light chains derived from the same light chain V gene segment (e.g., with the same V/J rearranged light chain) are suitable for use with various embodiments of the invention, e.g., as V_{H}1, V_{H}2, V_{H}3, and V_{H}4 in FIG. 1A and FIG. 1B, FIG. 2A, FIG. 2B, FIG. 2C, FIG. 2D, FIG. 6, FIG. 7A, FIG. 7B, FIG. 7C, and FIG. 7D.

In one aspect, a multivalent antigen-binding protein according to FIG. 1A is provided, wherein V_{L} is a human light chain variable domain derived from Vκ1-39/J rearrangement, C_{L} is a human Cκ, and V_{H}1 and V_{H}2 are human heavy chain variable domains derived from a non-human animal that comprises a light chain repertoire restricted to a human Vκ1-39/J rearrangement. In one embodiment, V_{H}1 and V_{H}2 are different. In one embodiment, V_{H}1 and V_{H}2 specifically bind two different epitopes on the same antigen or different antigens. In a specific embodiment, V_{H}1 and/or V_{H}2 are derived from a heavy chain gene segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, the heavy chain variable domain cognate with the Vκ1-39/J variable domain is rearranged with a D gene segment selected from a D1-1, 1-7, 2-8, 3-3, 3-10, 3-16, 3-22, 5-5, 5-12, 6-6, 6-13, and 7-27. In one embodiment, the heavy chain variable domain cognate with the Vκ1-39/J variable domain is a rearrangement of an above-mentioned V_{H} gene segment, an above-mentioned D gene segment, and a J_{H} gene segment selected from J1, 2, 3, 4, 5, and 6. In one embodiment, the V_{H}3 and the V_{H}4 are also rearrangements of the above-mentioned V, D, and J gene segments, and the cognate VL is the human light chain variable domain derived from Vκ1-39/J as above.

In one aspect, the multivalent antigen-binding protein according to FIG. 1A is provided wherein the V_{L} associated with V_{H}3 and V_{H}4 is a human light chain variable domain derived from a Vκ3-20/J rearrangement, C_{L} is a human Cκ, and V_{H}3 and V_{H}4 are human heavy chain variable domains derived from a non-human animal that comprises a light chain repertoire restricted to a human Vκ3-20/J rearrangement. In one embodiment, V_{H}3 and V_{H}4 are different. In one embodiment, V_{H}3 and V_{H}4 specifically bind two different epitopes on the same antigen or on different antigens. In a specific embodiment, V_{H}2 and/or V_{H}3 are derived from a heavy chain gene segment selected from a V_{H} 1-18, 1-69, 2-70, 3-11, 3-30, 3-33, 3-53, 4-39, 4-59, 5-51, and 6-1. In one embodiment, the heavy chain variable domain cognate with the Vκ3-20/J variable domain is a rearrangement of an above-mentioned V_{H} gene segment and a D gene segment selected from D 1-1, 1-7, 1-26, 2-15, 3-3, 3-16, and 6-13. In one embodiment, the heavy chain variable domain cognate with the Vκ3-20/J is a rearrangement of an above-mentioned V_{H} gene segment, an above-mentioned D gene segment, and a J_{H} segment selected from a J2, 3, 4, 5, and 6. In one embodiment, the V_{H}1 and the V_{H}2 are also rearrangements of the above-mentioned (this paragraph) V, D, and J gene segments, and the cognate V_{L} is the human light chain variable domain derived from Vκ3-20/J.

In one aspect, a multivalent antigen-binding protein according to FIG. 1B is provided, wherein V_{H}1 and V_{H}3 (or V_{H}2 and V_{H}4) are derived from a humanized mouse that comprises a humanized heavy chain variable repertoire and a restricted light chain repertoire characterized by being derived from a single human immunoglobulin light chain V gene segment and a J segment, wherein V_{L}1 (or V_{L}2) is derived from the same single human light chain V gene segment and J segment. In one embodiment, the light chain V gene segment is a Vκ1-39 gene segment. In one embodiment, where the light chain V gene segment is a Vκ1-39 gene segment, the V_{H}1 and V_{H}3 are each independently derived from a V_{H} gene segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, the V_{H}1 and V_{H}3 (or V_{H}2 and V_{H}4) are derived from a humanized mouse that comprises a restricted light chain repertoire characterized by being derived from a single human immunoglobulin light chain V gene segment and a J segment, wherein V_{L}1 (or V_{L}2) is derived from the same single human light chain V gene segment and J segment. In one embodiment, the light chain V gene segment is a Vκ3-20 gene segment. In one embodiment, where the light chain V gene segment is a Vλ3-20 gene segment, the V_{H}1 and V_{H}3 are each independently derived from a V_{H} gene segment selected from a V_{H} 1-18, 1-69, 2-70, 3-11, 3-30, 3-33, 3-53, 4-39, 4-59, 5-51, and 6-1.

In one aspect, a multivalent antigen-binding protein according to FIG. 2A is provided, wherein each of V_{H}1, V_{H}2, and V_{H}3 are derived from a humanized mouse comprising a humanized heavy chain variable repertoire and a restricted light chain repertoire characterized by being derived from a single human immunoglobulin light chain V gene segment and a J segment, wherein V_{L} is derived from the same single human light chain V gene segment and J segment. In one embodiment, the light chain V gene segment is a Vκ1-39 gene segment, and V_{H}1, V_{H}2, and V_{H}3 are independently derived from a V_{H} gene segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, the light chain V gene segment is a Vλ3-20 gene segment, and the V_{H}1, V_{H}2, and V_{H}3 are independently derived from a V_{H} gene segment selected from a V_{H} 1-18, 1-69, 2-70, 3-11, 3-30, 3-33, 3-53, 4-39, 4-59, 5-51, and 6-1.

In one aspect, a multivalent antigen-binding protein according to FIG. 2B or FIG. 2C is provided, wherein the V_{H}2 and V_{H}4 are derived from a humanized mouse comprising a humanized heavy chain variable repertoire and a restricted light chain repertoire characterized by being derived from a single human immunoglobulin light chain V gene segment and a J segment, wherein V_{L} (or, in FIG. 2C, V_{L}2) is derived from the same single human light chain V gene segment and J segment. In one embodiment, the light chain V gene segment is a Vκ1-39 gene segment, and V_{H}2 and V_{H}4 are independently derived from a V_{H} gene segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, the light chain V gene segment is a Vκ3-20 gene segment, and the V_{H}2 and V_{H}4 are independently derived from a V_{H} gene segment selected from a V_{H} 1-18, 1-69, 2-70, 3-11, 3-30, 3-33, 3-53, 4-39, 4-59, 5-51, and 6-1.

In one aspect, a multivalent antigen-binding protein according to FIG. 2D is provided, wherein the V_{H}1 is derived from a first humanized mouse comprising a humanized heavy chain variable repertoire and a restricted human light chain repertoire derived from a first single human V gene segment and a J segment, and V_{H}2 is derived from a second humanized mouse comprising a humanized heavy chain variable repertoire and a restricted human light chain repertoire derived from a second human light chain V gene segment and a J segment, wherein the first single human light chain V gene segment and the second human light chain V gene segment are not the same. In one embodiment, the first and the second human V gene light chain gene segments are selected from a human Vκ1-39 gene segment and a human Vκ3-20 gene segment, and the C_{L} is a human Cκ. In one embodiment, for cognate heavy chain variable domains associated with a Vκ1-39-derived light chain, the heavy chain is derived from a human V_{H} segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, for cognate heavy chain variable domains associated with a Vκ3-20-derived light chain, the heavy chain is derived from a human V_{H} segment selected from a V_{H} 1-18, 1-69, 2-70, 3-11, 3-30, 3-33, 3-53, 4-39, 4-59, 5-51, and 6-1. In an embodiment where the light chain constant region is a human κ sequence, the C_{L} is a human Cκ.

In one aspect, a multivalent antigen-binding protein according to FIG. 6 is provided, V_{H}1, V_{H}2, V_{H}3, and V_{H}4 are derived from a non-human animal with a humanized unrearranged heavy chain variable region locus, wherein the non-human animal comprises a restricted humanized light chain variable repertoire characterized by expressing light chains derived from just a single human immunoglobulin light chain variable gene segment (in rearrangement with a human J segment). In one embodiment, the single human immunoglobulin light chain variable gene segment is selected from a human Vκ1-39 gene segment and a human Vκ3-20 gene segment. In one embodiment, the human light chain variable domain is derived from a human Vκ1-39/J gene segment, and the V_{H}1, V_{H}2, V_{H}3, and V_{H}4 are independently derived from a human V_{H} gene segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, the human light chain variable domain is derived from a human Vκ3-20/J gene segment, and the V_{H}1, V_{H}2, V_{H}3, and V_{H}4 are independently derived from a human V_{H} gene segment selected from a V_{H} 1-18, 1-69, 2-70, 3-11, 3-30, 3-33, 3-53, 4-39, 4-59, 5-51, and 6-1. In one embodiment, V_{H}1, V_{H}2, V_{H}3, and V_{H}4 are independently selected from a heavy chain variable segment that is cognate with a human light chain variable domain derived from a rearranged human Vκ1-39/J sequence and a human Vκ3-20 sequence. In one embodiment, the binding moieties V_{H}1/V_{L}, V_{H}2/V_{L}, V_{H}3/V_{L}, and V_{H}4/V_{L} each bind a different epitope on one, two, three, or four different antigens. In one embodiment, the binding moieties V_{H}1/V_{L}, V_{H}2/V_{L}, V_{H}3/V_{L}, and V_{H}4/V_{L} bind epitopes on two or more antigens, or three or more antigens. In one embodiment, where the V_{L} is derived from a human Vκ sequence, the CL is a Cκ.

In one aspect, a multivalent antigen-binding protein according to FIG. 7A is provided, wherein V_{H}1, V_{H}2, and V_{H}3 are derived from a non-human animal with a humanized unrearranged heavy chain variable region locus, wherein the non-human animal comprises a restricted humanized light chain variable repertoire characterized by expressing light chains derived from just a singe human immunoglobulin light chain variable domain (in rearrangement with a human J segment). In one embodiment, the single human immunoglobulin light chain variable gene segment is selected from a human Vκ1-39 gene segment and a human Vλ3-20 gene segment. In one embodiment, the human light chain variable domain is derived from a human Vκ1-39/J gene, and the V_{H}1, V_{H}2, and V_{H}3 are independently derived from a human V_{H} gene segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, the human light chain variable domain is derived from a human Vκ3-20/J gene, and the V_{H}1, V_{H}2, and V_{H}3 are independently derived from a human V_{H} gene segment selected from a V_{H} 1-18, 1-69, 2-70, 3-11, 3-30, 3-33, 3-53, 4-39, 4-59, 5-51, and 6-1. In one embodiment, V_{H}1, V_{H}2, and V_{H}3 are independently selected from a heavy chain variable segment that is cognate with a human light chain variable domain derived from a rearranged human Vκ1-39/J sequence and a human Vκ3-20 sequence. In one embodiment, the binding moieties V_{H}1/V_{L}, V_{H}2/V_{L}, and V_{H}3/V_{L}, each bind a different epitope on one, two, or three different antigens. In one embodiment, the binding moieties V_{H}1/V_{L}, V_{H}2/V_{L}, and V_{H}3/V_{L}, bind epitopes on two or more antigens. In one embodiment, where the V_{L} is derived from a human Vκ sequence, the C_{L} is a Cκ.

In one aspect, a multivalent antigen-binding protein is provided according to FIG. 7B, 7C, or 7D, wherein V_{H}1 and V_{H}3 are derived from a non-human animal comprising a humanized heavy chain variable locus, wherein the non-human animal comprises a restricted light chain repertoire characterized by expressing just one light chain derived from a single human light chain variable segment (rearranged with a human J segment). In one embodiment, the single human light chain variable segment is selected from a human Vκ-39 segment and a human Vκ3-20 segment. In one embodiment, the cognate V_{L} to V_{H}1 and/or V_{H}3 is independently selected from a VL derived from a Vκ1-39 gene segment and a Vκ3-20 gene segment. In one embodiment, when the V_{H}1 or V_{H}3 is cognate with a V_{L} derived from a Vκ1-39 segment, the V_{H}1 or V_{H}3 is derived from a V_{H} segment selected from a V_{H} 1-18, 1-69, 2-5, 3-9, 3-13, 3-15, 3-20, 3-23, 3-30, 3-48, 3-53, 3-64, 4-31, 4-34, 4-39, 4-59, 5-51, and 6-1. In one embodiment, when the V_{H}1 or V_{H}3 is cognate with a V_{L} derived from a Vκ3-20 segment, the V_{H}1 or V_{H}3 is derived from a V_{H} segment selected from a V_{H} 1-18, 1-69, 2-70, 3-11, 3-30, 3-33, 3-53, 4-39, 4-59, 5-51, and 6-1. In one embodiment, where the V_{L} is derived from a human Vκ sequence, the C_{L} is a Cκ.

In various aspects of the multivalent antigen-binding proteins of FIGS. 1A, 1B, 2A, 2B, 2C, 2D, 6, 7A, 7B, 7C, and 7D in which a human heavy chain variable domain is cognate with a human light chain variable domain from a non-human animal comprising a humanized heavy chain variable domain and a light chain variable repertoire limited to being derived from a single human V gene segment rearranged with a single human light chain J gene segment, the single human rearranged light chain V/J sequence is rearranged from germline sequences in the absence of N or P additions. Thus, the non-human animal comprises a light chain repertoire in its germline characterized by a single rearranged germline V/J sequence that lacks N or P additions. In various aspects and embodiments, such rearranged germline V/J sequences form cognate V_{H}/V_{L} pairs with a wide variety of V_{H} domains derived from a wide variety of human V gene segments. Thus, in one aspect, the multivalent antigen-binding domains of the indicated figures comprise V_{H} domains derived from the following human heavy chain V gene segments: V_{H}1-2, V_{H}1-3, V_{H}1-8, V_{H}1-18, V_{H}1-24, V_{H}1-45, V_{H}1-46, V_{H}1-58, V_{H}1-69, V_{H}2-5, V_{H}2-26, V_{H}2-70, V_{H}3-7, V_{H}3-9, V_{H}3-11, V_{H}3-13, V_{H}3-15, V_{H}3-16, V_{H}3-20, V_{H}3-21, V_{H}3-23, V_{H}3-30, V_{H}3-30-3, V_{H}3-30-5, V_{H}3-33, V_{H}3-35, V_{H}3-38, V_{H}3-43, V_{H}3-48, V_{H}3-49, V_{H}3-53, V_{H}3-64, V_{H}3-66, V_{H}3-72, V_{H}3-73, V_{H}3-74, V_{H}4-4, V_{H}4-28, V_{H}4-30-1, V_{H}4-30-2, V_{H}4-30-4, V_{H}4-31, V_{H}4-34, V_{H}4-39, V_{H}4-59, V_{H}4-61, V_{H}5-51, V_{H}6-1, V_{H}7-4-1 and V_{H}7-81, wherein the V_{H} domains are each independently cognate with at least one V_{L} domain that is derived from a rearranged germline V/J human immunoglobulin light chain sequence in the germline of the non-human animal. In various embodiments, the rearranged light chain V/J sequence is a human Vκ/Jκ sequence. In various embodiments, the rearranged light chain V/J sequence is a human Vλ/J?, sequence.

### Methods and Applications

The compositions and methods of described herein can be used to make binding proteins that bind more than one epitope with high affinity, low affinity, or mixed high affinity/low affinity (e.g., one or more V_{H} bind with low affinity, and one or more V_{H} bind with high affinity to the same or different epitope). Advantages of the invention include the ability to select suitable heavy chain immunoglobulin variable domains, each of which will associate with the same, or very nearly the same, light chain variable domain. The heavy chain variable domains can be selected to have any desirable combination of properties, e.g., high affinity to a first epitope (E1), moderate or low affinity to a second epitope (E2), or high affinity to a third epitope (E3) on the same or a different antigen (or cell, e.g.) as E1 and/or E2; and optionally a fourth epitope (E4). The heavy chain variable domains can be selected to simultaneously bind any two (or more) antigens or cell types, or, e.g., an antigen (e.g., a protein antigen) and one or more cells.

In various aspects, a multivalent antigen-binding protein is provided that has no more than three different heavy chain variable domains, wherein at least one variable domain specifically binds a target on an effector cell, e.g., a cell surface molecule of a T cell, e.g., a CD3. In one embodiment, the remaining two different heavy chain variable domains bind the same or a different antigen on a target cell, e.g., a tumor antigen (e.g., CD20).

Synthesis and expression of multivalent binding proteins has been problematic, in part due to issues associated with identifying a suitable light chain that can associate and express with two or more different heavy chains, and in part due to isolation issues. The methods and compositions described herein allow for making suitable multivalent binding proteins by suitable method. Suitable methods may include phage display methods (including modification of germline sequences generated in phage display systems), and other in vitro methods known in the art. A particularly useful method is having a genetically modified mouse make, through natural processes, a suitable heavy chain variable domain that can associate and express with a common or universal light chain.

In various aspects, human V_{H} sequences from suitable B cells of immunized rodents that are genetically engineered to express human light chain variable domains derived from no more than one, or no more than two, human V gene segments are used as a source of potential V_{H} domains for a multivalent antibody. The B cells are from rodents that are immunized with one or more antigens of interest, which are, in various embodiments, antigens to which the multivalent antibody will bind. Cells, tissues, or liquids of the rodents are screened to obtain heavy chain variable domains (or B cells that express them) that exhibit desired characteristics with respect to the antigens of interest, e.g., high affinity, low affinity, blocking ability, activation, or some other functional characteristic. Because all of the V_{H} domains are made in a mouse that expresses human immunoglobulin light chain derived from no more than one, or no more than two, V_{L} gene segments, all V_{H} domains are capable of expressing and associating with V_{L} domains that are expressed in the mouse.

Cells or tissues from rodents as described herein that express affinity matured antibodies having reverse chimeric heavy chains (i.e., human variable and mouse constant) can be used to generate heavy chain variable domains that are identified and cloned in frame in an expression vector with a suitable human constant region gene sequence (e.g., a human IgG1), useful in making multivalent antigen-binding proteins of the invention. Two such constructs can be prepared, wherein each construct encodes a human heavy chain variable domain that binds a different epitope (or the same epitope). One of the human VLs (e.g., human Vλ1-39Jκ5 or human Vλ3-20Jκ1), in germline sequence or from a B cell wherein the sequence has been somatically mutated, can be fused in frame to a suitable human constant region gene (e.g., a human κ constant gene). These three fully human heavy and light constructs can be placed in a suitable cell for expression. The cell will express two major species: a homodimeric heavy chain with the identical light chain, and a heterodimeric heavy chain with the identical light chain. To allow for a facile separation of these major species, one of the heavy chains is modified to omit a Protein A-binding determinant, resulting in a differential affinity of a homodimeric binding protein from a heterodimeric binding protein. Compositions and methods that address this issue are described in U.S. Ser. No. 12/832,838, filed 25 Jun. 2010, entitled “Readily Isolated Bispecific Antibodies with Native Immunoglobulin Format,” published as US 2010/0331527A1, hereby incorporated by reference.

In one aspect, an epitope-binding protein as described herein is provided, wherein human V_{L} and V_{H} sequences are derived from mice described herein that have been immunized with an antigen comprising an epitope of interest.

### Multimerizing Components

In one aspect, the multimerizing component M1 and M2 are the same. In one aspect, the multimerizing component M1 and M2 are different.

In one aspect, the multimerizing component is selected from a leucine zipper, a zinc finger, an immunoglobulin light chain constant domain, and an Fc domain. In one embodiment, the multimerizing component is an Fc of an IgG. In one embodiment, the immunoglobulin light chain constant domain is a Cκ or a Cκ. In one embodiment, the Cκ or Cλ is a human Cκ or Cλ.

In one embodiment, the Fc is from an IgG of isotype IgG1, IgG2, IgG3, and IgG4. In one embodiment, the multimerizing component comprises a sequence selected from a human IgG1, a human IgG2, a human IgG3, a human IgG4, and a combination thereof. In a specific embodiment, the multimerizing component contains a C_{H}2 and a C_{H}3 of a human IgG selected from IgG1, IgG2, IgG3, and IgG4. In one embodiment, the multimerizing component contains a C_{H}2 and a C_{H}3 of a human IgG1, IgG2, IgG3, or IgG4, and is modified as described herein.

In one aspect, M1 and M2 each independently comprise an immunoglobulin heavy chain constant domain or multimerizing fragment thereof (e.g., an Fc or a multimerizing fragment thereof). In one embodiment, the immunoglobulin heavy chain constant domain or multimerizing fragment thereof is human. In one embodiment, M1 and M2 each independently comprise an immunoglobulin heavy chain constant domain selected from C_{H}2, C_{H}3, and a combination thereof. In a specific embodiment, M1 and M2 each independently comprise a human C_{H}2 and C_{H}3, arranged, e.g., as found in a human Fc, e.g., in a human IgG1, IgG2, IgG3, or IgG4 Fc.

In various aspects and embodiments, M1 and M2 comprise immunoglobulin constant domains, or multimerizing portions thereof, that are differentially modified, i.e., modifications present in M1 are not present in M2, and modifications present in M2 are not present in M1. Unless otherwise specified, modifications that are recited in connection with M1 may be used with M2, and vice versa. That is, the modifications mentioned throughout for M1 may be used on M2 for any embodiment, and the modifications mentioned throughout for M2 may be used on M1 for any embodiment.

In one embodiment, the first polypeptide comprises a M1 that comprises an immunoglobulin heavy chain constant domain that comprises a first C_{H}3 region of a human IgG selected from IgG1, IgG2, IgG4, and a combination thereof; and the second polypeptide comprises a M2 that comprises an immunoglobulin constant region that comprises a second C_{H}3 region of a human IgG selected from IgG1, IgG2, IgG4, and a combination thereof, wherein the second C_{H}3 region comprises a modification that reduces or eliminates binding of the second C_{H}3 domain to protein A. In one embodiment, the first C_{H}3 region (but not the second C_{H}3 region) comprises a modification that reduces or eliminates binding of the first C_{H}3 domain to protein A.

In one embodiment, the second C_{H}3 region (or the first C_{H}3 region, but not both) comprises a 95R modification (by IMGT exon numbering; 435R by EU numbering). In another embodiment, the second C_{H}3 region (or the first C_{H}3 region, but not both) further comprises a 96F modification (IMGT; 436F by EU), i.e., a human IgG1ΔAdp modification (IgG1/95R/96F).

In one embodiment, the second C_{H}3 region (or the first C_{H}3, but not both) is from a modified human IgG1, and further comprises a modification selected from the group consisting of D16E, L18M, N44S, K52N, V57M, and V82I (IMGT; D356E, L358M, N384S, K392N, V397M, and V422I by EU).

In one embodiment, the second C_{H}3 region (or the first C_{H}3, but not both) is from a modified human IgG2, and further comprises a modification selected from the group consisting of N44S, K52N, and V82I (IMGT; N384S, K392N, and V422I by EU).

In one embodiment, the second C_{H}3 region (or the first C_{H}3 region, but not both) is from a modified human IgG4, and further comprises a modification selected from the group consisting of Q15R, N44S, K52N, V57M, R69K, E79Q, and V82I (IMGT; Q355R, N384S, K392N, V397M, R409K, E419Q, and V422I by EU). In one embodiment, the C_{H}3 domain of M1 or M2 is a chimeric domain that comprises sequences of two or more of human IgG1, human IgG2, human IgG3, and human IgG4. In one embodiment, the chimeric C_{H}3 domain of M1 is not identical to the chimeric C_{H}3 domain of M2.

In one embodiment, the C_{H}3 domain of M1 and/or M2 is from human IgG1, human IgG2, or human IgG4, and the polypeptide comprising M1 and the polypeptide comprising M2 each independently further comprises a C_{H}1 domain and a C_{H}2 domain, wherein the C_{H}1 domain and the C_{H}2 domain are independently selected from the group consisting of a human IgG1 C_{H}1 or C_{H}2 domain, a human IgG2 C_{H}1 or C_{H}2 domain, or a chimeric human/human IgG1/IgG2 or a chimeric human/human IgG1/IgG3 or a chimeric human/human IgG2/IgG3 domain or a chimeric human/human IgG1/IgG4 or a chimeric IgG3/IgG4 or a chimeric IgG2/IgG4 domain. In a specific embodiment, the chimeric IgG1/IgG2, IgG1/IgG3, IgG2/IgG3, IgG1/IgG4, IgG3/IgG4, and IgG2/IgG4 domains are non-immunogenic or substantially non-immunogenic in a human.

In one embodiment, the antigen-binding protein is non-immunogenic or substantially non-immunogenic in a human. In one embodiment, the antigen-binding protein lacks a non-native human T-cell epitope in a heavy chain constant domain; in a specific embodiment, M1 and M2 are different, and the antigen-binding protein lacks a non-native T-cell epitope in a C_{H}3 domain of M1 and M2. In one embodiment, the C_{H}3 region of M1 and M2 are each non-immunogenic or substantially non-immunogenic in a human. In a specific embodiment, a modification that reduces or eliminates binding of a heavy chain constant domain to protein A in M1 or M2 does not result in a non-native human T-cell epitope.

In one embodiment, the antigen-binding protein comprises a heavy chain, wherein the heavy chain is non-immunogenic or substantially non-immunogenic in a human. In one embodiment, the heavy chain has an amino acid sequence that does not contain a non-native T cell epitope. In one embodiment, the heavy chain comprises an amino acid sequence whose proteolysis cannot form an amino acid sequence of about 9 amino acids that is immunogenic in a human. In a specific embodiment, the human is a human being treated with the antigen-binding protein. In one embodiment, the heavy chain comprises an amino acid sequence whose proteolysis cannot form an amino acid sequence of about 13 to about 17 amino acids that is immunogenic in a human. In a specific embodiment, the human is a human being treated with the antigen-binding protein.

In one aspect, a multivalent antigen-binding protein comprising a C_{H}2 and/or C_{H}3 modification as described herein is provided, wherein the multivalent binding protein comprises a first immunoglobulin heavy chain variable domain (or V_{H}/V_{L} or scFv) that specifically recognizes an antigen on a B cell, and a second immunoglobulin heavy chain variable domain (or V_{H}/V_{L} or scFv) that specifically recognizes an antigen on a T cell.

In one embodiment, the binding protein is tri-valent. In a specific embodiment, the binding protein comprises an M1 (or an M2, but not both) comprising a human IgG1 heavy chain sequence and an M2 (or an M1, but not both) comprising a human IgG1ΔAdp heavy chain sequence. In one embodiment, the first V_{H} is a human heavy chain variable domain that specifically recognizes CD20. In one embodiment, the second V_{H} is a human heavy chain variable domain that specifically recognizes CD3.

In various aspects, multimerizing components that comprise an immunoglobulin domain selected from a hinge region, a C_{H}2 domain, a C_{H}3 domain, and a combination thereof are provided. In a specific embodiment, a multimerizing component is provided that comprises a hinge, a C_{H}2, and a C_{H}3 domain independently selected from one or more of human IgG1, human IgG2, human IgG3, and human IgG4. In one embodiment, the multimerizing component comprises an immunoglobulin C_{H}2 domain, an immunoglobulin C_{H}3 domain, and optionally a hinge domain, wherein the multimerizing component comprises a modification selected from a modification that reduces or eliminates binding to protein A, a modification that reduces or eliminates an Fc effector function, and a combination thereof.

For multimerizing components that comprise an Fc, e.g., a human Fc, multivalent antigen-binding proteins can be designed to exhibit desired functions mediated by selection of the Fc and/or modification of, e.g., a human Fc. For example, human IgG Fc regions mediate effector functions, such as ADCC. In ADCC, the Fc binds to FcγR on immune cells and mediates cell killing. Different IgG isoforms exert different levels of effector function, and isoforms can be designed or selected according to desirable function.

Human IgG Fc hinge sequences, for example, mediate affinity of IgG for FcγR1 (see, e.g., Canfield, S. M. and Morrison, S. L. (1991) The binding affinity of human IgG for its high affinity Fc receptor is determined by multiple amino acids in the C_{H}2 domain and is modulated by the hinge region, J. Exp. Med. 173:1483-1491). The IgG Fc hinge sequences that mediate affinity of IgG for FcγR1 are conserved among human IgG1, murine IgG2a, and rabbit IgG and consist of Leu-Leu-Gly-Pro-Ser (EU numbering 234-239) (Id.). Mutational studies of IgGs have identified several areas of Fc moieties that are implicated in FOR binding, e.g., in both ends of the C_{H}2 domain sequence. Modifications of Fc domains can include those that reduce or eliminate effector functions without, e.g., affecting pharmacokinetic behavior.

Some IgG effector functions are undesirable or unnecessary for therapeutic activity, such those of human IgG2, wherein disulfide-mediated heterogeneity of human IgG2 antibodies can impact both structure and function. For example, the impact of structural differences on biological activity for a particular human IgG2 was observed to depend on binding affinity, cell surface density of the receptor, and cooperative receptor binding through both Fab domains.

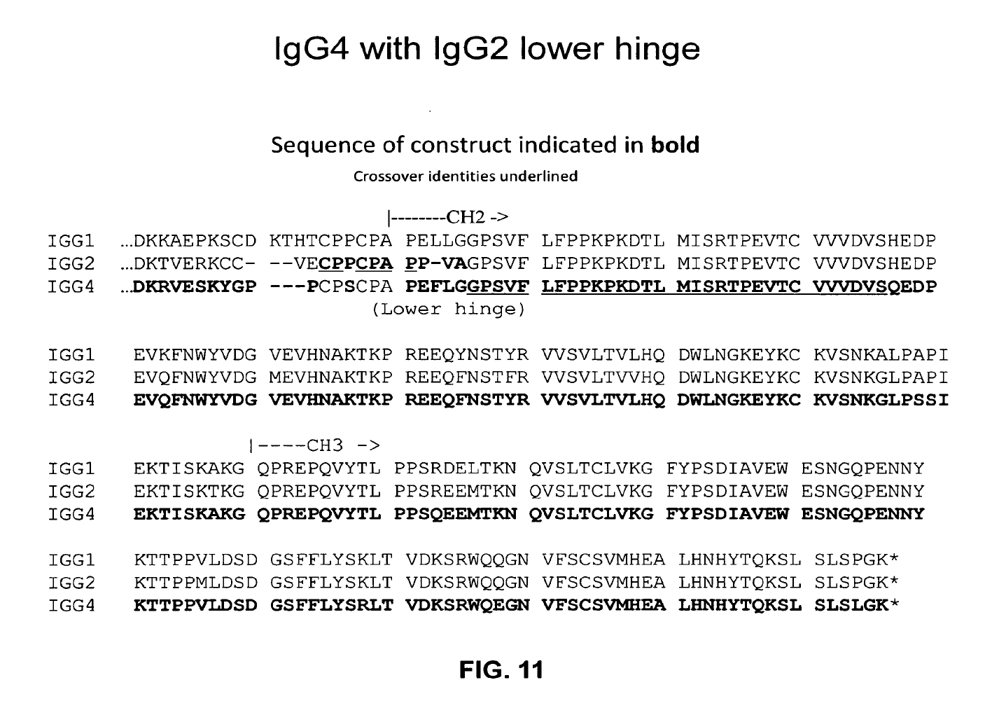

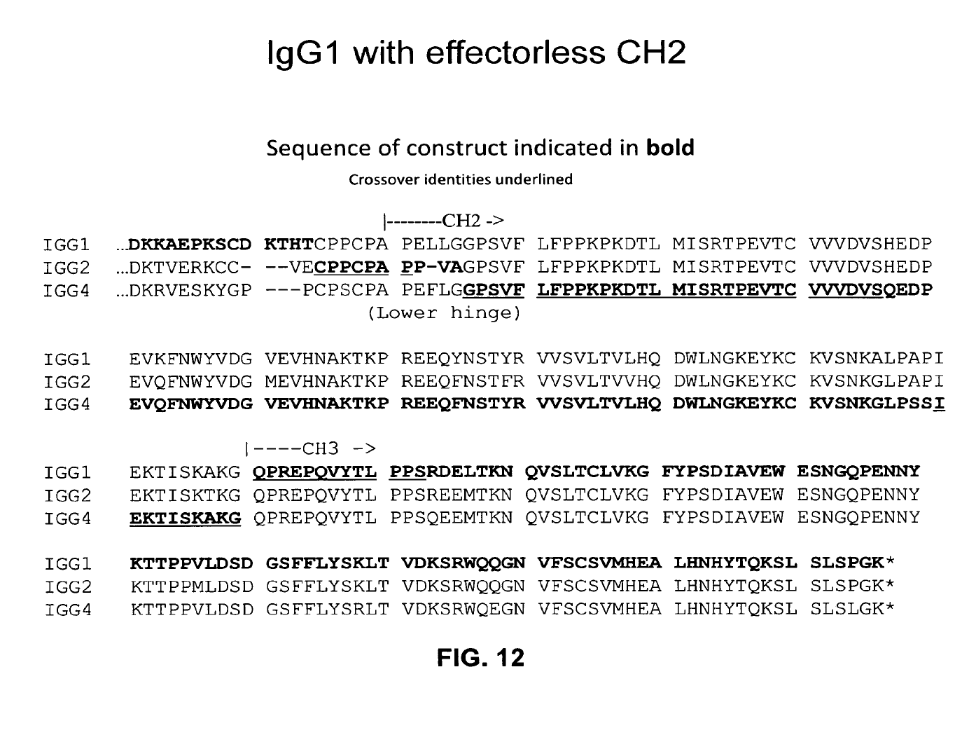

In various embodiments, multimerizing components that comprise (human) IgG moieties are provided that are modified as shown in FIG. 11 and FIG. 12. In one embodiment, a human IgG4 moiety is modified to change the sequence of the lower hinge area from CPSCPAPEFLG to CPPCPAPPVA. In one embodiment, a modified human IgG4 comprising an IgG2 lower hinge region comprises SEQ ID NO: 5. In one embodiment, a human IgG1 moiety is modified to change a sequence of the lower hinge area from CPPCPAPELLG to CPPCPAPPVA, and to replace the IgG1 C_{H}2 domain with an IgG4 C_{H}2 domain. In one embodiment, a modified human IgG1 comprising an effectorless CH2 domain comprises SEQ ID NO: 4).

In some embodiments, the antigen-biding protein as described herein comprises a heavy chain constant (C_{H}) region comprising, from N-terminus to C-terminus, a C_{H}1 domain, a chimeric hinge, a C_{H}2 domain, and a C_{H}3 domain wherein the C_{H}1 domain comprises the amino acid sequence DKKV or DKRV from positions 212 to 215 (EU numbering), the chimeric hinge comprises a human IgG1 or a human IgG4 upper hinge amino acid sequence from positions 216 to 227 (EU numbering) and a human IgG2 lower hinge amino acid sequence PCPAPPVA from positions 228 to 236 (EU numbering), the CH2 domain comprises a human IgG4 CH2 domain amino acid sequence from positions 237 to 340 (EU numbering), and the CH3 domain comprises a human IgG1 or a human IgG4 CH3 domain sequence from positions 341 to 447 (EU numbering). In various embodiments, the C_{H}1 domain comprises the amino acid sequence DKKV, and the chimeric hinge comprises the amino acid sequence EPKSCDKTHTCPPCPAPPVA. In some embodiments, the CH1 domain comprises the amino acid sequence DKRV, and the chimeric hinge comprises the amino acid sequence ESKYGPPCPPCPAPPVA.

In various embodiments, the antigen-binding protein comprising the modifications in the heavy chain constant region as described herein exhibits decreased effector functions when compared to a corresponding antibody comprising the wild-type IgG1 or IgG4 heavy chain constant region, at an antibody concentration of at least 10 nM, wherein the antigen-binding protein exhibits decreased binding, cytotoxic activity, and cellular proliferation.

In various embodiments, the antigen-binding protein comprising the modifications in the heavy chain constant region as described herein exhibits a direct cytotoxic activity of less than about 20%, at an antibody concentration of at least 10 nM. In some embodiments, the antigen-binding protein comprising the modifications in the heavy chain constant region as described herein exhibits a direct cytotoxic activity of less than about 10%, or less than about 5%, or even undetectable, at an antibody concentration of at least 10 nM. In some embodiments, the cytotoxic activity is at least about 10-fold less than the cytotoxic activity of a corresponding antibody comprising a wild-type IgG1 or wild-type IgG4 heavy chain constant region. In some embodiments, the cytotoxic activity is at least about 50-fold less, or about 100-fold less, or about 1000-fold less than the cytotoxic activity of a corresponding antibody comprising a wild-type IgG1 or wild-type IgG4 heavy chain constant region.

We claim:

1. A multivalent antigen-binding protein comprising no more than three immunoglobulin heavy chain variable domains, wherein no more than one of the no more than three immunoglobulin variable domains specifically binds an effector cell, and at least one of the three immunoglobulin variable domains binds a target on a cell that is not the same as the effector cell.

2. The multivalent antigen-binding protein of claim 1, wherein the protein lacks an immunoglobulin light chain variable domain.

3. A multivalent antigen-binding protein, comprising:
a first polypeptide that comprises a first immunoglobulin heavy chain variable domain (V_{H}1), a second heavy chain immunoglobulin domain (V_{H}2), a heavy chain C_{H}1 constant domain, and a first multimerizing component M1;
a second polypeptide comprising a third immunoglobulin heavy chain variable domain (V_{H}3), a fourth immunoglobulin heavy chain variable domain (V_{H}4), a heavy chain C_{H}1 constant domain, and a second multimerizing component M2;
a third polypeptide comprising an immunoglobulin light chain variable domain (V_{L}) present in two copies, and immunoglobulin light chain constant domain (C_{L});
wherein the first polypeptide associates with the second polypeptide by association of M1 and M2, and wherein one molecule of the third polypeptide associates with one molecule of the first polypeptide, and a second molecule of the third polypeptide associates with one molecule of the second polypeptide.

4. The multivalent antigen-binding protein of claim 3, which consists essentially of the first polypeptide, the second polypeptide, and two molecules of the third polypeptide.

5. The multivalent antigen-binding protein of claim 3, wherein M1 and M2 each comprise a heavy chain domain selected from a hinge domain, a C_{H}2 domain, a C_{H}3 domain, and a combination thereof.

6. The multivalent antigen-binding protein of claim 3, wherein the first, second, third, and fourth polypeptides are human immunoglobulin sequences.

7. The multivalent antigen-binding protein of claim 3, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

8. The multivalent antigen-binding protein of claim 7, wherein the third polypeptide comprises a light chain constant domain selected from a human Cκ domain and a human Cλ domain.

9. A multivalent antigen-binding protein, comprising:
a first polypeptide that comprises a first immunoglobulin heavy chain variable domain (V_{H}1), a second heavy chain immunoglobulin variable domain (V_{H}2), a heavy chain C_{H}1 constant domain (C_{H}1), and a first multimerizing component (M1);
a second polypeptide that comprises a third immunoglobulin heavy chain variable domain (V_{H}3), a fourth immunoglobulin heavy chain variable domain (V_{H}4), a heavy chain C_{H}1 constant domain (C_{H}1), and a second multimerizing component (M2);
a third polypeptide comprising an immunoglobulin light chain variable domain (V_{L}1) linked directly or through a linker to a second immunoglobulin light chain variable domain (V_{L}2) that is linked to a light chain constant domain (C_{L});
wherein the first polypeptide associates with the second polypeptide by association of M1 and M2, and wherein a first third polypeptide molecule associates with the first polypeptide, and wherein a second third polypeptide molecule associates with the second polypeptide.

10. The multivalent antigen-binding protein of claim 9, wherein V_{L}1 and V_{L}2 are derived from two different immunoglobulin light chain V gene segments.

11. The multivalent antigen-binding protein of claim 9, wherein V_{L}1 and V_{L}2 are derived from the same immunoglobulin light chain V gene segment.

12. The multivalent antigen-binding protein of claim 9, which consists essentially of the first polypeptide, the second polypeptide, and two molecules of the third polypeptide.

13. The multivalent antigen-binding protein of claim 9, wherein M1 and M2 each comprise a heavy chain domain selected from a hinge domain, a C_{H}2 domain, a C_{H}3 domain, and a combination thereof.

14. The multivalent antigen-binding protein of claim 9, wherein the first, second, third, and fourth polypeptides are human immunoglobulin sequences.

15. The multivalent antigen-binding protein of claim 9, wherein each light chain variable domain of the third polypeptide is independently encoded by a rearranged human nucleic acid sequence derived from a human light chain gene segment selected from a human Vκ1-39 gene segment and a human J segment, a human Vλ3-20 gene segment and a human J segment, a human Vλ1-40 gene segment and a human J segment, and a human Vλ2-14 gene segment and a human J segment.

16. The multivalent antigen-binding protein of claim 15, wherein the third polypeptide comprises a light chain constant domain selected from a human Cκ domain and a human Cλ domain.

17. A multivalent antigen-binding protein comprising:
a first polypeptide comprising a first heavy chain variable domain (V_{H}1) fused directly or through a linker to a second heavy chain variable domain (V_{H}2) fused directly or through a linker to a C_{H}1 region that is attached directly or through a linker to a multimerizing component M1;
a second polypeptide comprising a third heavy chain variable domain (V_{H}3) fused directly or through a linker to a C_{H}1 region that is attached directly or through a linker to a multimerizing component M2; and,
a third polypeptide that comprises a first light chain variable domain V_{L} fused directly or through a linker to a second light chain variable domain V_{L} that is fused directly or through a linker to a light chain constant region;
wherein the multivalent antigen-binding protein comprises a heterodimer of the first and the second polypeptide, wherein each of the first and the second polypeptides are associated with one molecule of the third polypeptide.

18. The multivalent antigen-binding protein of claim 17, which consists essentially of three polypeptides, wherein the first polypeptide consists essentially of a first heavy chain immunoglobulin variable domain (V_{H}1) fused directly or through a linker to a second heavy chain immunoglobulin variable domain (V_{H}2) fused directly or through a linker to a C_{H}1 region that is attached directly or through a linker to a multimerizing component M1; a second polypeptide that consists essentially of a third heavy chain immunoglobulin variable domain (V_{H}3) fused directly or through a linker to a C_{H}1 region that is fused directly or through a linker to a multimerizing component M2; and a third polypeptide (present in two copies) consisting essentially of an immunoglobulin light chain variable domain fused directly or through a linker to a second immunoglobulin light chain variable domain, which is in turn fused directly or through a linker to a light chain constant region.

19. The multivalent antigen-binding protein of claim 18, wherein the first and the second immunoglobulin light chain constant domains are cognate to each of V_{H}1, V_{H}2, and V_{H}3.

20. The multivalent antigen-binding protein of claim 17, comprising a polypeptide having a tandem (optionally separated by a linker) pair of immunoglobulin light chain variable domains, one of said pair which is fused (optionally through a linker) to a light chain constant region, wherein each of the pair of immunoglobulin light chain variable domains is cognate with a sequences of a second polypeptide having each of two different heavy chain variable domains V_{H}1 and V_{H}2, wherein one of the heavy chain variable domains is associated with a C_{H}1 region, and the second polypeptide is associated with a multimerizing component; and a third polypeptide comprising a third heavy chain variable domain associated with a C_{H}1 region and a multimerizing component, but not associated with a light chain variable domain.

21. The multivalent antigen-binding protein of claim 20, wherein the unassociated light chain variable domain is a single light chain variable domain that specifically binds an epitope that is not bound by V_{H}1 or V_{H}2, or that is not bound by V_{H}1 or V_{H}2 or V_{H}3.

22. The multivalent antigen-binding protein of claim 17, comprising:
a first polypeptide that consists essentially of a V_{H}1, a V_{H}2, a C_{H}1, an M1;
a second polypeptide that consists essentially of a V_{H}3, a C_{H}1, and a M2;
a third polypeptide comprising an immunoglobulin light chain variable domain (V_{L}) present in two copies (in one embodiment separated by a linker sequence), and an immunoglobulin light chain constant domain (C_{L});
wherein the first polypeptide associates with the second polypeptide by multimerizing components M1 and M2, and wherein one third polypeptide molecule associates with the first polypeptide, and wherein one third polypeptide molecule associates with the second polypeptide.

23. The multivalent antigen-binding protein of claim 17, wherein M1 and M2 each comprise a heavy chain domain selected from a hinge domain, a C_{H}2 domain, a C_{H}3 domain, and a combination thereof.

24. The multivalent antigen-binding protein of claim 17, wherein each polypeptide comprises human immunoglobulin sequences.

25. The multivalent antigen-binding protein of claim 17, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

26. The multivalent antigen-binding protein of claim 17, wherein the third polypeptide comprises a light chain constant domain selected from a human Cκ domain and a human Cλ domain.

27. A multivalent antigen-binding protein comprising:
a first polypeptide that comprises an immunoglobulin single heavy chain variable domain (V_{H}1sd) fused directly or through a linker to a second immunoglobulin heavy chain variable domain (V_{H}2), fused directly or through a linker to a C_{H}1 region, which is fused directly or through a linker or hinge to a multimerizing component M1;
a second polypeptide comprising a third immunoglobulin single heavy chain variable domain (V_{H}3sd) fused directly or through a linker to a fourth immunoglobulin heavy chain variable domain (V_{H}4) that is fused directly or through a linker to a C_{H}1 region, which is fused directly or through a linker to a multimerizing component M2; and,
a third polypeptide comprising a light chain variable domain fused directly or through a linker to a light chain constant region.

28. The multivalent antigen-binding protein of claim 27, wherein the light chain polypeptide consists essentially of a light chain variable domain and a light chain constant region.

29. The multivalent antigen-binding protein of claim 27, wherein V_{H}1sd and V_{H}3sd each respectively bind an antigen in the absence of a cognate light chain variable domain.

30. The multivalent antigen-binding protein of claim 27, which consists essentially of the first polypeptide, the second polypeptide, and two molecules of the third polypeptide.

31. The multivalent antigen-binding protein of claim 27, wherein M1 and M2 each comprise a heavy chain domain selected from a hinge domain, a C_{H}2 domain, a C_{H}3 domain, and a combination thereof.

32. The multivalent antigen-binding protein of claim 27, wherein the first, second, and third polypeptides are human immunoglobulin sequences.

33. The multivalent antigen-binding protein of claim 27, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

34. The multivalent antigen-binding protein of claim 7, wherein the third polypeptide comprises a light chain constant domain selected from a human Cκ domain and a human Cλ domain.

35. A multivalent antigen-binding protein comprising:
a first polypeptide comprising a first immunoglobulin heavy chain single variable domain (V_{L}1sd) fused directly or through a linker to a light chain variable domain (V_{L}2), which is fused directly or through a linker to a light chain constant region;
a second polypeptide comprising (or consisting essentially of) a second heavy chain variable domain (V_{H}2) fused directly or through a linker to a C_{H}1 region, which is fused directly or through a linker to a first multimerizing component M1; and a third polypeptide comprising a fourth heavy chain variable domain (V_{H}4) fused directly or through a linker to a C_{H}1 region that is fused directly or through a linker to a second multimerizing component M2.

36. The multivalent antigen-binding protein of claim 35, which consists essentially of the first polypeptide, the second polypeptide, and two molecules of the third polypeptide.

37. The multivalent antigen-binding protein of claim 35, wherein M1 and M2 each comprise a heavy chain domain selected from a hinge domain, a C_{H}2 domain, a C_{H}3 domain, and a combination thereof.

38. The multivalent antigen-binding protein of claim 35, wherein the first, second, and third polypeptides are human immunoglobulin sequences.

39. The multivalent antigen-binding protein of claim 35, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

40. The multivalent antigen-binding protein of claim 35, wherein the third polypeptide comprises a light chain constant domain selected from a human Cκ domain and a human Cλ domain.

41. A multivalent antigen-binding protein comprising:
a first polypeptide that comprises a first immunoglobulin heavy chain variable domain fused directly or through a linker to a second immunoglobulin heavy chain variable domain, which is fused directly or through a linker to a C_{H}1 region, which is attached directly or through a linker to a first multimerizing component;
a second polypeptide comprising a heavy chain single immunoglobulin variable domain (V_{H}3sd) fused directly or through a linker to a second multimerizing component; and,
a third polypeptide comprising a first light chain variable domain fused directly or through a linker to a second immunoglobulin light chain variable domain (V_{L}2), which is fused directly or through a linker to a light chain constant domain.

42. The multivalent antigen-binding protein of claim 41, which consists essentially of the first polypeptide, the second polypeptide, and two molecules of the third polypeptide.

43. The multivalent antigen-binding protein of claim 41, wherein M1 and M2 each comprise a heavy chain domain selected from a hinge domain, a C_{H}2 domain, a C_{H}3 domain, and a combination thereof.

44. The multivalent antigen-binding protein of claim 41, wherein the first, second, and third polypeptides are human immunoglobulin sequences.

45. The multivalent antigen-binding protein of claim 41, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

46. The multivalent antigen-binding protein of claim 41, wherein the third polypeptide comprises a light chain constant domain selected from a human Cκ domain and a human Cλ domain.

47. The multivalent antigen-binding protein of claim 41, wherein the V_{H}1 and V_{H}2 are different.

48. A multivalent antigen-binding protein comprising:
a first polypeptide that comprises a first single immunoglobulin heavy chain variable domain (V_{H}1sd), a second heavy chain single immunoglobulin variable domain (V_{H}2sd), and a first multimerizing component (M1), wherein the first polypeptide lacks an immunoglobulin C_{H}1 domain;
a second polypeptide that comprises a third single immunoglobulin heavy chain variable domain (V_{H}3sd), a fourth immunoglobulin heavy chain variable domain (V_{H}4sd) and a second multimerizing component (M2), wherein the second polypeptide lacks an immunoglobulin C_{H}1 domain;
wherein the first polypeptide associates with the second polypeptide by multimerizing components M1 and M2.

49. The multivalent antigen-binding protein of claim 48, which consists essentially of two polypeptides, wherein the first polypeptide consists essentially of two heavy chain single immunoglobulin variable domains (V_{H}1sd and V_{H}2sd) and a multimerizing component M1; and wherein the second polypeptide consists essentially of two heavy chain single immunoglobulin variable domains (V_{H}3sd and V_{H}4sd) and a multimerizing component M2.

50. The multivalent antigen-binding protein of claim 48, wherein V_{H}1sd, V_{H}2sd, V_{H}3sd, and V_{H}4sd bind to at least one, at least two, at least three, or four epitopes.

51. A multivalent antigen-binding protein comprising a first multimerizing component M1 fused with two single light chain immunoglobulin variable domains, and a second multimerizing component M2 fused with one or two single light chain immunoglobulin variable domains.

52. The multivalent antigen-binding protein of claim 51, wherein the second multimerizing component M2 is fused with two single light chain immunoglobulin variable domains.

53. The multivalent antigen-binding protein of claim 51, which consists essentially of a first and a second polypeptide, wherein:
the first polypeptide consists essentially of a first multimerizing component M1 fused directly or through a linker with a first single domain light chain immunoglobulin variable domain that is attached directly or through a linker to a second single domain light chain immunoglobulin variable domain; and,
the second polypeptide consists essentially of a second multimerizing component M2 fused directly or through a linker to a fourth single domain light chain variable domain that is fused directly or through a linker to a third single domain light chain variable domain.

54. A multivalent antigen-binding protein comprising:
a first polypeptide that comprises a first immunoglobulin heavy chain variable domain (V_{H}1), a second heavy chain immunoglobulin variable domain (V_{H}2), and a first multimerizing component (M1), wherein the first polypeptide lacks an immunoglobulin C_{H}1 domain;
a second polypeptide that comprises a third immunoglobulin heavy chain variable domain (V_{H}3), and a second multimerizing component (M2), wherein the second polypeptide lacks an immunoglobulin C_{H}1 domain;
wherein the first polypeptide associates with the second polypeptide by multimerizing components M1 and M2, and wherein the antigen-binding protein lacks an immunoglobulin light chain variable domain and lacks an immunoglobulin light chain constant domain.

55. The multivalent antigen-binding protein of claim 54, which consists essentially of the first polypeptide and the second polypeptide.

56. The multivalent antigen-binding protein of claim 54, wherein M1 and M2 each comprise a heavy chain domain selected from a hinge domain, a C_{H}2 domain, a C_{H}3 domain, and a combination thereof.

57. The multivalent antigen-binding protein of claim 54, wherein the first and second polypeptides are human immunoglobulin sequences.

58. A multivalent antigen-binding protein that binds three different antigens by three different heavy chain immunoglobulin single variable domains, wherein:
a first and a second heavy chain immunoglobulin single variable domain are disposed on a first multimerizing component M1;
the third heavy chain immunoglobulin single variable domain is disposed on a second multimerizing component M2;
wherein the antigen-binding protein consists essentially of a first polypeptide and a second polypeptide, wherein the first polypeptide consists essentially of a first heavy chain immunoglobulin single variable domain (V_{H}1sd) fused directly or through a linker to a second heavy chain immunoglobulin singe variable domain (V_{H}2sd) fused directly or through a linker to a first multimerizing component M1; and the second polypeptide consists essentially of a third heavy chain immunoglobulin single variable domain fused directly or through a linker to a second multimerizing component M2.

59. The multivalent antigen-binding protein of claim 58, wherein the first, the second, and the third single variable domain bind three different epitopes.

60. The multivalent antigen-binding protein of claim 58, which consists essentially of the first polypeptide and the second polypeptide.

61. The multivalent antigen-binding protein of claim 58, wherein M1 and M2 each comprise a heavy chain domain selected from a hinge domain, a C_{H}2 domain, a C_{H}3 domain, and a combination thereof.

63. The multivalent antigen-binding protein of claim 58, wherein the first heavy chain immunoglobulin single variable domain and the second heavy chain immunoglobulin single variable domain and the third heavy chain immunoglobulin single variable domain are each human immunoglobulin heavy chain variable domains.

64. A multivalent antigen-binding protein comprising:
a first polypeptide that comprises a first immunoglobulin single light chain variable domain fused directly or through a linker to a second immunoglobulin single light chain variable domain, which is fused directly or through a linker to a first multimerizing component M1, wherein the first polypeptide is not associated with an immunoglobulin heavy chain variable domain;
a second polypeptide that comprises a third immunoglobulin single light chain variable domain fused directly or through a linker to a second multimerizing component M2, wherein the second polypeptide is not associated with an immunoglobulin heavy chain variable domain;
wherein the multivalent antigen-binding protein lacks an immunoglobulin light chain constant region domain.

65. The multivalent antigen-binding protein of claim 64, which consists essentially of the first polypeptide and the second polypeptide.

66. The multivalent antigen-binding protein of claim 64, wherein M1 and M2 each comprise a heavy chain domain selected from a hinge domain, a C_{H}2 domain, a C_{H}3 domain, and a combination thereof.

67. The multivalent antigen-binding protein of claim 64, wherein the first, the second, and the third immunoglobulin light chain single variable domains are human immunoglobulin sequences.

68. The multivalent antigen-binding protein of claim 64, wherein the first, the second, and the third immunoglobulin light chain single variable domains are selected from a human κ light chain variable domain, a human λ light chain variable domain, and a combination thereof.

69. A multivalent antigen-binding protein comprising:
a first polypeptide that comprises a first scFv (scFv1), a second scFv (scFv2), optionally a linker, and a first multimerizing component (M1); and,
a second polypeptide that comprises a third scFv (scFv3), optionally a linker, and a second multimerizing component (M2).

70. The multivalent antigen-binding protein of claim 69, which consists essentially of the first polypeptide and the second polypeptide.

71. The multivalent antigen-binding protein of claim 69, wherein M1 and M2 each comprise a heavy chain domain selected from a hinge domain, a C_{H}2 domain, a C_{H}3 domain, and a combination thereof.

72. The multivalent antigen-binding protein of claim 69, wherein the first and second scFv comprise human immunoglobulin variable domains, and M1 and M2 are each human sequences.

73. A multivalent antigen-binding protein comprising:
a first polypeptide that comprises a first immunoglobulin heavy chain variable domain (V_{H}1), a heavy chain C_{H}1 constant domain (C_{H}1), a first multimerizing component (M1), a second C_{H}1, a second heavy chain immunoglobulin variable domain (V_{H}2);
a second polypeptide that comprises a third immunoglobulin heavy chain variable domain (V_{H}3), a C_{H}1, a second multimerizing component (M2), a C_{H}1, and a fourth immunoglobulin heavy chain variable domain (C_{H}4);
a third polypeptide comprising an immunoglobulin light chain variable domain (V_{L}) and an immunoglobulin light chain constant domain (C_{L});
wherein the first polypeptide associates with the second polypeptide by multimerizing components M1 and M2, and wherein two third polypeptide molecules associate with the first polypeptide, and wherein two third polypeptide molecules associates with the second polypeptide.

74. The multivalent antigen-binding protein of claim 73, which consists essentially of six polypeptides, wherein:
the first polypeptide consists essentially of (from N-terminal to C-terminal), a first immunoglobulin heavy chain variable domain (V_{H}1) fused directly or through a linker to a first C_{H}1 to form a V_{H}1-C_{H}1 region, which is fused directly or through a linker to a first multimerizing component (M1), which is fused directly or through a linker to a second immunoglobulin heavy chain variable domain (V_{H}2) which is in turn fused directly or through a linker to a second C_{H}1 region to form a V_{H}2-C_{H}1 region;
the V_{H}1-C_{H}1 region is associated with a second polypeptide that consists essentially of a cognate (with respect to V_{H}1) immunoglobulin light chain variable domain fused directly or through a linker to a light chain constant domain;
the V_{H}2-C_{H}1 region is associated with a third polypeptide that consists essentially of a cognate (with respect to V_{H}2) immunoglobulin light chain domain fused directly or through a linker to a light chain constant domain;
the fourth polypeptide consists essentially of (from N-terminal to C-terminal) a third immunoglobulin heavy chain variable domain (V_{H}3) fused directly or through a linker to a C_{H}1 region, which is in turn fused directly or through a linker to a second multimerizing component M2, which is fused directly or through a linker to a fourth immunoglobulin heavy chain variable domain (V_{H}4) fused directly or through a linker to a C_{H}1 region;
a fifth polypeptide consisting essentially of an immunoglobulin light chain variable domain that is cognate with respect to V_{H}3 fused directly or through a linker to a light chain constant region is associated with V_{H}3; and,
a sixth polypeptide consisting essentially of an immunoglobulin light chain variable domain that is cognate with respect to V_{H}4 is fused directly or through a linker to a light chain constant region and is associated with V_{H}4.

75. The multivalent antigen-binding protein of claim 73, which consists essentially of the first polypeptide, the second polypeptide, the third polypeptide, the fourth polypeptide, the fifth polypeptide, and the sixth polypeptide.

76. The multivalent antigen-binding protein of claim 73, wherein M1 and M2 each comprise a heavy chain domain selected from a hinge domain, a C_{H}2 domain, a C_{H}3 domain, and a combination thereof.

77. The multivalent antigen-binding protein of claim 73, wherein each immunoglobulin sequence is a human immunoglobulin sequence.

78. The multivalent antigen-binding protein of claim 73, wherein the light chain variable domains are encoded by a rearranged human nucleic acid sequence derived from a human light chain gene segment selected from a human Vλ1-39 gene segment and a human J segment, a human Vκ3-20 gene segment and a human J segment, a human Vλ1-40 gene segment and a human J segment, and a human Vλ2-14 gene segment and a human J segment, and a combination thereof.

79. The multivalent antigen-binding protein of claim 73, wherein the light chain constant domains are selected from a human Cκ domain, and a human Cλ domain, and a combination thereof.

80. The multivalent antigen-binding protein of claim 73, wherein the immunoglobulin light chains are encoded by a nucleic acid sequence derived from a rearrangement of the same light chain variable region gene segment and the same light chain J segment to form a rearranged light chain variable region gene sequence, and the immunoglobulin light chains each comprise the same light chain constant region.

81. The multivalent antigen-binding protein of claim 80, wherein the rearranged light chain variable region gene sequence is derived from a Vκ1-39/J rearrangement, a Vκ3-20/J rearrangement, a Vλ1-40/J rearrangement, or a Vλ2-14/J rearrangement.

82. A multivalent antigen-binding protein comprising:
a first polypeptide that comprises a first immunoglobulin heavy chain variable domain (V_{H}1), a heavy chain C_{H}1 constant domain (C_{H}1), a first multimerizing component (M1), a second C_{H}1, a second heavy chain immunoglobulin variable domain (V_{H}2);
a second polypeptide that comprises a third immunoglobulin heavy chain variable domain (V_{H}3), a C_{H}1, and a second multimerizing component (M2);
a third polypeptide comprising an immunoglobulin light chain variable domain (V_{L}) and an immunoglobulin light chain constant domain (C_{L}) (i.e., V_{L}-C_{L});
wherein the first polypeptide associates with the second polypeptide by multimerizing components M1 and M2, and wherein two third polypeptide molecules associate with the first polypeptide, and wherein one third polypeptide molecule associates with the second polypeptide.

83. The antigen-binding protein of claim 82, which consists essentially of five polypeptides, wherein:
the first polypeptide consists essentially of (from N-terminal to C-terminal) a first heavy chain immunoglobulin variable domain (V_{H}1) fused directly or through a linker to a first C_{H}1, which is in turn fused directly or through a linker to a first multimerizing component M1, which is fused directly or through a linker to a second heavy chain immunoglobulin variable domain that is fused directly or through a linker to a second C_{H}1 sequence;
a second polypeptide that consists essentially of (from N-terminal to C-terminal) a third immunoglobulin heavy chain variable domain (V_{H}3) fused directly or through a linker to a third C_{H}1 region, which in turn is fused directly or through a linker to a second multimerizing component M2;
a third polypeptide that consists essentially of a first light chain variable domain that is cognate with V_{H}1 and that is fused directly or through a linker to a first light chain constant region, wherein the third polypeptide is associated with V_{H}1-C_{H}1;
a fourth polypeptide that consists essentially of a second light chain variable domain that is cognate with V_{H}2, and that is fused directly or through a linker to a second light chain constant region, wherein the fourth polypeptide is associated with V_{H}2-C_{H}1; and,
a fifth polypeptide that consists essentially of a third light chain variable domain that is cognate with V_{H}3, and that is fused directly or through a linker to a third light chain constant region, wherein the fifth polypeptide is associated with V_{H}3-C_{H}1.

84. The multivalent antigen-binding protein of claim 83, wherein V_{H}1, V_{H}2, and V_{H}3 each specifically bind a different antigen.

85. The multivalent antigen-binding protein of claim 84, wherein V_{H}1, V_{H}2, and V_{H}3 bind no more than two antigens, optionally two or more of V_{H}1, V_{H}2, and V_{H}3 bind different epitopes of a same antigen.

86. The multivalent antigen-binding protein of claim 83, which consists essentially of the first polypeptide, the second polypeptide, and three molecules of the third polypeptide.

87. The multivalent antigen-binding protein of claim 83, wherein M1 and M2 each comprise a heavy chain domain selected from a hinge domain, a C_{H}2 domain, a C_{H}3 domain, and a combination thereof.

88. The multivalent antigen-binding protein of claim 83, wherein the first, second, and third polypeptides comprise human immunoglobulin variable domains and human immunoglobulin constant domains.

89. The multivalent antigen-binding protein of claim 83, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vκ2-14 gene segment and a human J segment.

90. The multivalent antigen-binding protein of claim 83, wherein the third polypeptide comprises a light chain constant domain selected from a human Cκ domain and a human Cλ domain.

91. A multispecific antigen-binding protein comprising three polypeptides, wherein:
the first polypeptide comprises (from N-terminal to C-terminal) a first heavy chain immunoglobulin variable domain (V_{H}1) fused directly or through a linker to a first C_{H}1 region, which in turn is fused directly or through a linker to a first multimerizing component M1, which is in turn fused directly or through a linker to a second heavy chain immunoglobulin variable domain that is a heavy chain single immunoglobulin variable domain (V_{H}2sd);
a second polypeptide comprising (from N-terminal to C-terminal) a third heavy chain immunoglobulin variable domain (V_{H}3) fused directly or through a linker to a C_{H}1 region, which in turn is fused directly or through a linker to a second multimerizing component;
a third polypeptide comprising a first light chain variable domain that is cognate with V_{H}1, wherein the first light chain variable domain is fused directly or through a linker to a light chain constant domain; and,
a fourth polypeptide comprising a second light chain variable domain that is cognate with V_{H}3, wherein the second light chain variable domain is fused directly or through a linker to a second light chain constant domain.

92. The antigen-binding protein of claim 91, which consists essentially of four polypeptides, wherein:
the first polypeptide consists essentially of first heavy chain immunoglobulin variable domain V_{H}1 fused directly or through a linker to a first C_{H}1 region, which in turn is fused directly or through a linker to a first multimerizing component M1, which is in turn fused directly or through a linker to a second heavy chain immunoglobulin variable domain that is a heavy chain single immunoglobulin variable domain (V_{H}2sd);
a second polypeptide that consists essentially of a third heavy chain immunoglobulin variable domain fused directly or through a linker to a C_{H}1, which in turn is fused directly or through a linker to a second multimerizing component M2; and,
a third polypeptide that consists essentially of a light chain variable domain that is cognate with V_{H}1, and is associated with a light chain constant region; and a fourth polypeptide that consists essentially of a light chain variable domain that is cognate with V_{H}3, and is associated with a light chain constant region.

93. The multivalent antigen-binding protein of claim 91, which consists essentially of the first polypeptide, the second polypeptide, and two molecules of the third polypeptide.

94. The multivalent antigen-binding protein of claim 91, wherein M1 and M2 each comprise a heavy chain domain selected from a hinge domain, a C_{H}2 domain, a C_{H}3 domain, and a combination thereof.

95. The multivalent antigen-binding protein of claim 91, wherein all the immunoglobulin variable domains and constant domains are human.

96. The multivalent antigen-binding protein of claim 91, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

97. The multivalent antigen-binding protein of claim 91, wherein the third polypeptide comprises a light chain constant domain selected from a human Cκ domain and a human Cλ domain.

98. A multivalent antigen-binding protein comprising four polypeptides, wherein:
the first polypeptide comprises (from N-terminal to C-terminal) a first heavy chain immunoglobulin variable domain (V_{H}1) fused directly or through a linker to a first C_{H}1 sequence, which is fused directly or through a linker to a first multimerizing component M1, which in turn is fused with a light chain immunoglobulin single variable domain (V_{L}2sd);
a second polypeptide (from N-terminal to C-terminal) comprising a third immunoglobulin variable domain that is a heavy chain immunoglobulin variable domain (V_{H}3) fused directly or through a linker to a second C_{H}1 region, which in turn is fused directly or through a linker to a second multimerizing component M2;
a third polypeptide that comprises a light chain variable domain (V_{L}) that is cognate with V_{H}1, and that is associated with a light chain constant region (C_{L}); and,
a fourth polypeptide that comprises a light chain variable domain (V_{L}) that is cognate with V_{H}3, and that is associated with a light chain variable domain.

99. The multivalent antigen-binding protein of claim 98, which consists essentially of four polypeptides, wherein:
the first polypeptide consists essentially of a first heavy chain immunoglobulin variable domain (V_{H}1) associated with a first C_{H}1 sequence, which is fused directly or through a linker to a first multimerizing component M1, which in turn is fused with a light chain immunoglobulin single variable domain (V_{L}2sd);
a second polypeptide consisting essentially of (from N-terminal to C-terminal) a third immunoglobulin variable domain that is a heavy chain immunoglobulin variable domain (V_{H}3) fused directly or through a linker to a second C_{H}1 region, which in turn is fused directly or through a linker to a second multimerizing component M2;
a third polypeptide consisting essentially of a light chain variable domain (V_{L}) that is cognate with V_{H}1, and that is associated with a light chain constant region (C_{L}); and, a fourth polypeptide that consists essentially of a light chain variable domain (V_{L}) that is cognate with VH3 and that is associated with a light chain variable domain.

100. The multivalent antigen-binding protein of claim 98, which consists essentially of the first polypeptide, the second polypeptide, and two molecules of the third polypeptide.

101. The multivalent antigen-binding protein of claim 98, wherein M1 and M2 each comprise a heavy chain domain selected from a hinge domain, a C_{H}2 domain, a C_{H}3 domain, and a combination thereof.

102. The multivalent antigen-binding protein of claim 98, wherein the first, second, third, and fourth polypeptides contain immunoglobulin variable and constant sequences that are human immunoglobulin sequences.

103. The multivalent antigen-binding protein of claim 98, wherein the light chain variable domain of the third polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

104. The multivalent antigen-binding protein of claim 98, wherein the third polypeptide comprises a light chain constant domain selected from a human Cκ domain and a human Cλ domain.

105. A multivalent antigen-binding protein is provided, comprising four polypeptides, wherein:
the first polypeptide comprises (from N-terminal to C-terminal) a first immunoglobulin heavy chain variable domain fused directly or through a linker to a first C_{H}1, which is fused directly or through a linker to a first multimerizing component M1, which is in turn fused directly or through a linker to a light chain single immunoglobulin variable domain (V_{L}2sd);
a second polypeptide that comprises (from N-terminal to C-terminal) an immunoglobulin heavy chain variable domain (V_{H}3) fused directly or through a linker to a second C_{H}1, which in turn is fused directly or through a linker to a second multimerizing component M2, which in turn is fused directly or through a linker to an immunoglobulin single light chain variable domain (V_{L}3sd);
a third polypeptide that comprises a light chain variable domain (V_{L}1) that is cognate with V_{H}1 and is associated with a light chain constant region; and,
a fourth polypeptide that comprises a light chain variable domain (V_{L}1) that is cognate with V_{H}3 and is associated with a light chain constant region.

106. The multivalent antigen-binding protein of claim 105, which consists essentially of four polypeptides, wherein:
the first polypeptide consists essentially of (from N-terminal to C-terminal) a first immunoglobulin heavy chain variable domain (V_{H}1) associated with a first C_{H}1, which is fused directly or through a linker to a first multimerizing component M1, which is in turn fused to a light chain immunoglobulin single variable domain (V_{L}2sd) that binds its target in the absence of a cognate heavy chain;
a second polypeptide consisting essentially of (from N-terminal to C-terminal) an immunoglobulin heavy chain variable domain (V_{H}3) associated with a second C_{H}1 that is fused directly or through a linker to a second multimerizing component M2, which in turn is fused to a light chain immunoglobulin single variable domain (V_{L}3sd) that binds its target in the absence of a cognate heavy chain;
a third polypeptide that consists essentially of a light chain immunoglobulin variable domain (V_{L}1) that is cognate with V_{H}1 and is associated with a light chain constant region (C_{L}); and,
a fourth polypeptide that consists essentially of a light chain immunoglobulin variable domain that is cognate with V_{H}3 and is associated with a light chain constant region.

107. The multivalent antigen-binding protein of claim 105, which consists essentially of the first polypeptide, the second polypeptide, and two molecules of the third polypeptide.

108. The multivalent antigen-binding protein of claim 105, wherein M1 and M2 each comprise a heavy chain domain selected from a hinge domain, a C_{H}2 domain, a C_{H}3 domain, and a combination thereof.

109. The multivalent antigen-binding protein of claim 105, wherein the first, second, third, and fourth polypeptides contain immunoglobulin variable and constant domains that are human.

110. The multivalent antigen-binding protein of claim 105, wherein the light chain variable domain of the third polypeptide is encoded by a rearranged human nucleic acid sequence derived from a human light chain gene segment selected from a human Vκ1-39 gene segment and a human J segment, a human Vλ3-20 gene segment and a human J segment, a human Vλ1-40 gene segment and a human J segment, and a human Vλ2-14 gene segment and a human J segment.

111. The multivalent antigen-binding protein of claim 105, wherein the third polypeptide comprises a light chain constant domain selected from a human Cκ domain and a human Cλ domain.

112. A multivalent antigen-binding protein comprising a first polypeptide and a second polypeptide, wherein:
the first polypeptide comprises a first immunoglobulin heavy chain variable domain (V_{H}1), optionally a linker, a first multimerizing component (M1), optionally a linker, and a second immunoglobulin heavy chain variable domain; and,
the second polypeptide comprises a third immunoglobulin heavy chain variable domain (V_{H}3), optionally a linker, a second multimerizing component (M2), optionally a linker, and a fourth heavy chain variable domain (V_{H}4);
wherein the antigen-binding protein lacks a C_{H}1, and lacks an immunoglobulin light chain variable domain.

113. The multivalent antigen-binding protein of claim 112, which consists essentially of two polypeptides, wherein:
the first polypeptide consists essentially of a first heavy chain immunoglobulin single variable domain (V_{H}1sd) fused directly or through a linker to a first multimerizing component M1, which in turn is fused directly or through a linker to a second heavy chain immunoglobulin single variable domain (V_{H}2sd); and,
the second polypeptide consists essentially of a third heavy chain immunoglobulin single variable domain (V_{H}3sd) fused directly or through a linker to a second multimerizing component M2, which in turn is fused directly or through a linker to a fourth heavy chain immunoglobulin single variable domain (V_{H}4sd).

114. The multivalent antigen-binding protein of claim 112, which consists essentially of the first polypeptide and the second polypeptide.

115. The multivalent antigen-binding protein of claim 112, wherein M1 and M2 each comprise a heavy chain domain selected from a hinge domain, a C_{H}2 domain, a C_{H}3 domain, and a combination thereof.

116. The multivalent antigen-binding protein of claim 112, wherein each heavy chain immunoglobulin single variable domain is a human immunoglobulin variable domain.

117. A multivalent antigen-binding protein that comprises a first polypeptide and a second polypeptide, wherein:
the first polypeptide comprises (from N-terminal to C-terminal) a first light chain immunoglobulin single variable domain (V_{L}sd1) fused directly or through a linker to a first multimerizing component M1, which in turn is fused directly or through a linker to a second light chain immunoglobulin single variable domain (V_{L}sd2); and,
the second polypeptide comprises (from N-terminal to C-terminal) a third light chain immunoglobulin single variable domain (V_{L}sd3) fused directly or through a linker to a second multimerizing component M2, which in turn is fused directly or through a linker to a fourth light chain immunoglobulin single variable domain (V_{L}sd4).

118. The multivalent antigen-binding protein of claim 117, wherein V_{L}sd1, V_{L}sd2, V_{L}sd3, V_{L}sd4 each bind a different epitope.

119. The multivalent antigen-binding protein of claim 117, wherein V_{L}sd1, V_{L}sd2, V_{L}sd3, and V_{L}sd4 bind no more than three antigens; optionally at least two of V_{L}sd1, V_{L}sd2, V_{L}sd3, or V_{L}sd4 bind the same or a different epitope on a first same antigen, and the remaining two each bind a separate antigen that is not the first same antigen.

120. The multivalent antigen-binding protein of claim 117, which consists essentially of a first polypeptide and a second polypeptide, wherein:
the first polypeptide consists essentially of (from N-terminal to C-terminal) a first light chain immunoglobulin single variable domain (V_{L}sd1) fused directly or through a linker to a first multimerizing component M1, fused directly or through a linker to a second light chain immunoglobulin single variable domain (V_{L}sd2); and,
the second polypeptide consisting essentially of a third light chain immunoglobulin single variable domain (V_{L}sd3) fused directly or through a linker to a second multimerizing component M2, which is fused directly or through a linker to a fourth light chain immunoglobulin single variable domain (V_{L}sd4).

121. The multivalent antigen-binding protein of claim 117, which consists essentially of the first polypeptide and the second polypeptide

122. The multivalent antigen-binding protein of claim 117, wherein M1 and M2 each comprise a heavy chain domain selected from a hinge domain, a C_{H}2 domain, a C_{H}3 domain, and a combination thereof.

123. The multivalent antigen-binding protein of claim 117, wherein the first, second, third, and fourth light chain immunoglobulin single variable domains are human.

124. The multivalent antigen-binding protein of claim 117, wherein antigen-binding protein lacks a light chain immunoglobulin constant domain.

125. A multivalent antigen-binding protein comprising:
a first polypeptide that comprises a first immunoglobulin heavy chain variable domain (V_{H}1), optionally a linker, a first multimerizing component (M1), optionally a linker, and a second immunoglobulin heavy chain variable domain; and,
a second polypeptide that comprises (or consists essentially of) a third immunoglobulin heavy chain variable domain (V_{H}3), optionally a linker, a second multimerizing component (M2);
wherein the antigen-binding protein lacks a C_{H}1, and lacks an immunoglobulin light chain variable domain.

126. The multivalent antigen-biding protein of claim 125, which consists essentially of a first polypeptide and a second polypeptide, wherein the first polypeptide consists essentially of (from N-terminal to C-terminal) a first heavy chain immunoglobulin single variable domain (V_{H}1sd) fused directly or through a linker to a first multimerizing component M1, which is fused directly or through a linker to a second heavy chain immunoglobulin single variable domain; and a second polypeptide consisting essentially of (from N-terminal to C-terminal) a third heavy chain immunoglobulin single variable domain fused directly or through a linker to a second multimerizing component M2.

127. The multivalent antigen-binding protein of claim 125, which consists essentially of the first polypeptide and the second polypeptide.

128. The multivalent antigen-binding protein of claim 125, wherein M1 and M2 each comprise a heavy chain domain selected from a hinge domain, a C_{H}2 domain, a C_{H}3 domain, and a combination thereof.

129. The multivalent antigen-binding protein of claim 125, wherein the first, second, and third, heavy chain immunoglobulin single variable domains are human.

130. The multivalent antigen-binding protein of claim 125, wherein the antigen-binding protein binds three different epitopes or two different epitopes.

131. The multivalent antigen-binding protein of claim 125, wherein the antigen-binding protein binds at least two different antigens.

132. A multivalent antigen-binding protein comprising a first polypeptide and a second polypeptide, wherein:
the first polypeptide comprises (from N-terminal to C-terminal) a first light chain immunoglobulin single variable domain (V_{L}sd1) fused directly or through a linker to a first multimerizing component M1, which is fused directly or through a linker to a second light chain immunoglobulin single variable domain (V_{L}sd2); and,
the second polypeptide comprises (from N-terminal to C-terminal) a third light chain immunoglobulin single variable domain (V_{L}sd3) fused directly or through a linker to a second multimerizing component M2.

133. The multivalent antigen-binding protein of claim 132, which consists essentially of a first polypeptide and a second polypeptide, wherein the first polypeptide consists essentially of a first light chain immunoglobulin single variable domain (V_{L}sd1) fused directly or through a linker to the first multimerizing component M1, which is fused directly or through a linker to a second light chain immunoglobulin single variable domain (V_{L}sd2); and the second polypeptide consists essentially of a third light chain immunoglobulin variable domain that is fused directly or through a linker to the second multimerizing component M2.

134. The multivalent antigen-binding protein of claim 132, which lacks an immunoglobulin heavy chain variable domain.

135. The multivalent antigen-binding domain of claim 132, wherein V_{L}sd1, V_{L}sd2, and V_{L}sd3 bind three separate antigens.

136. The multivalent antigen-binding protein of claim 132, wherein V_{L}sd1, V_{L}sd2, and V_{L}sd3 bind three epitopes on two antigens; optionally one antigen contains two epitopes and two of V_{L}sd1, V_{L}sd2, and V_{L}sd3 bind the two epitopes of the one antigen.

137. A multispecific antigen-binding protein comprising:
a first polypeptide comprising a first scFv, optionally a linker, a first multimerizing component (M1), optionally a linker, and a second scFv; and,
a second polypeptide that consists essentially of a third scFv, optionally a linker, and a second multimerizing component (M2).

138. The multispecific antigen-binding protein of claim 137, which consists essentially of a first polypeptide and a second polypeptide, wherein the first polypeptide consists essentially of a first scFv (scFv1) fused directly or through a linker to the first multimerizing component M1, which is fused directly or through a linker to a second scFv; (scFv2) and a second polypeptide consisting essentially of a third scFv (scFv3) fused directly or through a linker to the second multimerizing component M2.

139. The multispecific antigen-binding protein of claim 137, wherein scFv1, scFv2, and scFv3 each bind a different antigen; optionally two of scFv1, scFv2, and scFv3 bind the same first antigen optionally at different epitopes, and the remaining scFv binds a second, different antigen.

140. A multivalent antigen-binding protein, wherein the protein:
binds a first antigen (AG1) through a first immunoglobulin heavy chain variable domain (V_{H}), wherein AG1 does not bind a light chain variable domain of the antigen-binding protein; and,
binds a second antigen (AG2) through a first immunoglobulin light chain variable domain, wherein AG2 does not bind V_{H}.

141. The multivalent antigen-binding protein of claim 140, which consists essentially of four polypeptides, wherein:
the first polypeptide consists essentially of (from N-terminal to C-terminal) a human heavy chain single variable domain that binds AG1, fused directly or through a linker to a C_{H}1 sequence, which is fused directly or through a linker to a first multimerizing component M1;
the second polypeptide is identical to the first polypeptide, except that the C_{H}1 region is fused directly or through a linker to a second multimerizing component M2; and,
two copies of a third polypeptide, wherein the third polypeptide consists essentially of a human immunoglobulin Vκ single variable domain that binds AG2, wherein AG2 does not bind V_{H}, and wherein the Vκ single variable domain is fused directly or through a linker to a C_{L}.

142. The multivalent antigen-binding protein of claim 141, which binds AG1 employing the first polypeptide, and simultaneously binds AG2 employing the second polypeptide.

143. 4. The multivalent antigen-binding protein of claim 140, which consists essentially of the first polypeptide, the second polypeptide, and the third polypeptide, and the fourth polypeptide.

144. The multivalent antigen-binding protein of claim 140, wherein M1 and M2 each comprise a heavy chain domain selected from a hinge domain, a C_{H}2 domain, a C_{H}3 domain, and a combination thereof.

145. The multivalent antigen-binding protein of claim 140, wherein the first, second, third, and fourth polypeptides are human immunoglobulin sequences.

146. The multivalent antigen-binding protein of claim 140, wherein the light chain variable domain of the third polypeptide is encoded by a rearranged human nucleic acid sequence derived from a human light chain gene segment selected from a human Vκ1-39 gene segment and a human J segment, a human Vλ3-20 gene segment and a human J segment, a human Vλ1-40 gene segment and a human J segment, and a human Vλ2-14 gene segment and a human J segment.

147. The multivalent antigen-binding protein of claim 140, wherein the third polypeptide comprises a light chain constant domain selected from a human Cκ domain and a human Cλ domain.

148. A multivalent antigen-binding protein comprising four polypeptides, wherein:
a first polypeptide comprises an immunoglobulin heavy chain variable domain that binds a first antigen (AG1), a C_{H}1 domain, and a multimerizing component, wherein the first polypeptide multimerizes with a second polypeptide that is identical to the first polypeptide to form a dimer, wherein the dimerizing is mediated by a multimerizing component M1 on the first polypeptide and M2 on the second polypeptide;
a third and a fourth polypeptide, which are substantially identical, and which comprise a common light chain having a light chain variable domain fused to a light chain constant domain, wherein the light chain variable domain does not interfere with the binding of AG1 to thee heavy chain variable domain.

149. The multivalent antigen-binding protein of claim 148, wherein the heavy chain variable domain, the light chain variable domain, the C_{H}1, and the light chain constant domain are human.

150. The multivalent antigen-binding protein of claim 148, wherein the multimerizing component comprises a human immunoglobulin sequence selected from a hinge, a C_{H}2, a C_{H}3, and a combination thereof.

151. A multivalent antigen-binding protein comprising four polypeptides, wherein:
a first polypeptide comprises a κ light chain variable domain that specifically binds an antigen AG2, wherein the variable domain is fused to a C_{H}1 domain, which is fused directly or through a linker to a first multimerizing component M1;
a second polypeptide that is substantially identical to the first polypeptide, and comprises a multimerizing component M2;
a third polypeptide and a fourth polypeptide that are substantially identical, and comprise a common light chain comprising a light chain variable domain that does not interfere with the binding of AG2 to the κ light chain variable domain, wherein the light chain variable domain does not bind AG2.

152. The multivalent antigen-binding protein of claim 151, which consists essentially of the first polypeptide, the second polypeptide, the third polypeptide, and the fourth polypeptide.

153. The multivalent antigen-binding protein of claim 151, wherein M1 and M2 each comprise a heavy chain domain selected from a hinge domain, a C_{H}2 domain, a C_{H}3 domain, and a combination thereof.

154. The multivalent antigen-binding protein of claim 151, wherein the first, second, third, and fourth polypeptides are human immunoglobulin sequences.

155. The multivalent antigen-binding protein of claim 151, wherein the light chain variable domain of the third and fourth polypeptide is encoded by a rearranged human nucleic acid sequence derived from a human light chain gene segment selected from a human Vκ1-39 gene segment and a human J segment, a human Vκ3-20 gene segment and a human J segment, a human Vλ1-40 gene segment and a human J segment, and a human Vλ2-14 gene segment and a human J segment.

156. The multivalent antigen-binding protein of claim 151, wherein the third polypeptide comprises a light chain constant domain selected from a human Cκ domain and a human Cλ domain.

157. A multivalent antigen-binding protein according to any of the preceding claims that comprises a dimeric polypeptide that comprises a heavy chain immunoglobulin hinge region and a multimerizing component comprising a C_{H}2 and a C_{H}3 domain, wherein the hinge region is a chimeric, wherein the chimerism is characterized by a human IgG1 or a human IgG4 upper hinge amino acid sequence from positions 216 to 227 (EU numbering), the C_{H}2 domain comprises a human IgG4 C_{H}2 domain amino acid sequence from positions 237 to 340 (EU numbering), and the C_{H}3 domain comprises a human IgG1 or a human IgG4 C_{H}3 domain sequence from positions 341 to 447 (EU numbering).

158. The multivalent antigen-binding protein of claim 157, wherein the C_{H}1 domain comprises the amino acid sequence DKKV, and the chimeric hinge comprises the amino acid sequence EPKSCDKTHTCPPCPAPPVA.

159. The multivalent antigen-binding protein of claim 157, wherein the C_{H}1 domain comprises the amino acid sequence DKRV, and the chimeric hinge comprises the amino acid sequence ESKYGPPCPPCPAPPVA.

160. A multivalent antigen-binding protein according to any of the preceding claims, wherein the M1 and the M2 each comprise a C_{H}2 and a C_{H}3 domain, wherein either M1 or M2, but not both, are modified to eliminate or reduce binding to protein A.
